(19) United States
(12) Patent Application Publication
(10) Pub. No.: US 2016/0101072 A1
(43) Pub. Date: Apr. 14, 2016

(54) Therapeutic uses of enzyme inhibitors

(71) Applicant: Proximagen Limited, Cambridge (GB)

(72) Inventors: Kenneth Mulvany, London (GB); Martyn Pritchard, St. Ives (GB)

(21) Appl. No.: 14/757,224

(22) Filed: Dec. 7, 2015

Related U.S. Application Data
(63) Continuation-in-part of application No. 14/895,705, filed on Dec. 3, 2015.

(30) Foreign Application Priority Data
Jun. 12, 2013 (GB) 1310488.0
Dec. 12, 2013 (GB) 1322036.3
Dec. 8, 2014 (GB) 1421812.7

(51) Int. Cl. A61K 31/165; A61K 31/198; A61K 38/02; A61K 31/573

(52) U.S. Cl. CPC A61K 31/165; A61K 31/573; A61K 31/198; A61K 38/02

(57) ABSTRACT

The invention relates to use of compounds which inhibit VAP-1/SSAO activity for the treatment of muscular dystrophy. The invention also relates to combined preparations comprising compounds which inhibit VAP-1/SSAO activity, and their use for the treatment of muscular dystrophy.

### CROSS-REFERENCE TO RELATED APPLICATIONS

This application claims priority under 35 U.S.C. §119 to United Kingdom patent application GB1421812.7, filed on 8 Dec. 2014, and is a continuation-in-part of and claims the benefit of US patent application Ser. No. 14/895,705, filed on 3 Dec. 2015, which in turn is the National Stage of International Application No. PCT/GB2014/051817, filed on Jun. 12, 2014, which in turn claims priority to United Kingdom patent applications 1310488.0, filed 12 Jun. 2013, and 1322036.3, filed on 12 Dec. 2013. The entire disclosure of each of the fore mentioned applications is incorporated herein by reference.

### FIELD OF THE INVENTION

This invention relates to the use of a compound which inhibits VAP-1/SSAO activity for the treatment of muscular dystrophy. The invention also relates to the use of pharmaceutical compositions comprising these compounds for the treatment of muscular dystrophy. The invention also relates to combined preparations, and their use for the treatment of muscular dystrophy.

### BACKGROUND OF THE INVENTION

Semicarbazide-sensitive amine oxidase (SSAO), otherwise known as Vascular Adhesion Protein-1 (VAP-1) or Amine Oxidase, Copper Containing 3 (AOC3), belongs to the copper-containing amine oxidase family of enzymes (EC.1.4.3.6). Members of this enzyme family are sensitive to inhibition by semicarbazide and utilize cupric ion and protein-derived topa quinone (TPQ) cofactor in the oxidative deamination of primary amines to aldehydes, hydrogen peroxide, and ammonia according to the following reaction:

R—CH₂—NH₂+O₂→R—CHO+H₂O₂+NH₃

Known substrates for human SSAO include endogenous methylamine and aminoacetone as well as some xenobiotic amines such as benzylamine [Lyles, Int. J. Biochem. Cell Biol. 1996, 28, 259-274; Klinman, Biochim. Biophys. Acta 2003, 1647(1-2), 131-137; Mátyus et al., Curr. Med. Chem. 2004, 11(10), 1285-1298; O'Sullivan et al., Neurotoxicology 2004, 25(1-2), 303-315]. In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain [Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562].

SSAO activity has been found in a variety of tissues including vascular and non-vascular smooth muscle tissue, endothelium, and adipose tissue [Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216]. In addition, SSAO protein is found in blood plasma and this soluble form appears to have similar properties as the tissue-bound form [Yu et al., Biochem. Pharmacol. 1994, 47, 1055-1059; Kurkijärvi et al., J. Immunol. 1998, 161, 1549-1557]. It has recently been shown that circulating human and rodent SSAO originates from the tissue-bound form [Göktürk et al., Am. J. Pathol. 2003, 163(5), 1921-1928; Abella et al., Diabetologia 2004, 47(3), 429-438; Stolen et al., Circ. Res. 2004, 95(1), 50-57], whereas in other mammals the plasma/serum SSAO is also encoded by a separate gene called AOC4 [Schwelberger, J. Neural. Transm. 2007, 114(6), 757-762].

The precise physiological role of this abundant enzyme has yet to be fully determined, but it appears that SSAO and its reaction products may have several functions in cell signalling and regulation. For example, recent findings suggest that SSAO plays a role in both GLUT4-mediated glucose uptake [Enrique-Tarancon et al., J. Biol. Chem. 1998, 273, 8025-8032; Morin et al., J. Pharmacol. Exp. Ther. 2001, 297, 563-572] and adipocyte differentiation [Fontana et al., Biochem. J. 2001, 356, 769-777; Mercier et al., Biochem. J. 2001, 358, 335-342]. In addition, SSAO has been shown to be involved in inflammatory processes where it acts as an adhesion protein for leukocytes [Salmi & Jalkanen, Trends Immunol. 2001, 22, 211-216; Salmi & Jalkanen, in “Adhesion Molecules: Functions and Inhibition” K. Ley (Ed.), 2007, pp. 237-251], and might also play a role in connective tissue matrix development and maintenance [Langford et al., Cardiovasc. Toxicol. 2002, 2(2), 141-150; Göktürk et al., Am. J. Pathol. 2003, 163(5), 1921-1928]. Moreover, a link between SSAO and angiogenesis has recently been discovered [Noda et al., FASEB J. 2008, 22(8), 2928-2935].

Several studies in humans have demonstrated that SSAO activity in blood plasma is elevated in conditions such as congestive heart failure, diabetes mellitus, Alzheimer's disease, and inflammation [Lewinsohn, Braz. J. Med. Biol. Res. 1984, 17, 223-256; Boomsma et al.,Cardiovasc. Res. 1997, 33, 387-391; Ekblom, Pharmacol. Res. 1998, 37, 87-92; Kurkijärvi et al.,J. Immunol. 1998, 161, 1549-1557; Boomsma et al., Diabetologia 1999, 42, 233-237; Meszaros et al.,Eur. J. Drug Metab. Pharmacokinet. 1999, 24, 299-302; Yu et al., Biochim. Biophys. Acta 2003, 1647(1-2), 193-199; Mátyus et al., Curr. Med. Chem. 2004, 11(10), 1285-1298; O'Sullivan et al.,Neurotoxicology 2004, 25(1-2), 303-315; del Mar Hernandez et al., Neurosci. Lett. 2005, 384(1-2), 183-187]. The mechanisms underlying these alterations of enzyme activity are not clear. It has been suggested that reactive aldehydes and hydrogen peroxide produced by endogenous amine oxidases contribute to the progression of cardiovascular diseases, diabetic complications and Alzheimer's disease [Callingham et al., Prog. Brain Res. 1995, 106, 305-321; Ekblom, Pharmacol. Res. 1998, 37, 87-92; Yu et al., Biochim. Biophys. Acta 2003, 1647(1-2), 193-199; Jiang et al., Neuropathol Appl Neurobiol. 2008, 34(2), 194-204]. Furthermore, the enzymatic activity of SSAO is involved in the leukocyte extravasation process at sites of inflammation where SSAO has been shown to be strongly expressed on the vascular endothelium [Salmi et al., Immunity 2001, 14(3), 265-276; Salmi & Jalkanen, in “Adhesion Molecules: Functions and Inhibition” K. Ley (Ed.), 2007, pp. 237-251]. Accordingly, inhibition of SSAO has been suggested to have a therapeutic value in the prevention of diabetic complications and in inflammatory diseases [Ekblom, Pharmacol. Res. 1998, 37, 87-92; Salmi et al., Immunity 2001, 14(3), 265-276; Salter-Cid et al., J. Pharmacol. Exp. Ther. 2005, 315(2), 553-562].

SSAO knockout animals are phenotypically overtly normal but exhibit a marked decrease in the inflammatory responses evoked in response to various inflammatory stimuli [Stolen et al., Immunity 2005, 22(1), 105-115]. In addition, antagonism of its function in wild type animals in multiple animal models of human disease (e.g. carrageenan-induced paw inflammation, oxazolone-induced colitis, lipopolysaccharide-induced lung inflammation, collagen-induced arthritis, endotoxin-induced uveitis) by the use of antibodies and/or small molecules has been shown to be protective in decreasing the leukocyte infiltration, reducing the severity of the disease phenotype and reducing levels of inflammatory cytokines and chemokines [Kirton et al., Eur. J. Immunol. 2005, 35(11), 3119-3130; Salter-Cid et al., J. Pharmacol. Exp. Ther. 2005, 315(2), 553-562; McDonald et al., Annual Reports in Medicinal Chemistry 2007, 42, 229-243; Salmi & Jalkanen, in “Adhesion Molecules: Functions and Inhibition” K. Ley (Ed.), 2007, pp. 237-251; Noda et al., FASEB J. 2008 22(4), 1094-1103; Noda et al., FASEB J. 2008, 22(8), 2928-2935]. This anti-inflammatory protection seems to be afforded across a wide range of inflammatory models all with independent causative mechanisms, rather than being restricted to one particular disease or disease model. This would suggest that SSAO may be a key nodal point for the regulation of the inflammatory response, and it seems therefore likely that SSAO inhibitors may be effective anti-inflammatory drugs in a wide range of human diseases.

Fibrosis can result from chronic tissue inflammation when the resolution of the inflammation is partly abrogated by the chronic nature of the inflammatory stimulus. The result can be inappropriate repair of the tissue with excessive extracellular matrix deposition (including collagen) with tissue scarring. This is a consequence of myofibroblast activation by stimuli including fibronectin and reactive oxygen species as well as growth factors such as transforming growth factor-β-1 (TGFβ-1), insulin-like growth factor-I (IGF-I), platelet-derived growth factor (PDGF) and connective tissue growth factor (CTGF) resulting in increased production of collagen, elastin, hyaluronan, glycoproteins and proteoglycans. In addition the activity of invading macrophages plays a crucial part in regulating the repair and fibrotic processes.

VAP-1 has also been implicated in the progression and maintenance of fibrotic diseases especially in the liver. Weston and Adams (J Neural Transm. 2011, 118(7), 1055-64) have summarised the experimental data implicating VAP-1 in liver fibrosis. Weston et al (EASL Poster 2010) showed highly increased expression of VAP-1 in human fibrotic liver, particularly associated with the activated myofibroblasts and collagen fibrils. This anatomical association with fibrosis was consistent with the observation that blockade of VAP-1 accelerated the resolution of carbon tetrachloride induced fibrosis, and suggested a role for the VAP-1/SSAO enzyme product H2O2 in the activation of the myofibroblasts.

The same authors also showed that the pro-fibrotic growth factor TGFβ increased the expression of VAP-1 in liver cells by approximately 50-fold.

There are a large number of diseases which cause wasting or atrophy of muscles and some of these are associated with significant amounts of fibrosis. The most well-known include Duchenne Muscular Dystrophy and Becker Muscular Dystrophy. These are both caused by defects in the muscle cytoskeletal protein dystrophin, and in the former usually results in death by the age of 25, while in the less severe Becker form, patients usually survive into old age. The pathological basis of both these diseases is considered to be a consequence of poor muscle cell connectivity to the extracellular matrix, resulting in the weakening of the sarcolemma and cell death. The muscle tissue then suffers from repeated cycles of cell death and aberrant repair, resulting in fibrosis and the replacement of muscle tissue by fat tissue (Mann et al., 2011, Skeletal Muscle. 1(1):21; Klinger et al. 2012 Acta Myol. 31(3): 184-189). The symptoms of these diseases include pain and muscle weakness. Other dystrophies arising from similar causes include limb girdle muscular dystrophy, congenital muscular dystrophy and distal muscular dystrophy. All of these appear to have defects in cell attachment to the extracellular matrix. Fibrosis is therefore a major issue in the muscular dystrophies and a therapeutic capable of reducing or reversing the fibrosis would be extremely beneficial to patients suffering from muscular dystrophy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows: (a) VAP-1 expression in a muscle tissue section of a boy with Duchenne Muscular Dystrophy (DMD); and (b) VAP-1 expression in a muscle tissue section of an age-matched boy with normal muscles;

FIG. 2 shows, at ten times and twenty times magnification, hematoxylin and eosin (H & E) staining of sections of diaphragms of mdx mice treated with: (a) vehicle; or (b) benserazide;

FIG. 3 shows, at twenty times magnification, staining of murine F4/80 antigen in sections of diaphragms of mdx mice treated with: (a) vehicle; or (b) benserazide;

FIG. 4 shows the effect of prednisolone, benserazide, and carbidopa, on recruitment of CD11b⁺ myeloid cells into muscles of mdx mice (*P<0.05, ** P<0.005, compared to vehicle);

FIG. 5 shows the effect of prednisolone, benserazide, and carbidopa, on recruitment of: (A) CD11b⁺ Ly6C^{high} profibrotic myeloid cells; and (B) CD11 b⁺ Ly6C^{low} anti-fibrotic myeloid cells; into muscles of mdx mice (*P<0.05, ** P<0.005 compared to vehicle);

FIG. 6 shows the effect of benserazide, carbidopa, and a combination of carbidopa and prednisolone, on recruitment of: (A) lymphoid CD4⁺CD8⁻ T cells; and (B) lymphoid CD4⁻CD8⁻ T cells; into muscles of mdx mice (*P<0.05, ** P<0.005 compared to vehicle);

FIG. 7 shows the effect of prednisolone, benserazide, carbidopa, and a combination of carbidopa and prednisolone, on mRNA levels of the following inflammatory markers in muscles of mdx mice: (A) TNF; (B) Lgals3; (C) CD53; (D) CD48; (E) CD11b (*P<0.05, ** P<0.005 compared to vehicle); and FIG. 8 shows the effect of prednisolone, benserazide, carbidopa, and a combination of carbidopa and prednisolone, on mRNA levels of the pro-fibrotic growth factor TGFβ in muscles of mdx mice (*P<0.05 compared to vehicle).

### DETAILED DESCRIPTION OF THE INVENTION

The invention described herein relates to the expression of VAP-1 in dystrophic muscle, which VAP-1 expression is expected to increase during the fibrotic disease process. In normal muscle the expression of VAP-1 is low, and largely restricted to the blood vessels (Salmi et al., 1993, J. Exp. Med. 178, 2255-2260) but increases in inflamed and fibrotic tissues. This increase in expression in the diseased state makes VAP-1 a promising therapeutic target in dystrophic muscle. Inhibition of VAP-1/SSAO is expected to reduce the concentration of pro-inflammatory and pro-fibrotic enzyme products (such as aldehydes, hydrogen peroxide and ammonia) whilst also decreasing the adhesive capacity of immune and myofibroblast cells and correspondingly their activation and invasion of the muscle tissue. Thus inhibition of VAP-1/SSAO is expected to be therapeutically beneficial in the treatment of muscle fibrosis and therefore muscular dystrophy.

In addition to treating the fibrosis component of muscular dystrophy, inhibition of VAP-1/SSAO is expected to have further beneficial effects on muscle tissue maintenance. VAP-1/SSAO inhibitors are known to reduce leukocyte and monocyte incursion into tissues. It is known from the mdx mouse model, a murine model of Duchenne Muscular Dystrophy, that partial inhibition of macrophage incursion into the muscle tissue has a beneficial effect on muscle tissue maintenance. Therefore VAP-1/SSAO inhibitors are expected to have therapeutic effects in dystrophic muscle by reducing leukocyte, and particularly monocyte, incursion into the tissue.

In summary, it is expected that VAP-1/SSAO inhibitors will reduce inflammation and muscle loss through inhibition of leukocyte invasion, and reduce muscular fibrosis and scarring through reduced VAP-1 activity in the muscle tissue, and reduce inflammatory and fibrotic cell activation in muscle tissue through reduced production of pro-inflammatory and pro-fibrotic enzyme products such as aldehydes, hydrogen peroxide and ammonia.

According to the present invention, there is provided a VAP-1 inhibitor for use in the treatment of muscular dystrophy.

In another aspect, the invention provides the use of a VAP-1 inhibitor in the manufacture of a medicament for treatment of muscular dystrophy.

In another aspect, the invention provides a method of treating muscular dystrophy comprising administering to a subject suffering such disease an effective amount of a VAP-1 inhibitor.

The Applicant has also found that, surprisingly, certain beneficial effects of VAP-1 inhibitors (especially the VAP-1 inhibitor carbidopa) are enhanced in the presence of a steroid, for example a glucocorticoid, such as prednisolone.

Accordingly, there is also provided according to the invention a combined preparation, which comprises: (a) a VAP-1 inhibitor compound; and (b) a steroid.

A combined preparation of the invention may be provided as a pharmaceutical combination for administration to a mammal, preferably a human.

According to the invention there is also provided a pharmaceutical composition, which comprises: (a) a VAP-1 inhibitor compound; and (b) a steroid; and (c) a pharmaceutically acceptable carrier, excipient, or diluent.

The VAP-1 inhibitor may optionally be provided together with a pharmaceutically acceptable carrier, excipient, or diluent, and/or the steroid may be provided together with a pharmaceutically acceptable carrier, excipient, or diluent.

According to the invention there is further provided a combined preparation of the invention, or a pharmaceutical composition of the invention, for use as a medicament.

In another aspect, the invention provides a combined preparation of the invention, or a pharmaceutical composition of the invention, for use in the treatment of muscular dystrophy.

In another aspect, the invention provides use of a combined preparation of the invention, or a pharmaceutical composition of the invention, in the manufacture of a medicament for the treatment of muscular dystrophy.

In another aspect, the invention provides a method of treating muscular dystrophy comprising administering to a subject suffering such disease an effective amount of a VAP-1 inhibitor compound and a steroid.

The steroid may be a glucocorticoid, such as prednisolone, or a pharmaceutically acceptable salt thereof, or prednisone, or a pharmaceutically acceptable salt thereof.

As used herein, the terms “treatment,” “treating,” “treat” and the like, refer to obtaining a desired pharmacologic and/or physiologic effect. The effect can be prophylactic in terms of completely or partially preventing muscular dystrophy or a symptom thereof and/or can be therapeutic in terms of a partial or complete cure for muscular dystrophy and/or an adverse effect attributable to the disease. “Treatment,” as used herein, covers any treatment of muscular dystrophy in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

An “effective amount” of a VAP-1 inhibitor refers to the amount of a VAP-1 inhibitor that, when administered to a mammal or other subject for treating muscular dystrophy, is sufficient to effect such treatment for the disease. The “effective amount” will vary depending on the VAP-1 inhibitor, the disease and its severity and the age, weight, etc., of the subject to be treated.

The term “VAP-1 inhibitor” or “VAP-1 inhibitor compound” includes both non-biological small molecule inhibitors of VAP-1 and biological inhibitors of VAP-1, including but not limited to RNA, antibodies, polypeptidic or proteinaceous inhibitors of VAP-1.

For present purposes, a “VAP-1 inhibitor” or “VAP-1 inhibitor compound” is one which has an IC50 value of less than 1000nM in the VAP-1 Assay described below.

VAP-1 Inhibitors

Small molecules of different structural classes have previously been disclosed as VAP-1 inhibitors, for example in WO 02/38153 (tetrahydroimidazo[4,5-c]pyridine derivatives), in WO 03/006003 (2-indanylhydrazine derivatives), in WO 2005/014530 (allylhydrazine and hydroxylamine (aminooxy) compounds) and in WO 2007/120528 (allylamino compounds), WO2011034078 (N-[3-(heterocyclyl or phenyl)benzyl]-2-aminoglycinamides), and WO2012120195 (Pyridazinones), and WO2012124696 (Guanidines), and Bioorganic & Medicinal Chemistry (2013), 21(13), 3873-3881 (1H-imidazol-2-amine derivatives), and Bioorganic & Medicinal Chemistry (2013), 21(5), 1219-1233 (Thiazoles).

Many further small molecule VAP-1 inhibitors are known, for example, haloallyl amines of WO2009066152; imidazopyridines of WO2010064020; dihydralazine (WO2010015870); pyrazolo[4,3-c]pyridines of WO2010031791; 4,5,6,7-tetrahydroimidazo[4,5-c]pyridines of US2002198189, WO0238153 and WO2010031789; oximes of WO2010029379; allyl hydrazine, hydroxylamine and other compounds of US2005096360, WO2006094201 and WO2005014530; amine, amide and allylamino compounds of WO2007120528, US2007078157, WO2005082343 and WO2009055002; hydroxamic acids of WO2006013209; vitamin B1, vitamin B1 derivatives and vitamin B1 precursors of WO2008025870; 2,3,4,6,8-pentamethoxyl-dibenzofuran (CN100486971); compounds of US2007066646; aminoglycosides of WO2005063261; carbocyclic hydrazino compounds of WO03006003; hydrazono compounds of US2004106654 and WO0202090; haloallylamines such as MDL72161A, MDL72274A and MDL72964A (mofegiline, (E)-4-fluoro-beta-fluoromethylene benzene butanamine hydrochloride, (E)-2-(4-fluorophenethyl)-3-fluoroallylamine hydrochloride) as in WO9323023, Lyles et al, Biochem. Pharmacol., 1987, 2847 and McDonald et al, J. Med. Chem., 1985, 186; thiazoles of WO2004087138, WO2004067521, WO2005089755, WO2006011631 and WO2006028269; semicarbazide and hydrazines (e.g. phenylhydrazine, phenelzine, carbazine and hydrazaline) as in McDonald et al, Annual reports in medicinal chemistry, 42, 229-243, 2007; hydrazines of WO2004104191 and WO2002002541; 1,3,4-oxadiazine compounds of WO200202541; hydrazino alcohol derivatives of WO2005080319; propargylamines of Sayre et al, Biochem., Biophys., Res. Commun, 2003, 788, Sayre et al, Bioorg. Med. Chem., 2006, 1444 and Sayre et al, Eur. J. Biochem., 2002, 3645; peptides as in Yegutkin, Eur. J. Immunol., 2004, 2276; dihydropyrroles of US20060025438 and Sayre et al, J. Am. Chem. Soc., 2002, 12135; proline amide derivatives of Sayre et al, Bioorg. Med. Chem., 2007, 1868; benzene and thiophene derivatives of WO2009145360 and WO 2009096609; thiazoles of US20040259923; and also includes molecules such as 5-hydroxytryptamine, 3-bromopropylamine, N-(phenyl-allyl)-hydrazine HCl (LJP-1207), 2-hydrazinopyridine, MDL-72274 ((E)-2-phenyl-3-chloroallylamine hydrochloride), MDL-72214 (2-phenylallylamine), MDL-72145, MDL-72161, mexiletine, isoniazid, imipramine, maprotiline, zimeldine, nomifensine, azoprocarbazine, monomethylhydrazine, dl-alphamethyltryptamine, dl-alphamethylbenzylamine, MD780236 (Dostert et al, J. Pharmacy & Pharmacol., 1984, 782), Z-3-Fluoro-2-(4-methoxybenzyl)allylamine hydrochloride (LJP-1586) (O'Rourke et al, JPET, 2008, 867), 2-(dimethyl(2-phenylethyl)silyl)methanamine, cuprozine, alkylamino derivatives of 4-aminomethylpyridine (Bertini et al, J. Med. Chem., 2005, 664), (1S,2S)-2-(1-methylhydrazino)-1-indanol (BTT-2052) (Marttila-Ichihara et al, JI, 2010, 2741), RTU-1096, kynuramine and carbidopa.

Biological inhibitors of VAP-1 include but are not limited to antibodies to VAP-1, RNAi, siRNA (examples of siRNAs suitable for targeting VAP-1 are described, for example, in WO2006134203), anti-sense oligonucleotides, anti-sense peptidyl nucleic acids, and aptamers. Examples of VAP-1 antibodies include but are not limited to anti-VAP-1 neutralizing antibody (available, for example, from R&D systems, Minneapolis, MN, catalogue numbers. AF3957, MAB39571 and MAB3957; Everest Biotech , Oxford, UK, catalogue number EB07582; and antibodies identified in WO2008129124, WO2003093319 and Koskinen et al, Blood, 2004, 3388, Arvilommi et al, Eur. J. Immunol., 1996, 825, Salmi et al, J. Exp. Med., 1993, 2255 and Kirten et al, Eur. J. Immunol., 2005, 3119.

The VAP-1 inhibitors disclosed specifically or generically in the above publications are expected to have utility in the treatment of muscular dystrophy according to the present invention.

Specific Examples of VAP-1 inhibitor compounds suitable for use in the present invention are provided below. Any pharmaceutically acceptable salt form of the Examples is suitable for use in the present invention. Specific examples of inhibitors of VAP-1 include the compounds speficially disclosed as Examples in WO 2010/031789, namely:

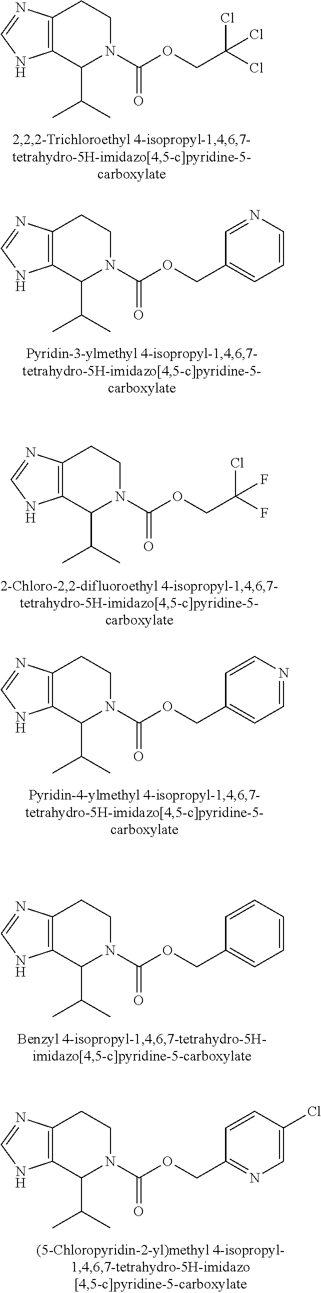 [SMILES: CC(C)C1c2[nH]cnc2CCN1C(=O)OCC(Cl)(Cl)Cl ; CC(C)C1c2[nH]cnc2CCN1C(=O)OCc1cccnc1 ; CC(C)C1c2[nH]cnc2CCN1C(=O)OCC(F)(F)Cl ; CC(C)C1c2[nH]cnc2CCN1C(=O)OCc1ccncc1 ; CC(C)C1c2[nH]cnc2CCN1C(=O)OCc1ccccc1 ; CC(C)C1c2[nH]cnc2CCN1C(=O)OCc1ccc(Cl)cn1]

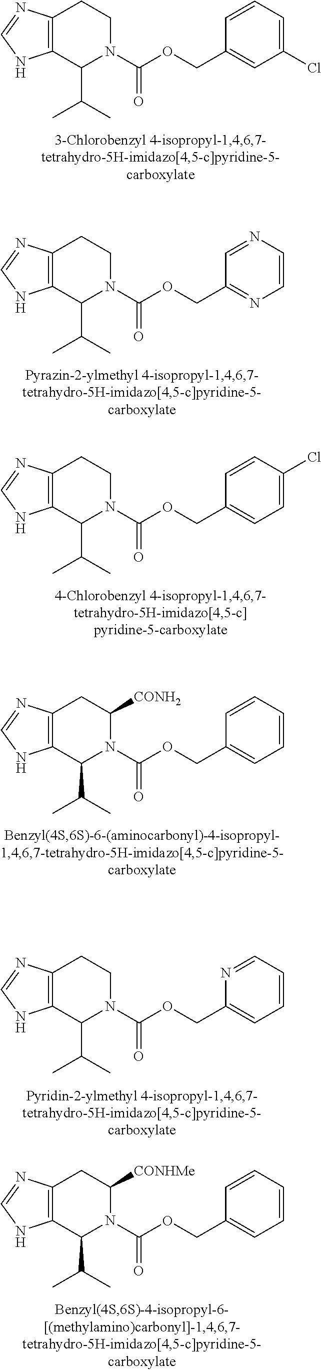

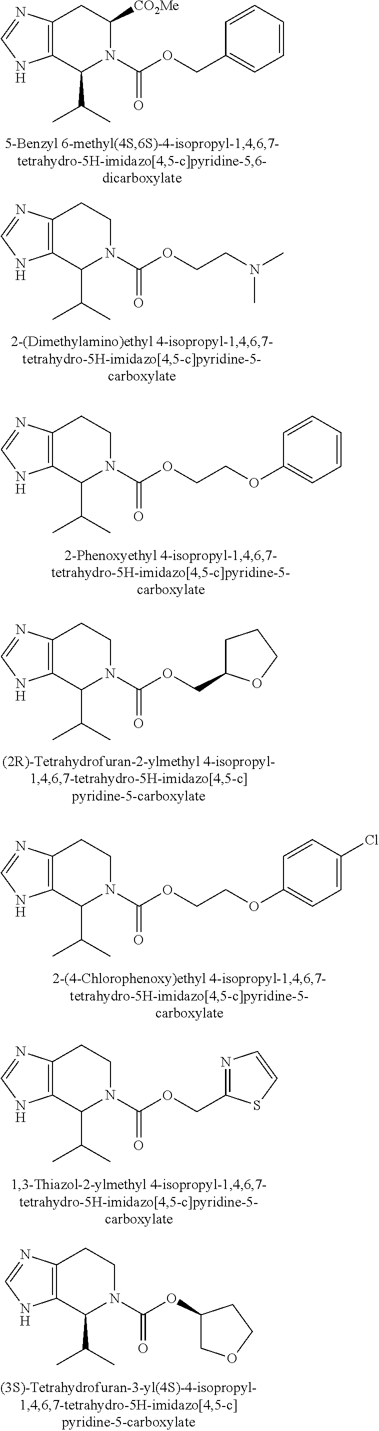

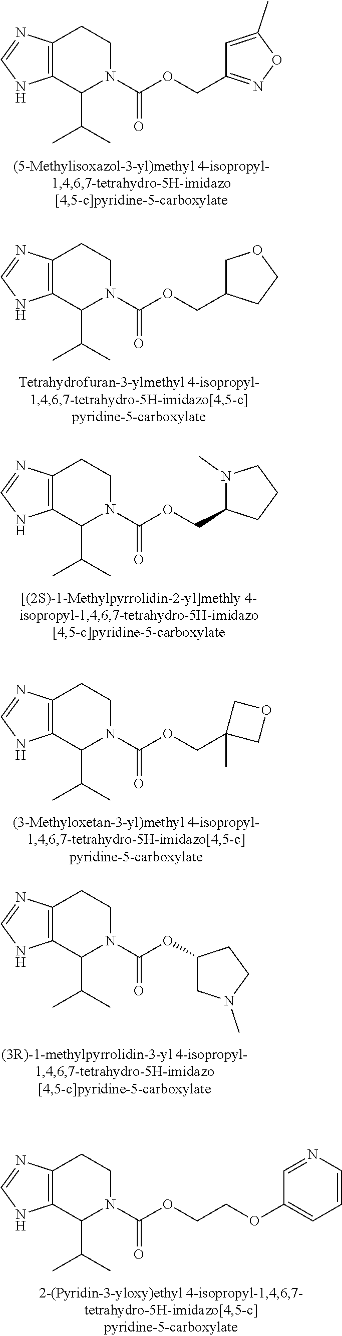 [SMILES: Cc1cc(COC(=O)N2CCc3nc[nH]c3C2C(C)C)no1 ; CC(C)C1c2[nH]cnc2CCN1C(=O)OCC1CCOC1 ; CC(C)C1c2[nH]cnc2CCN1C(=O)OC[C@@H]1CCCN1C ; CC(C)C1c2[nH]cnc2CCN1C(=O)OCC1(C)COC1 ; CC(C)C1c2[nH]cnc2CCN1C(=O)O[C@@H]1CCN(C)C1 ; CC(C)C1c2[nH]cnc2CCN1C(=O)OCCOc1cccnc1]

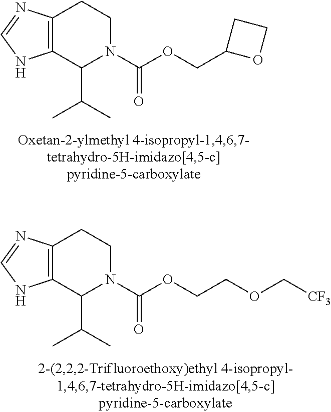 [SMILES: CC(C)C1c2[nH]cnc2CCN1C(=O)OCC1CCO1 ; CC(C)C1c2[nH]cnc2CCN1C(=O)OCCOCC(F)(F)F]

Other specific examples of inhibitors of VAP-1 include the following, which are Examples from WO2011/113798:

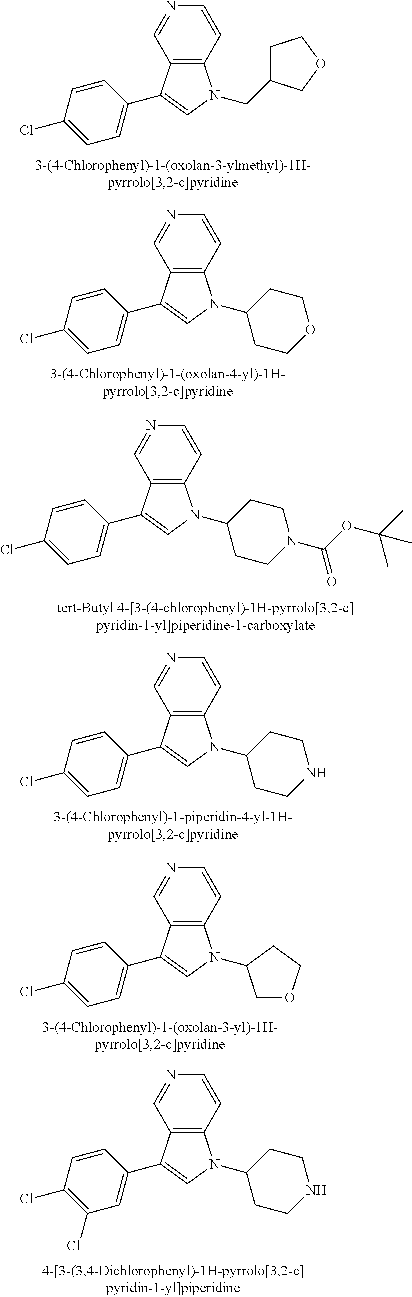 [SMILES: Clc1ccc(-c2cn(CC3CCOC3)c3ccncc23)cc1 ; Clc1ccc(-c2cn(C3CCOCC3)c3ccncc23)cc1 ; CC(C)(C)OC(=O)N1CCC(n2cc(-c3ccc(Cl)cc3)c3cnccc32)CC1 ; Clc1ccc(-c2cn(C3CCNCC3)c3ccncc23)cc1 ; Clc1ccc(-c2cn(C3CCOC3)c3ccncc23)cc1 ; Clc1ccc(-c2cn(C3CCNCC3)c3ccncc23)cc1Cl]

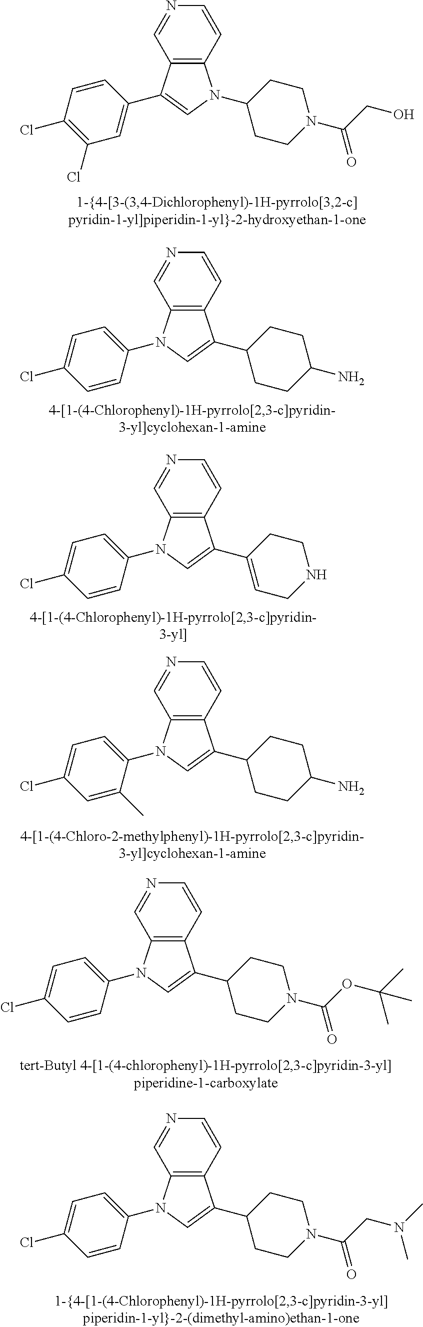 [SMILES: O=C(CO)N1CCC(n2cc(-c3ccc(Cl)c(Cl)c3)c3cnccc32)CC1 ; NC1CCC(c2cn(-c3ccc(Cl)cc3)c3cnccc23)CC1 ; Clc1ccc(-n2cc(C3=CCNCC3)c3ccncc32)cc1 ; Cc1cc(Cl)ccc1-n1cc(C2CCC(N)CC2)c2ccncc21 ; CC(C)(C)OC(=O)N1CCC(c2cn(-c3ccc(Cl)cc3)c3cnccc23)CC1 ; CN(C)CC(=O)N1CCC(c2cn(-c3ccc(Cl)cc3)c3cnccc23)CC1]

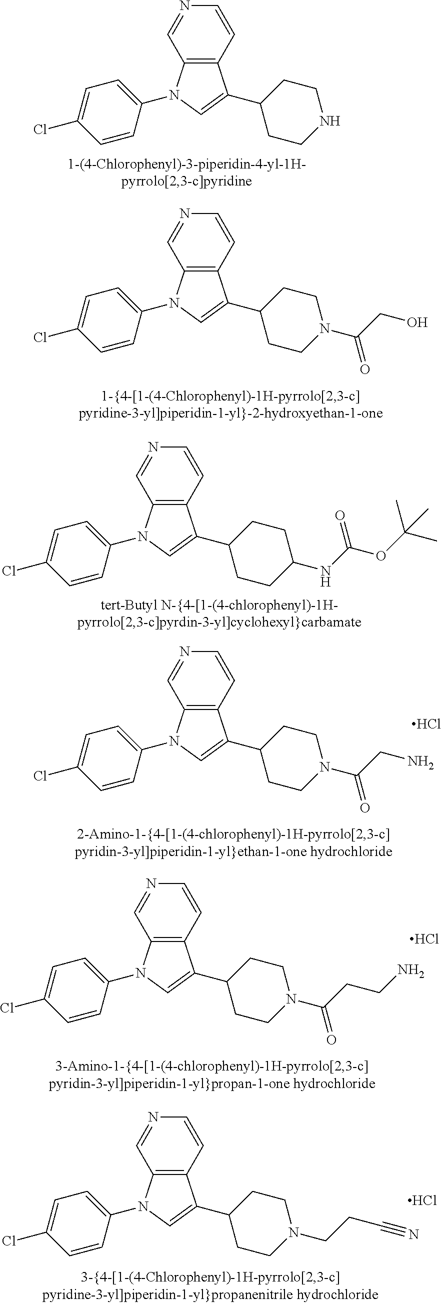

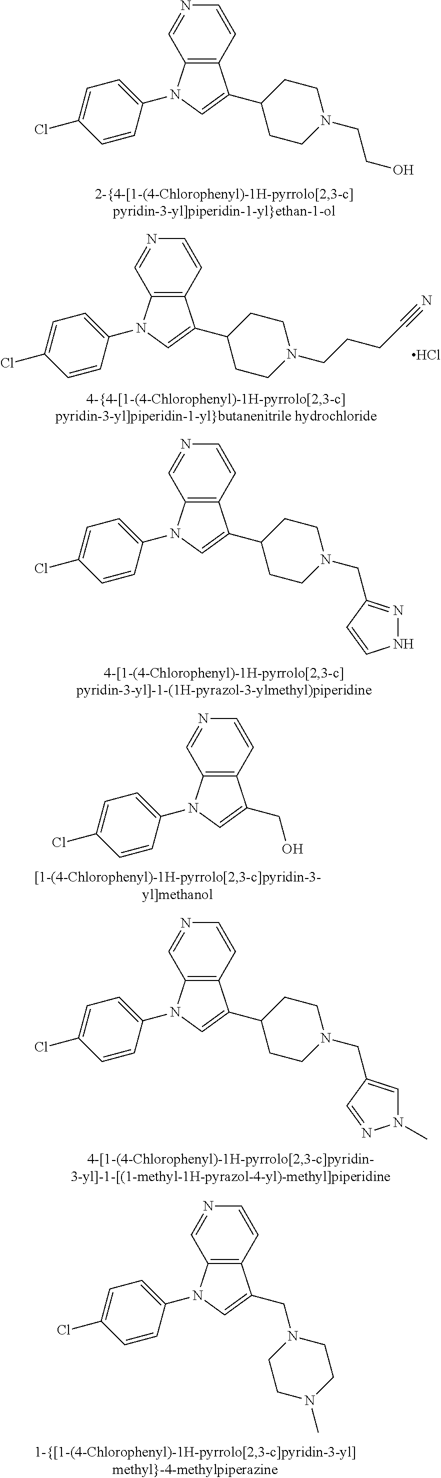

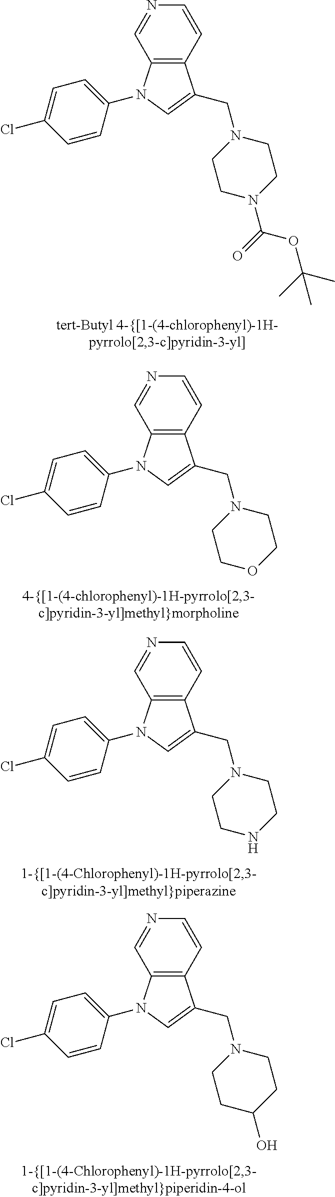 [SMILES: CC(C)(C)OC(=O)N1CCN(Cc2cn(-c3ccc(Cl)cc3)c3cnccc23)CC1 ; Clc1ccc(-n2cc(CN3CCOCC3)c3ccncc32)cc1 ; Clc1ccc(-n2cc(CN3CCNCC3)c3ccncc32)cc1 ; OC1CCN(Cc2cn(-c3ccc(Cl)cc3)c3cnccc23)CC1]

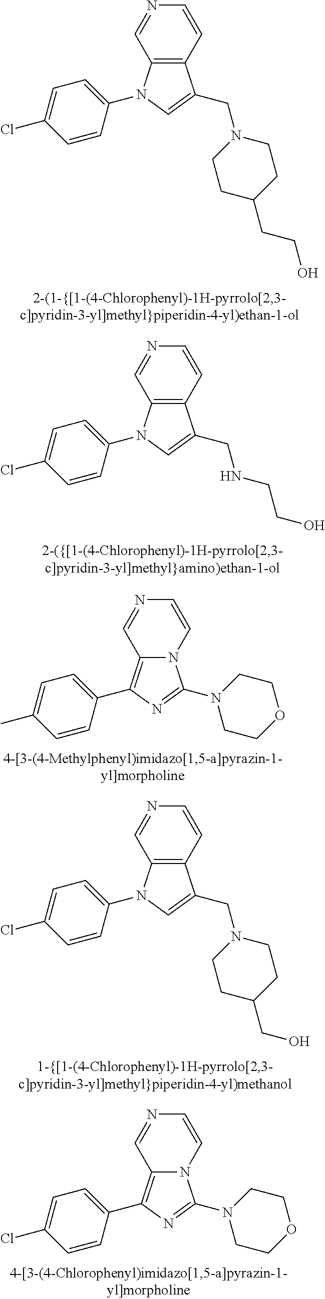 [SMILES: OCCC1CCN(Cc2cn(-c3ccc(Cl)cc3)c3cnccc23)CC1 ; OCCNCc1cn(-c2ccc(Cl)cc2)c2cnccc12 ; Cc1ccc(-c2nc(N3CCOCC3)n3ccncc23)cc1 ; OCC1CCN(Cc2cn(-c3ccc(Cl)cc3)c3cnccc23)CC1 ; Clc1ccc(-c2nc(N3CCOCC3)n3ccncc23)cc1]

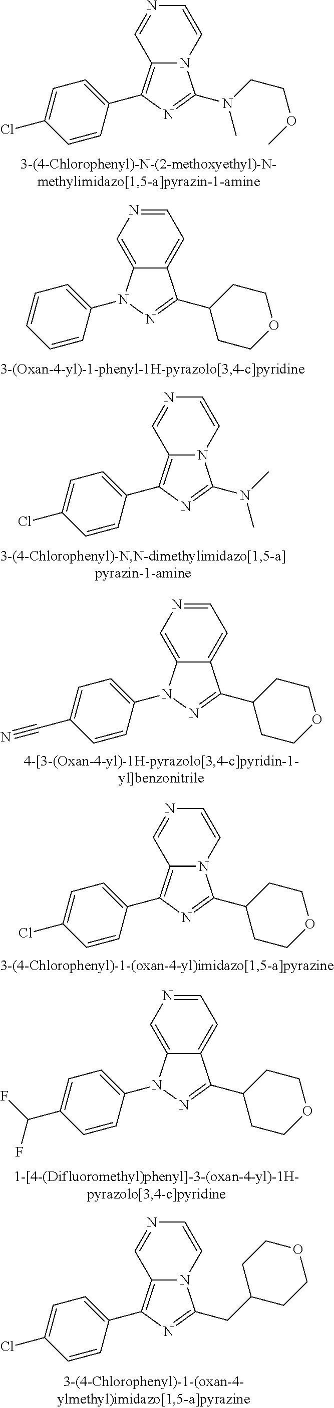

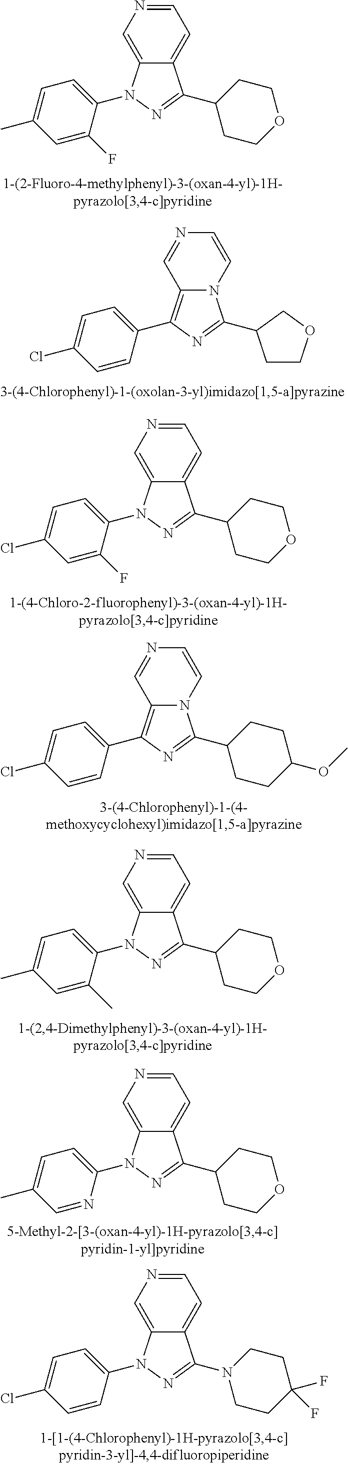

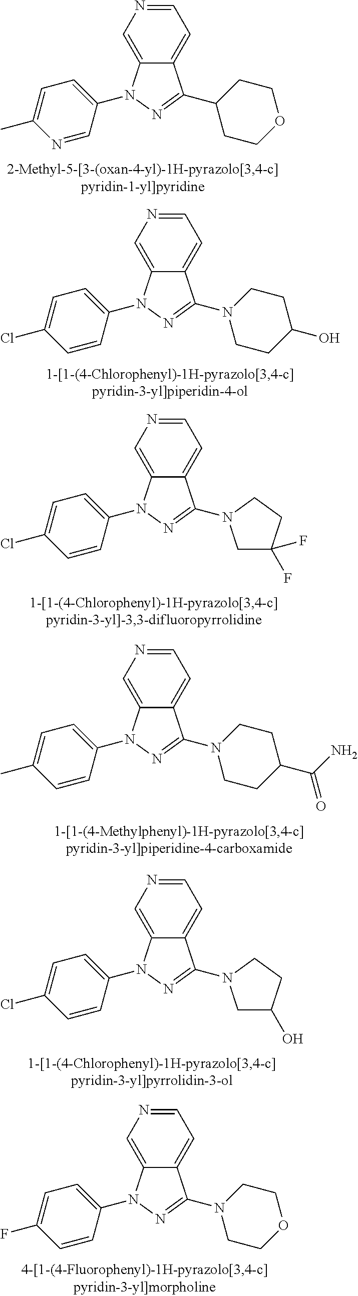 [SMILES: Cc1ccc(-n2nc(C3CCOCC3)c3ccncc32)cn1 ; OC1CCN(c2nn(-c3ccc(Cl)cc3)c3cnccc23)CC1 ; FC1(F)CCN(c2nn(-c3ccc(Cl)cc3)c3cnccc23)C1 ; Cc1ccc(-n2nc(N3CCC(C(N)=O)CC3)c3ccncc32)cc1 ; OC1CCN(c2nn(-c3ccc(Cl)cc3)c3cnccc23)C1 ; Fc1ccc(-n2nc(N3CCOCC3)c3ccncc32)cc1]

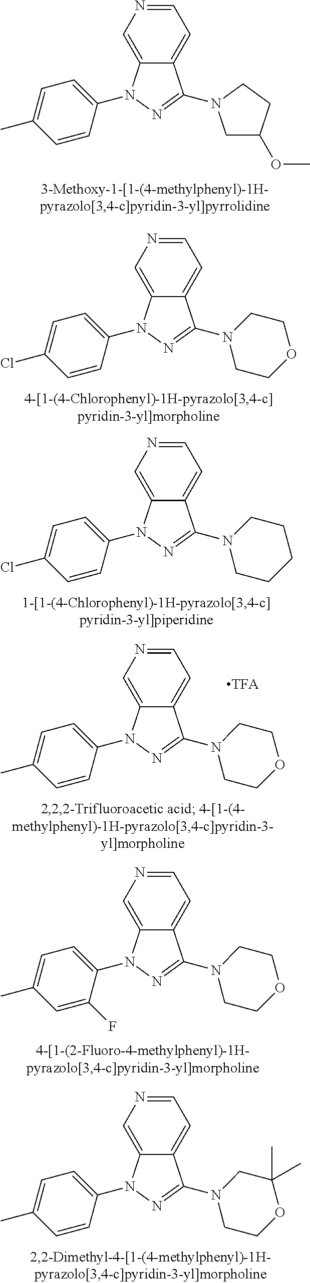

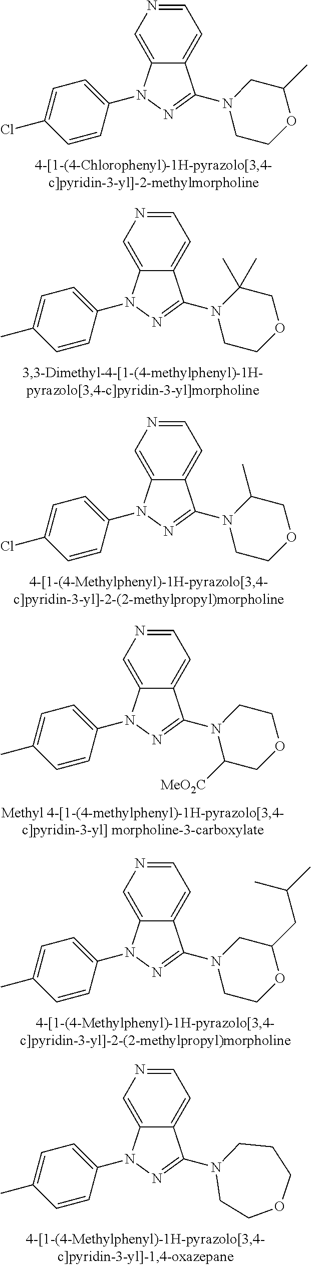

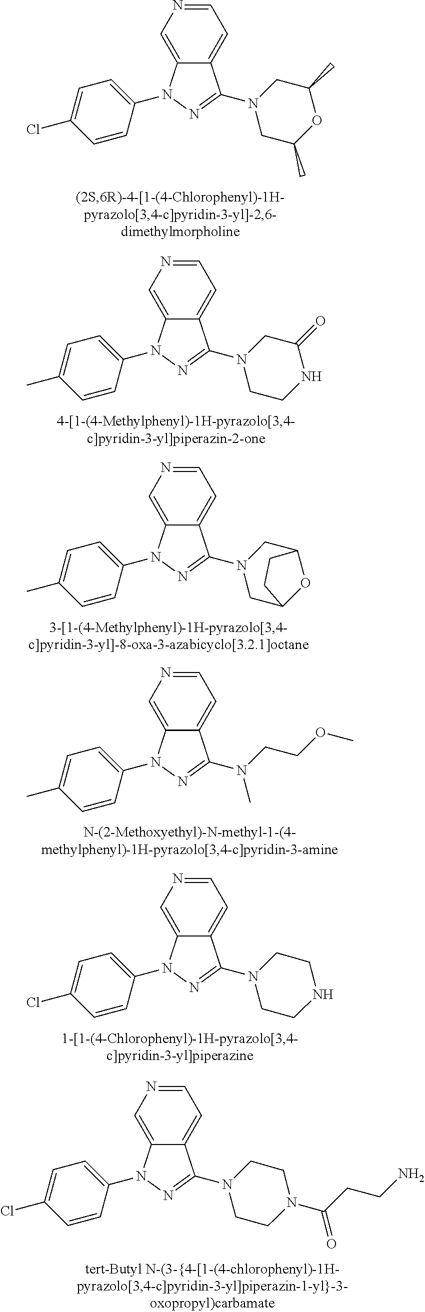

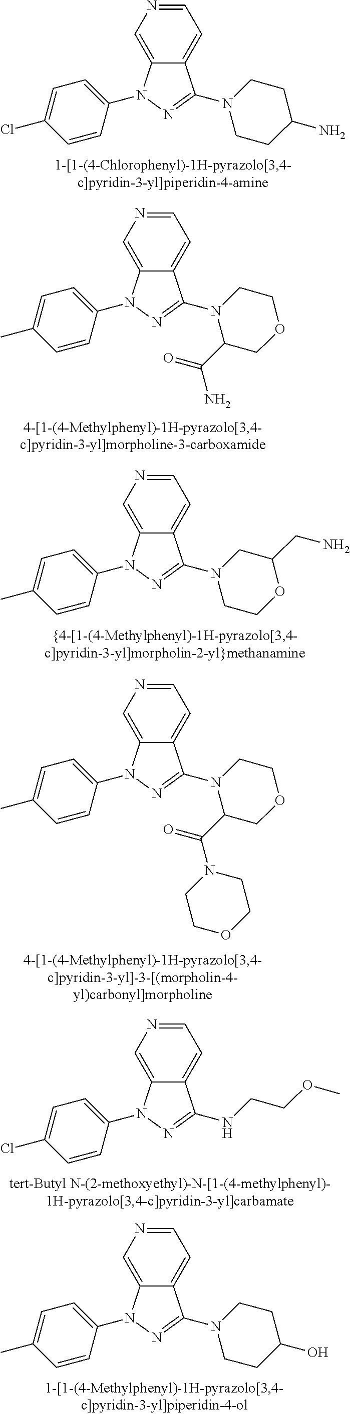

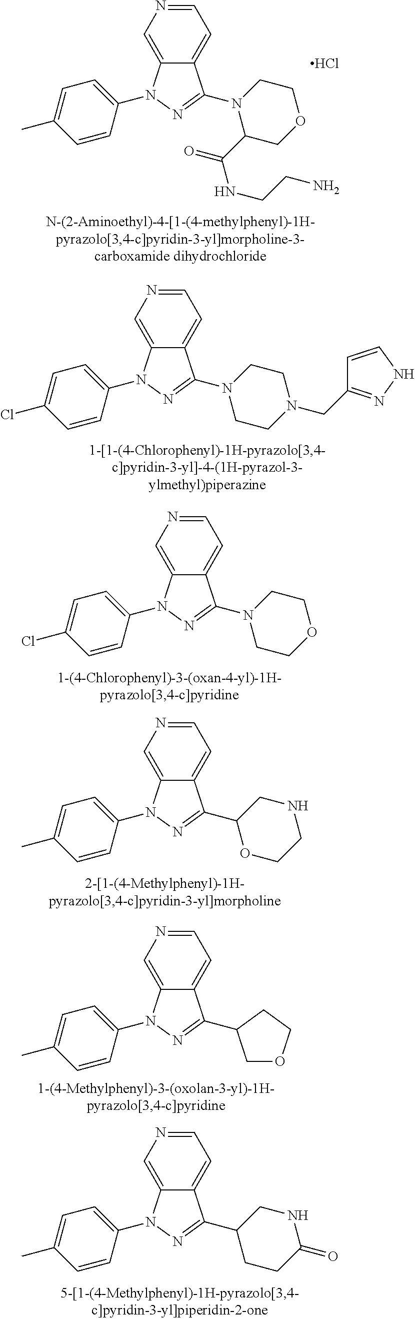

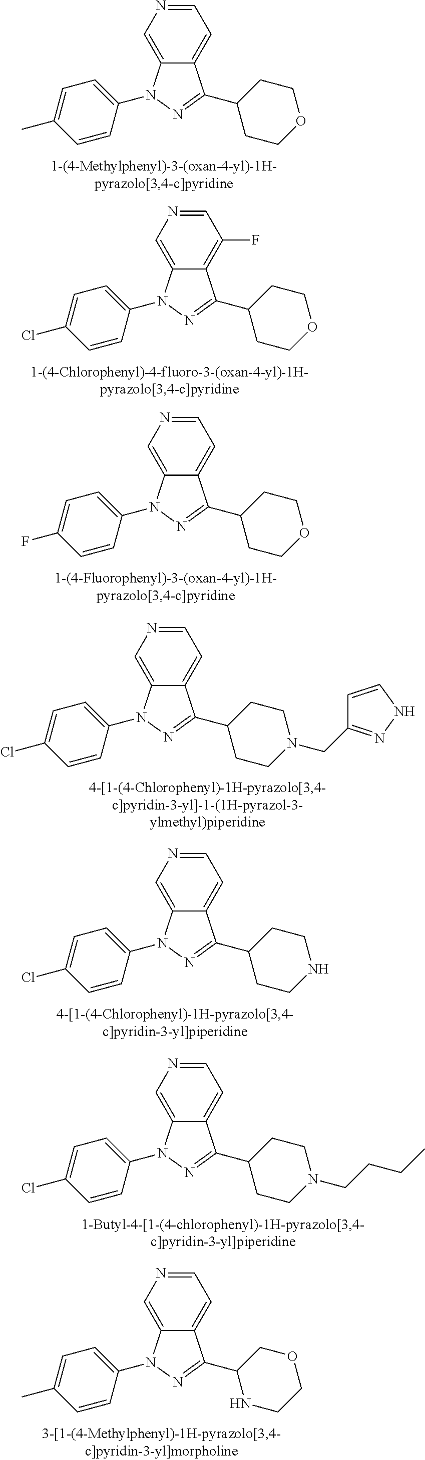

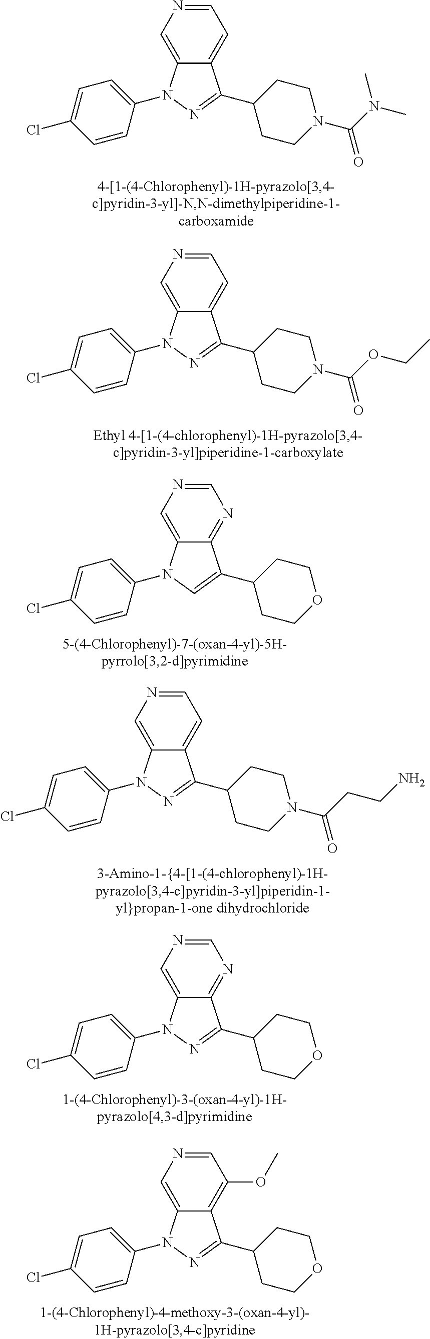 [SMILES: CN(C)C(=O)N1CCC(c2nn(-c3ccc(Cl)cc3)c3cnccc23)CC1 ; CCOC(=O)N1CCC(c2nn(-c3ccc(Cl)cc3)c3cnccc23)CC1 ; Clc1ccc(-n2cc(C3CCOCC3)c3ncncc32)cc1 ; NCCC(=O)N1CCC(c2nn(-c3ccc(Cl)cc3)c3cnccc23)CC1 ; Clc1ccc(-n2nc(C3CCOCC3)c3ncncc32)cc1 ; COc1cncc2c1c(C1CCOCC1)nn2-c1ccc(Cl)cc1]

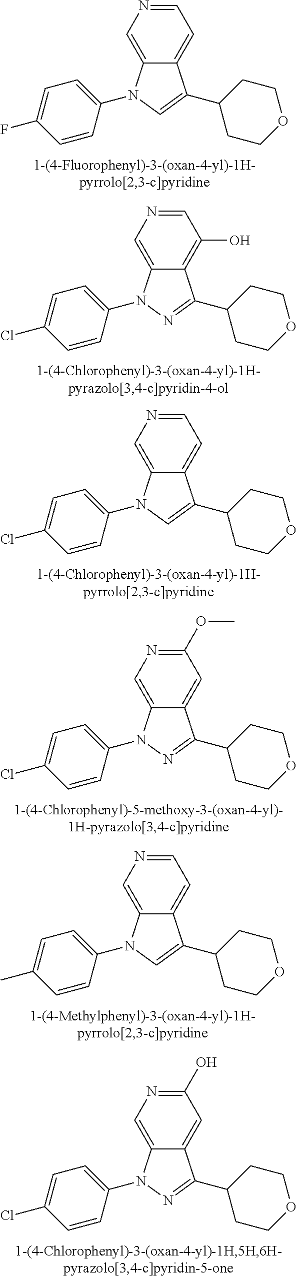 [SMILES: Fc1ccc(-n2cc(C3CCOCC3)c3ccncc32)cc1 ; Oc1cncc2c1c(C1CCOCC1)nn2-c1ccc(Cl)cc1 ; Clc1ccc(-n2cc(C3CCOCC3)c3ccncc32)cc1 ; COc1cc2c(C3CCOCC3)nn(-c3ccc(Cl)cc3)c2cn1 ; Cc1ccc(-n2cc(C3CCOCC3)c3ccncc32)cc1 ; Oc1cc2c(C3CCOCC3)nn(-c3ccc(Cl)cc3)c2cn1]

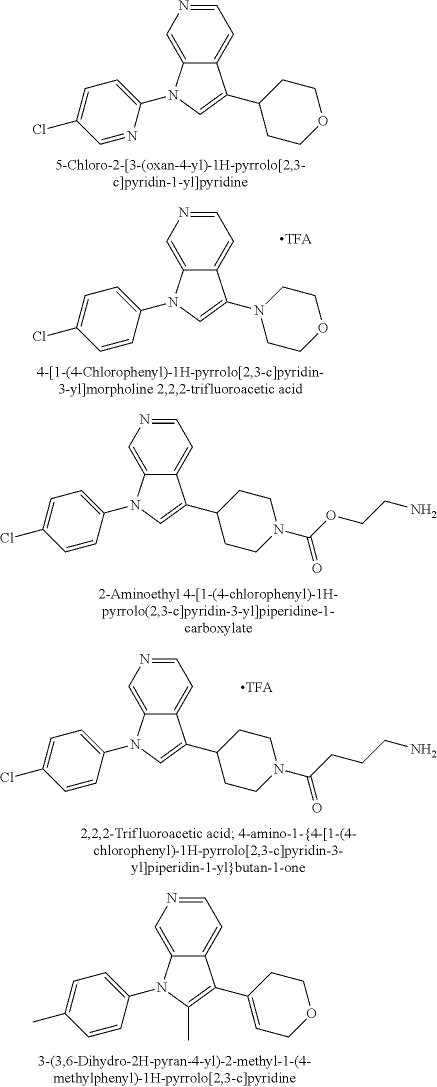 [SMILES: Clc1ccc(-n2cc(C3CCOCC3)c3ccncc32)nc1 ; Clc1ccc(-n2cc(N3CCOCC3)c3ccncc32)cc1 ; NCCOC(=O)N1CCC(c2cn(-c3ccc(Cl)cc3)c3cnccc23)CC1 ; NCCCC(=O)N1CCC(c2cn(-c3ccc(Cl)cc3)c3cnccc23)CC1 ; Cc1ccc(-n2c(C)c(C3=CCOCC3)c3ccncc32)cc1]

Further specific VAP-1 compounds include the Examples of WO2013/037411, namely:

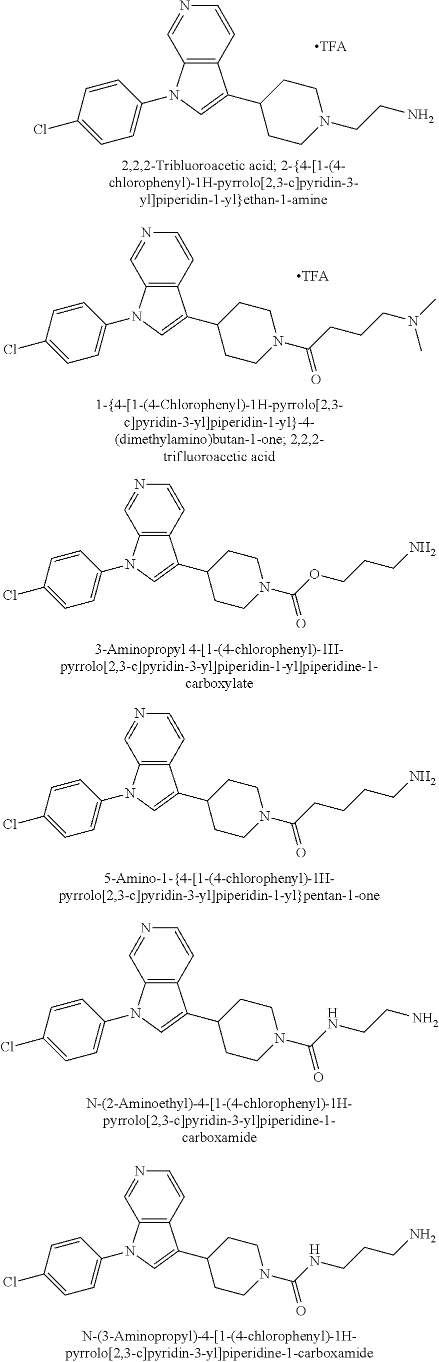 [SMILES: NCCN1CCC(c2cn(-c3ccc(Cl)cc3)c3cnccc23)CC1 ; CN(C)CCCC(=O)N1CCC(c2cn(-c3ccc(Cl)cc3)c3cnccc23)CC1 ; NCCCOC(=O)N1CCC(c2cn(-c3ccc(Cl)cc3)c3cnccc23)CC1 ; NCCCCC(=O)N1CCC(c2cn(-c3ccc(Cl)cc3)c3cnccc23)CC1 ; NCCNC(=O)N1CCC(c2cn(-c3ccc(Cl)cc3)c3cnccc23)CC1 ; NCCCNC(=O)N1CCC(c2cn(-c3ccc(Cl)cc3)c3cnccc23)CC1]

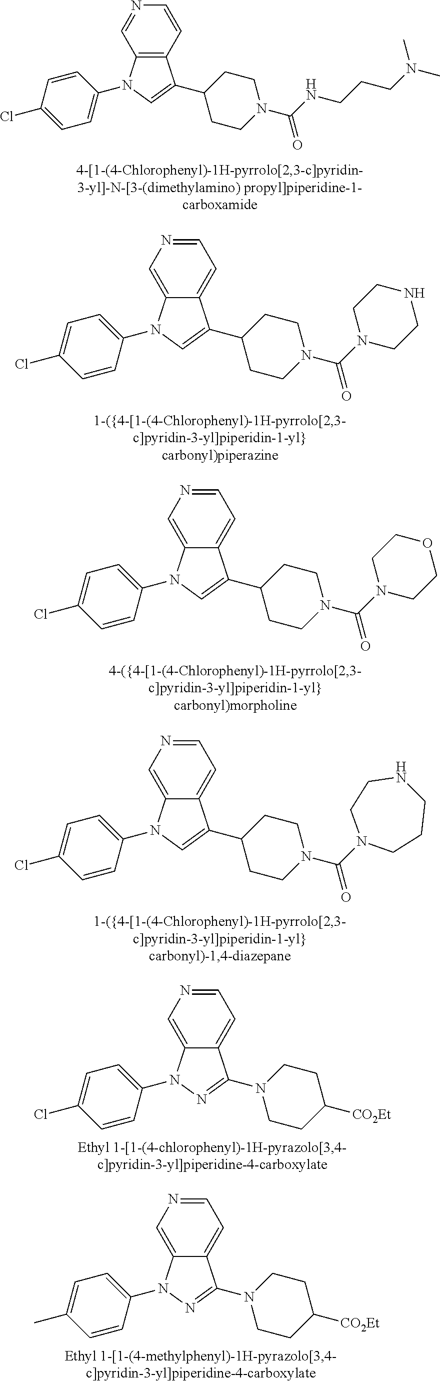

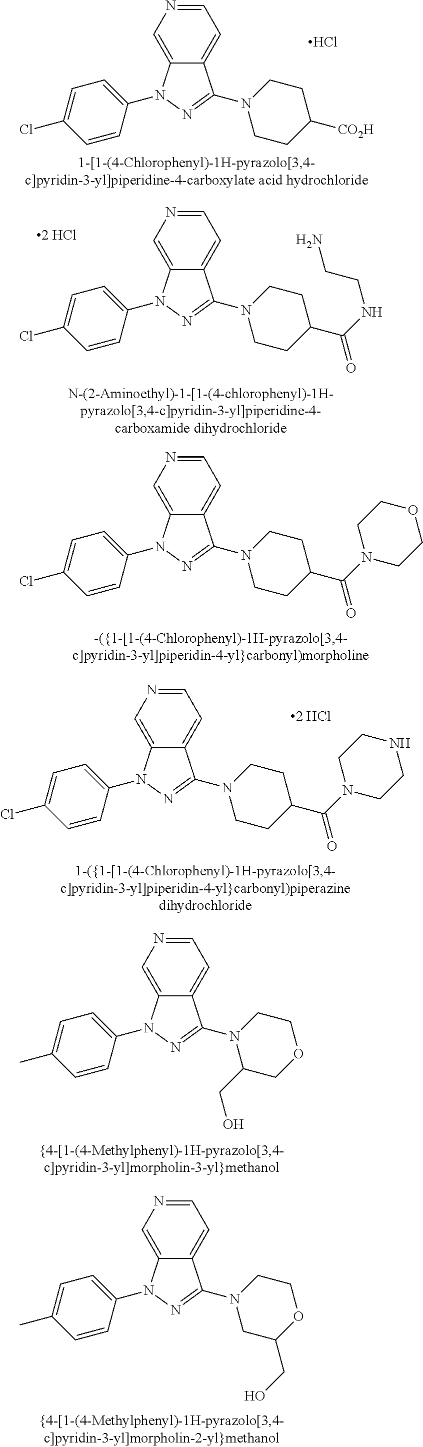

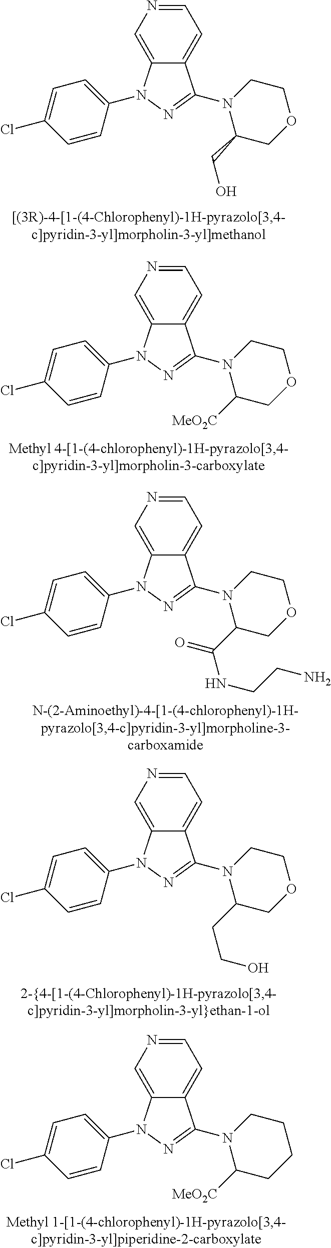 [SMILES: OC[C@@H]1COCCN1c1nn(-c2ccc(Cl)cc2)c2cnccc12 ; COC(=O)C1COCCN1c1nn(-c2ccc(Cl)cc2)c2cnccc12 ; NCCNC(=O)C1COCCN1c1nn(-c2ccc(Cl)cc2)c2cnccc12 ; OCCC1COCCN1c1nn(-c2ccc(Cl)cc2)c2cnccc12 ; COC(=O)C1CCCCN1c1nn(-c2ccc(Cl)cc2)c2cnccc12]

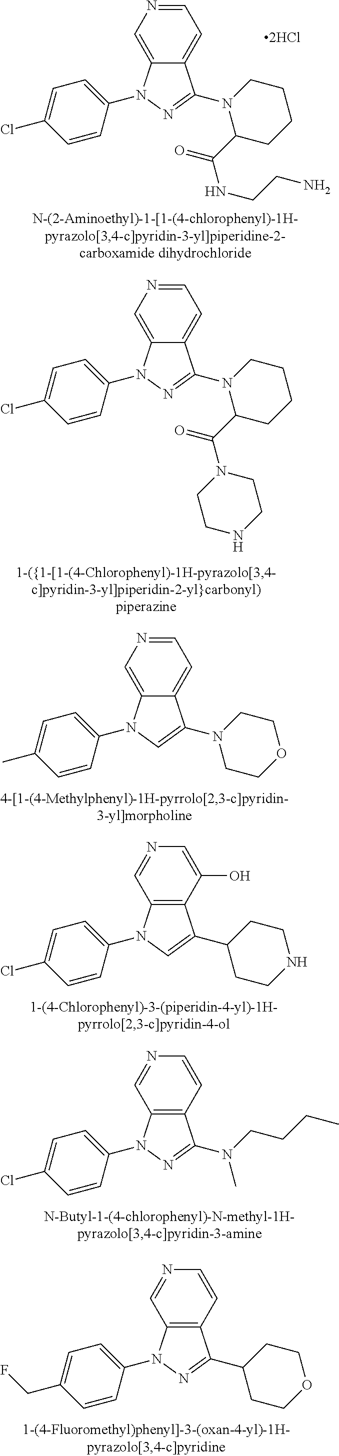 [SMILES: NCCNC(=O)C1CCCCN1c1nn(-c2ccc(Cl)cc2)c2cnccc12 ; O=C(C1CCCCN1c1nn(-c2ccc(Cl)cc2)c2cnccc12)N1CCNCC1 ; Cc1ccc(-n2cc(N3CCOCC3)c3ccncc32)cc1 ; Oc1cncc2c1c(C1CCNCC1)cn2-c1ccc(Cl)cc1 ; CCCCN(C)c1nn(-c2ccc(Cl)cc2)c2cnccc12 ; FCc1ccc(-n2nc(C3CCOCC3)c3ccncc32)cc1]

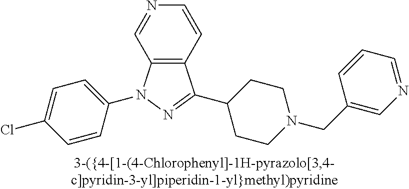 [SMILES: Clc1ccc(-n2nc(C3CCN(Cc4cccnc4)CC3)c3ccncc32)cc1]

Further specific examples of VAP-1 compounds include the Examples of WO2013/038189, namely:

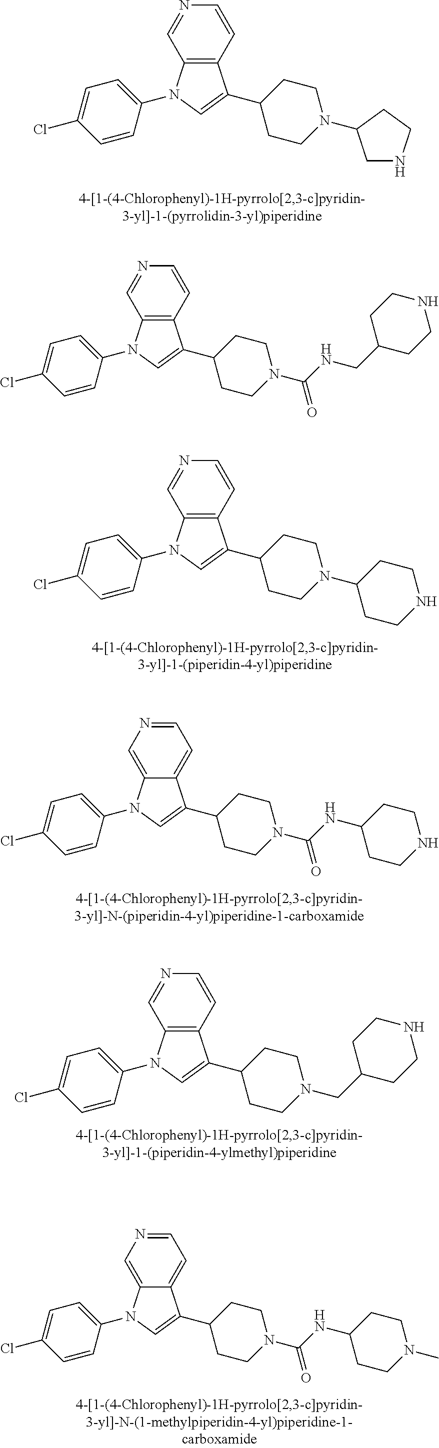 [SMILES: Clc1ccc(-n2cc(C3CCN(C4CCNC4)CC3)c3ccncc32)cc1 ; O=C(NCC1CCNCC1)N1CCC(c2cn(-c3ccc(Cl)cc3)c3cnccc23)CC1 ; Clc1ccc(-n2cc(C3CCN(C4CCNCC4)CC3)c3ccncc32)cc1 ; O=C(NC1CCNCC1)N1CCC(c2cn(-c3ccc(Cl)cc3)c3cnccc23)CC1 ; Clc1ccc(-n2cc(C3CCN(CC4CCNCC4)CC3)c3ccncc32)cc1 ; CN1CCC(NC(=O)N2CCC(c3cn(-c4ccc(Cl)cc4)c4cnccc34)CC2)CC1]

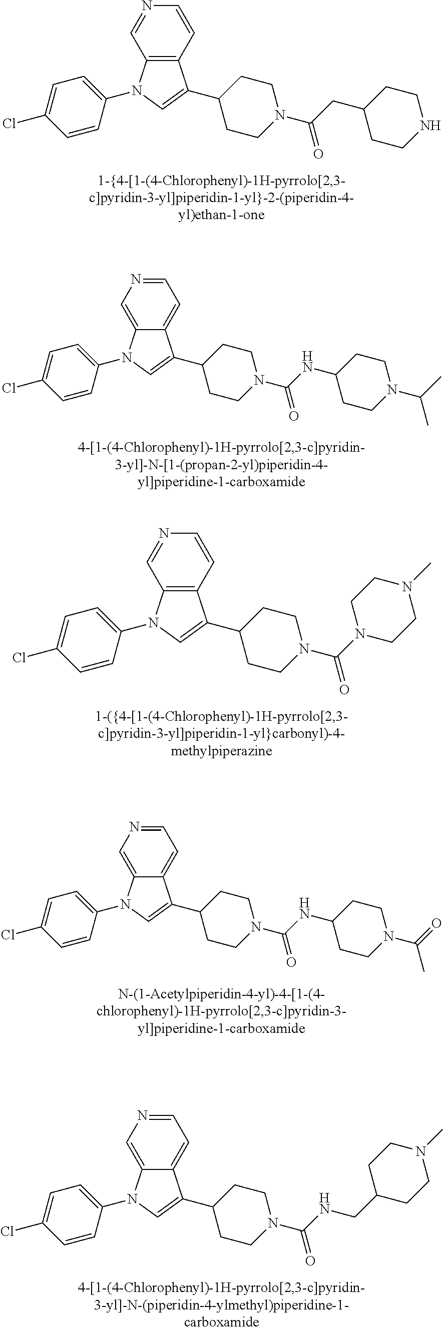 [SMILES: O=C(CC1CCNCC1)N1CCC(c2cn(-c3ccc(Cl)cc3)c3cnccc23)CC1 ; CC(C)N1CCC(NC(=O)N2CCC(c3cn(-c4ccc(Cl)cc4)c4cnccc34)CC2)CC1 ; CN1CCN(C(=O)N2CCC(c3cn(-c4ccc(Cl)cc4)c4cnccc34)CC2)CC1 ; CC(=O)N1CCC(NC(=O)N2CCC(c3cn(-c4ccc(Cl)cc4)c4cnccc34)CC2)CC1 ; CN1CCC(CNC(=O)N2CCC(c3cn(-c4ccc(Cl)cc4)c4cnccc34)CC2)CC1]

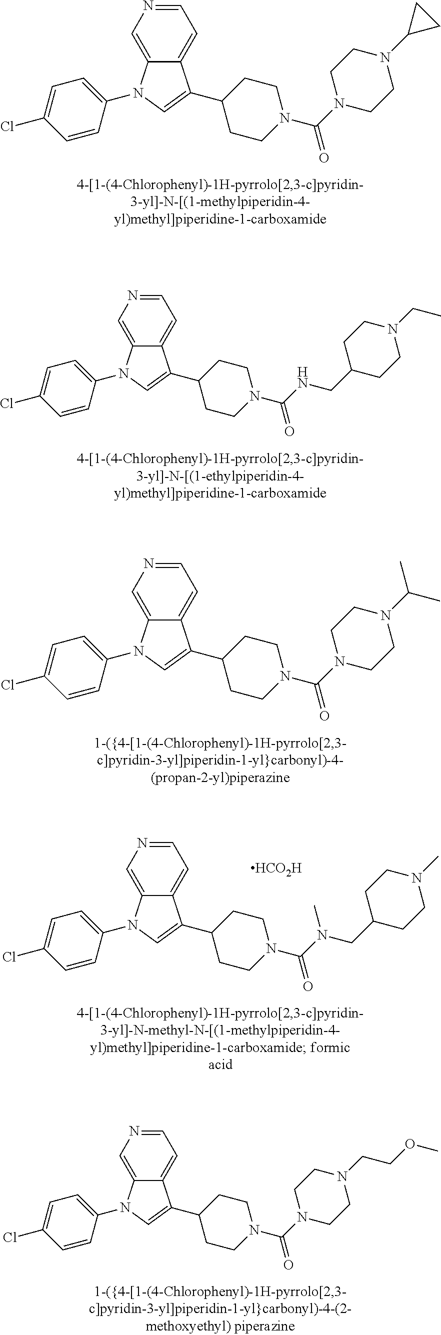 [SMILES: O=C(N1CCC(c2cn(-c3ccc(Cl)cc3)c3cnccc23)CC1)N1CCN(C2CC2)CC1 ; CCN1CCC(CNC(=O)N2CCC(c3cn(-c4ccc(Cl)cc4)c4cnccc34)CC2)CC1 ; CC(C)N1CCN(C(=O)N2CCC(c3cn(-c4ccc(Cl)cc4)c4cnccc34)CC2)CC1 ; CN1CCC(CN(C)C(=O)N2CCC(c3cn(-c4ccc(Cl)cc4)c4cnccc34)CC2)CC1 ; "·HCO2H" ; COCCN1CCN(C(=O)N2CCC(c3cn(-c4ccc(Cl)cc4)c4cnccc34)CC2)CC1]

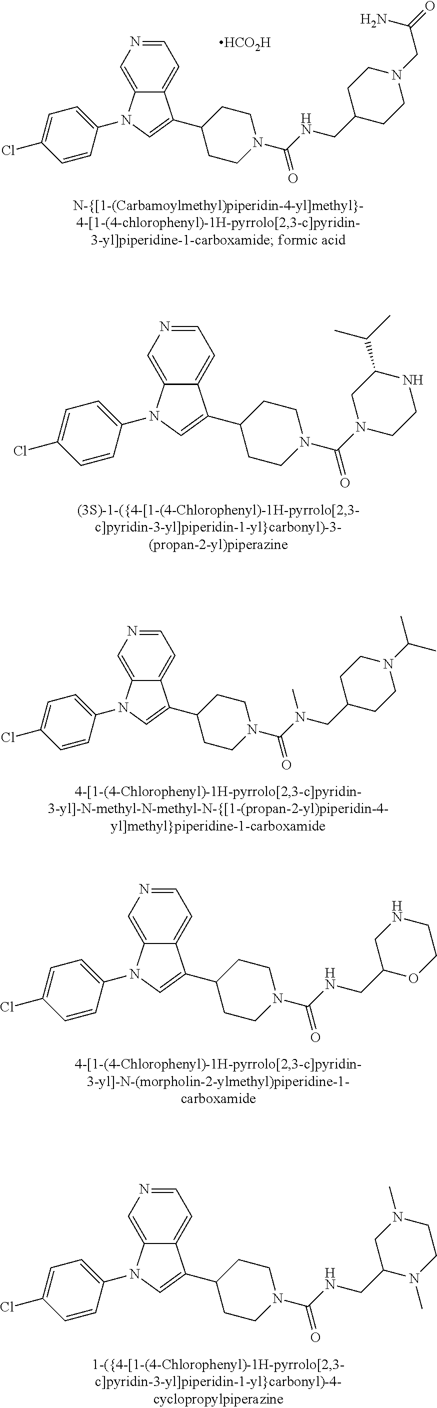 [SMILES: NC(=O)CN1CCC(CNC(=O)N2CCC(c3cn(-c4ccc(Cl)cc4)c4cnccc34)CC2)CC1 ; "·HCO2H" ; CC(C)[C@H]1CN(C(=O)N2CCC(c3cn(-c4ccc(Cl)cc4)c4cnccc34)CC2)CCN1 ; CC(C)N1CCC(CN(C)C(=O)N2CCC(c3cn(-c4ccc(Cl)cc4)c4cnccc34)CC2)CC1 ; O=C(NCC1CNCCO1)N1CCC(c2cn(-c3ccc(Cl)cc3)c3cnccc23)CC1 ; CN1CCN(C)C(CNC(=O)N2CCC(c3cn(-c4ccc(Cl)cc4)c4cnccc34)CC2)C1]

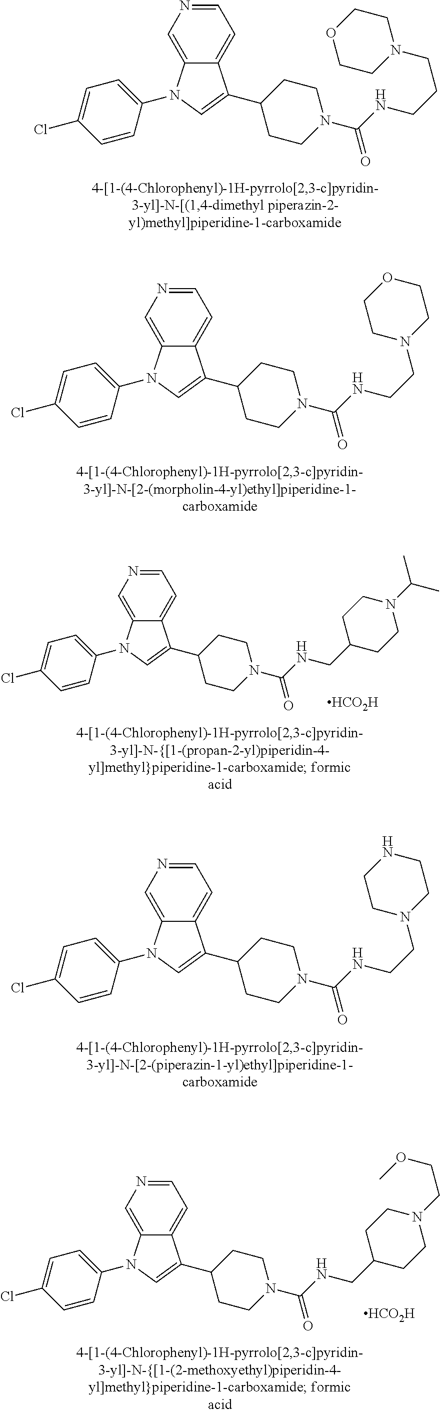

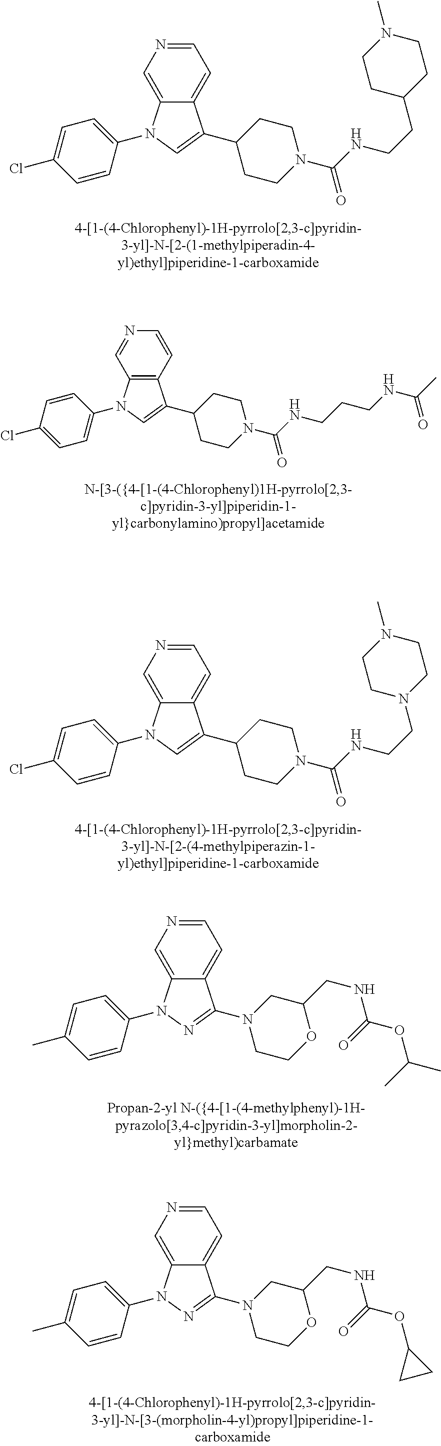

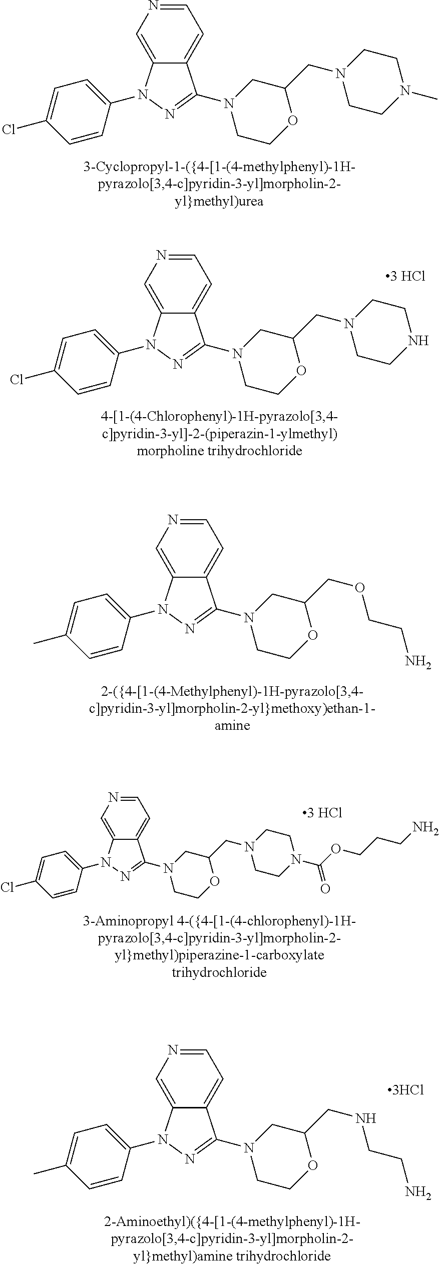

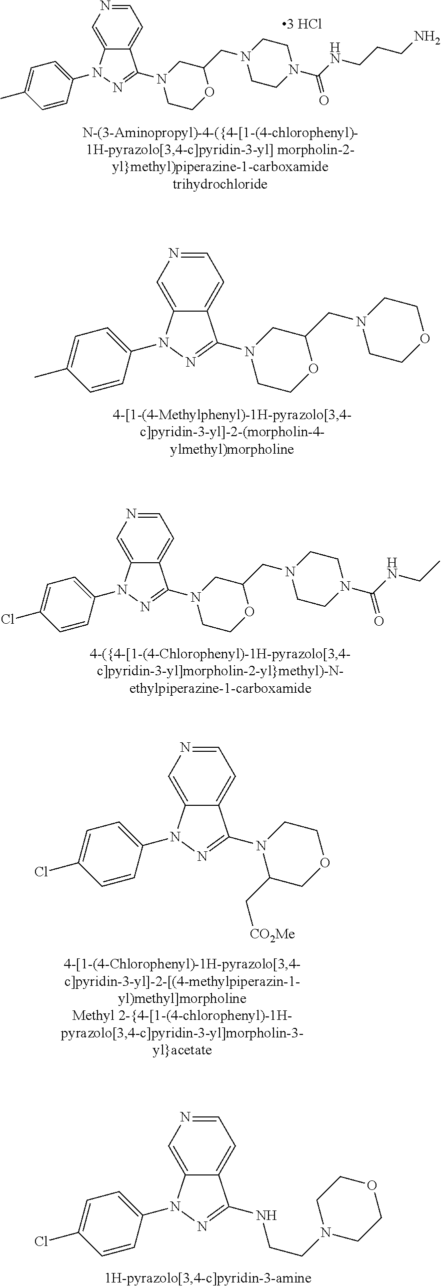 [SMILES: Cc1ccc(-n2nc(N3CCOC(CN4CCN(C(=O)NCCCN)CC4)C3)c3ccncc32)cc1 ; Cc1ccc(-n2nc(N3CCOC(CN4CCOCC4)C3)c3ccncc32)cc1 ; CCNC(=O)N1CCN(CC2CN(c3nn(-c4ccc(Cl)cc4)c4cnccc34)CCO2)CC1 ; COC(=O)CC1COCCN1c1nn(-c2ccc(Cl)cc2)c2cnccc12 ; Clc1ccc(-n2nc(NCCN3CCOCC3)c3ccncc32)cc1]

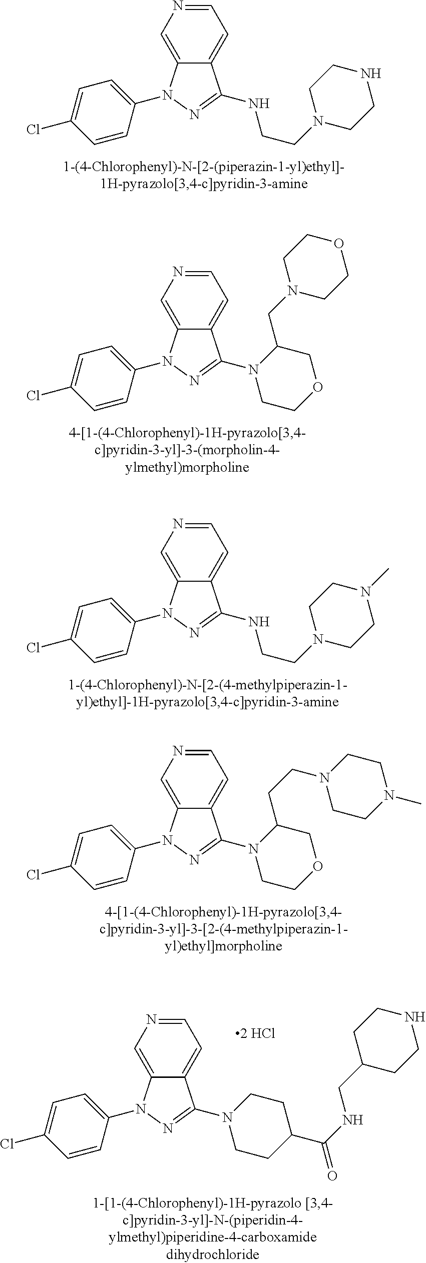 [SMILES: Clc1ccc(-n2nc(NCCN3CCNCC3)c3ccncc32)cc1 ; Clc1ccc(-n2nc(N3CCOCC3CN3CCOCC3)c3ccncc32)cc1 ; CN1CCN(CCNc2nn(-c3ccc(Cl)cc3)c3cnccc23)CC1 ; CN1CCN(CCC2COCCN2c2nn(-c3ccc(Cl)cc3)c3cnccc23)CC1 ; O=C(NCC1CCNCC1)C1CCN(c2nn(-c3ccc(Cl)cc3)c3cnccc23)CC1]

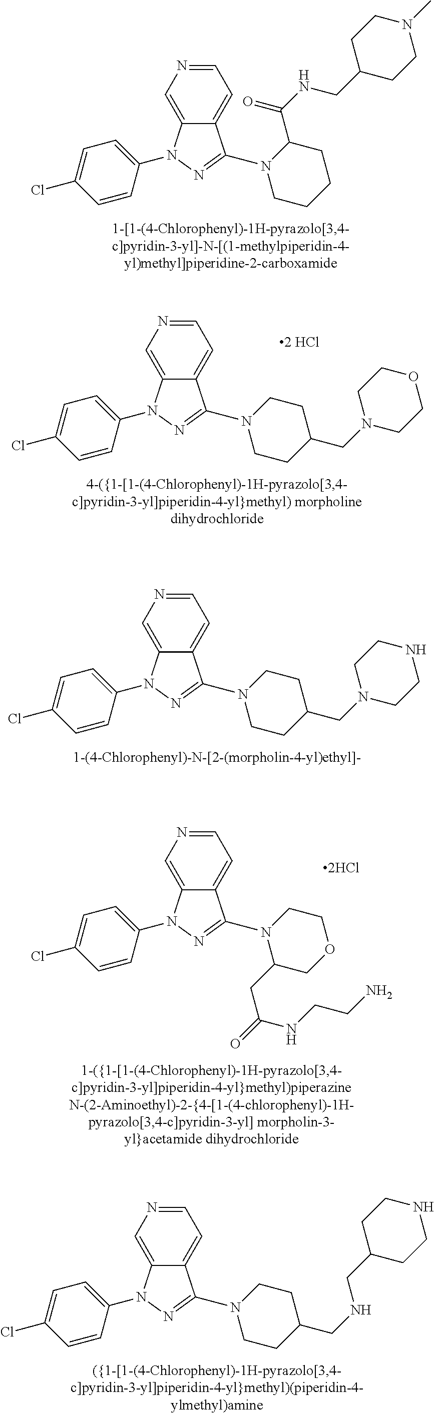 [SMILES: CN1CCC(CNC(=O)C2CCCCN2c2nn(-c3ccc(Cl)cc3)c3cnccc23)CC1 ; Clc1ccc(-n2nc(N3CCC(CN4CCOCC4)CC3)c3ccncc32)cc1 ; Clc1ccc(-n2nc(N3CCC(CN4CCNCC4)CC3)c3ccncc32)cc1 ; NCCNC(=O)CC1COCCN1c1nn(-c2ccc(Cl)cc2)c2cnccc12 ; Clc1ccc(-n2nc(N3CCC(CNCC4CCNCC4)CC3)c3ccncc32)cc1]

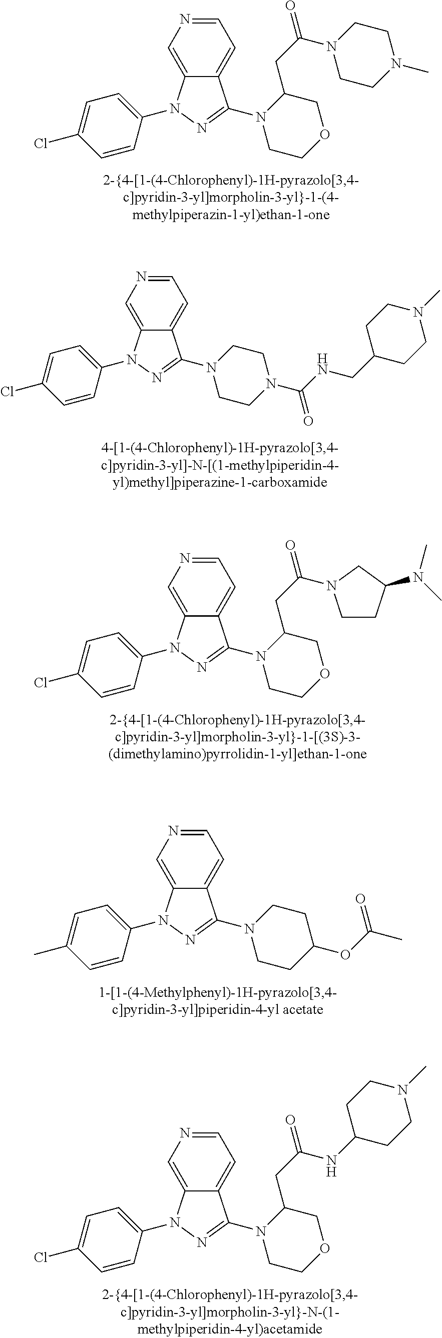

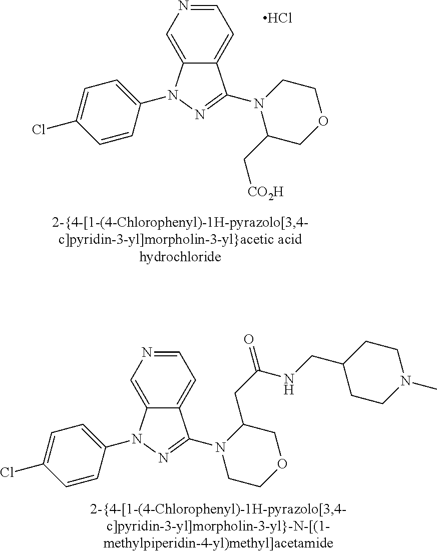 [SMILES: O=C(O)CC1COCCN1c1nn(-c2ccc(Cl)cc2)c2cnccc12 ; Cl ; CN1CCC(CNC(=O)CC2COCCN2c2nn(-c3ccc(Cl)cc3)c3cnccc23)CC1]

Specific examples of inhibitors of VAP-1 include the compounds speficially disclosed as Examples in WO 2010/031791, namely:

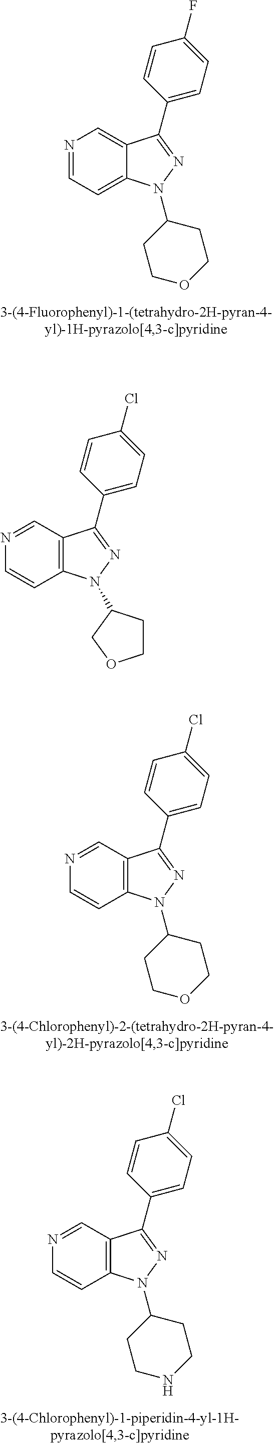

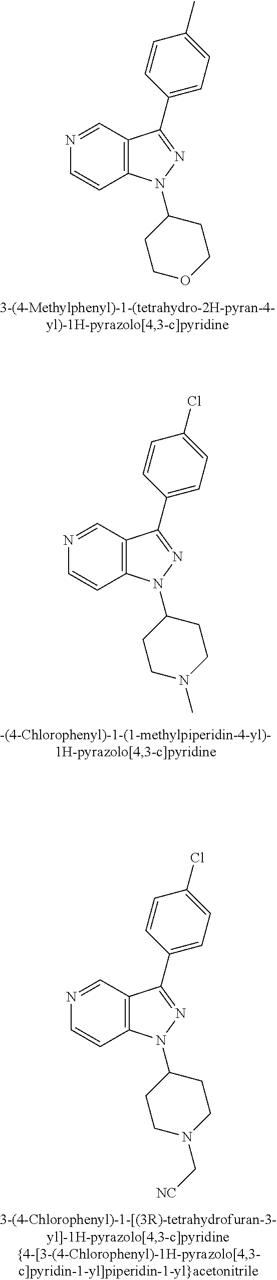 [SMILES: Cc1ccc(-c2nn(C3CCOCC3)c3ccncc23)cc1 ; CN1CCC(n2nc(-c3ccc(Cl)cc3)c3cnccc32)CC1 ; N#CCN1CCC(n2nc(-c3ccc(Cl)cc3)c3cnccc32)CC1]

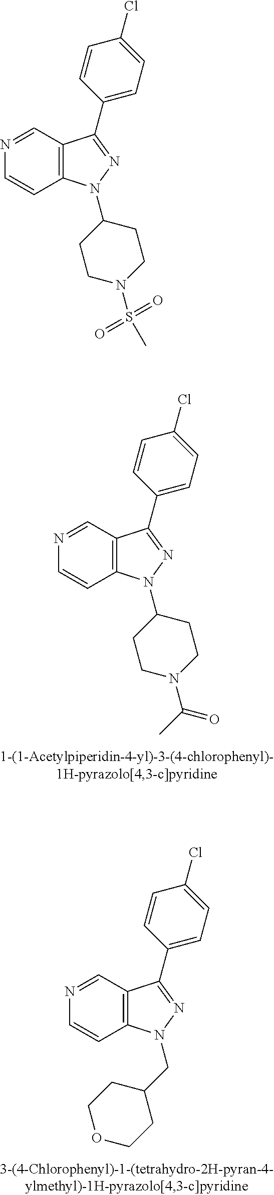 [SMILES: CS(=O)(=O)N1CCC(n2nc(-c3ccc(Cl)cc3)c3cnccc32)CC1 ; CC(=O)N1CCC(n2nc(-c3ccc(Cl)cc3)c3cnccc32)CC1 ; Clc1ccc(-c2nn(CC3CCOCC3)c3ccncc23)cc1]

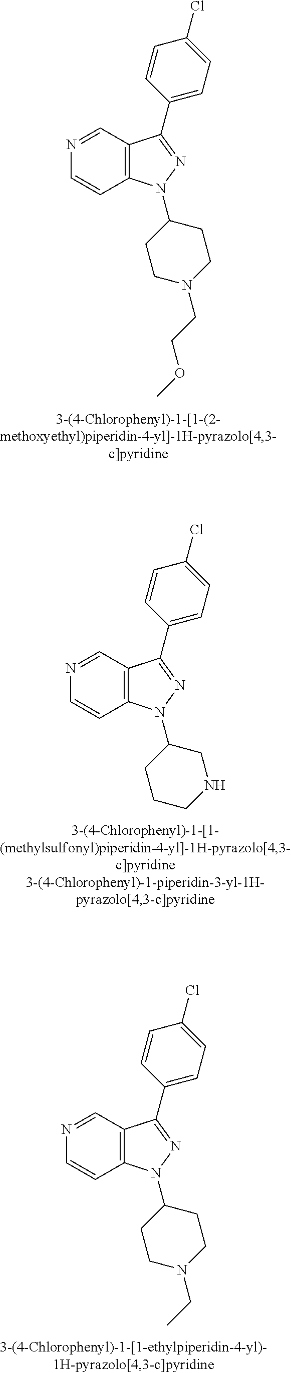 [SMILES: COCCN1CCC(n2nc(-c3ccc(Cl)cc3)c3cnccc32)CC1 ; Clc1ccc(-c2nn(C3CCCNC3)c3ccncc23)cc1 ; CCN1CCC(n2nc(-c3ccc(Cl)cc3)c3cnccc32)CC1]

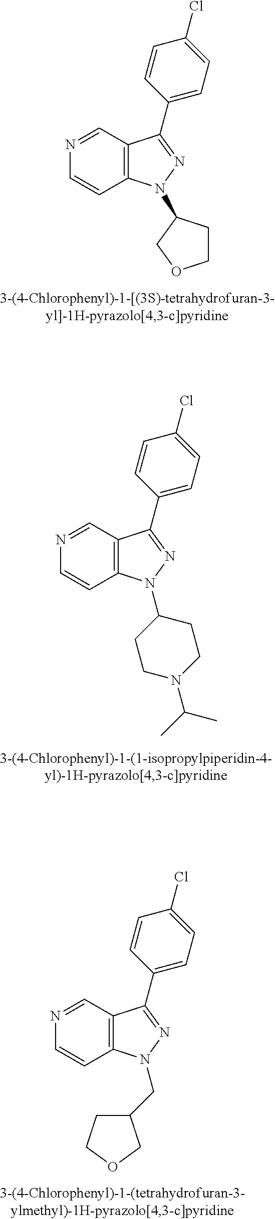 [SMILES: Clc1ccc(-c2nn([C@H]3CCOC3)c3ccncc23)cc1 ; CC(C)N1CCC(n2nc(-c3ccc(Cl)cc3)c3cnccc32)CC1 ; Clc1ccc(-c2nn(CC3CCOC3)c3ccncc23)cc1]

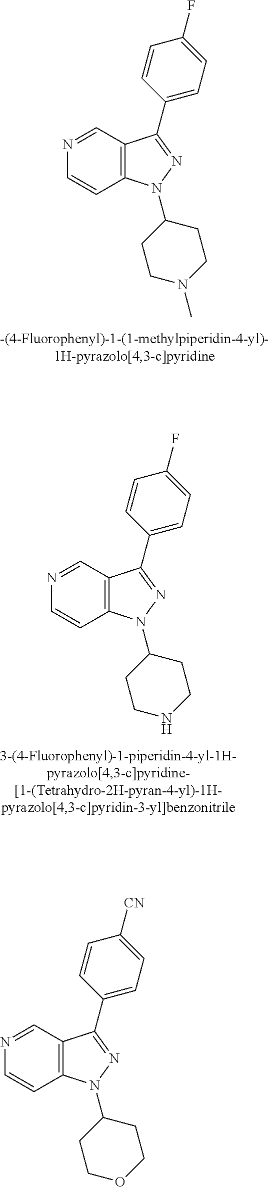 [SMILES: CN1CCC(n2nc(-c3ccc(F)cc3)c3cnccc32)CC1 ; Fc1ccc(-c2nn(C3CCNCC3)c3ccncc23)cc1 ; N#Cc1ccc(-c2nn(C3CCOCC3)c3ccncc23)cc1]

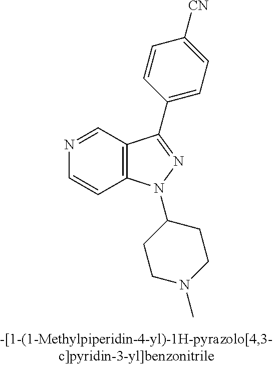 [SMILES: CN1CCC(n2nc(-c3ccc(C#N)cc3)c3cnccc32)CC1]

Specific examples of inhibitors of VAP-1 include the compounds speficially disclosed as Examples in WO 2010/064020, namely:

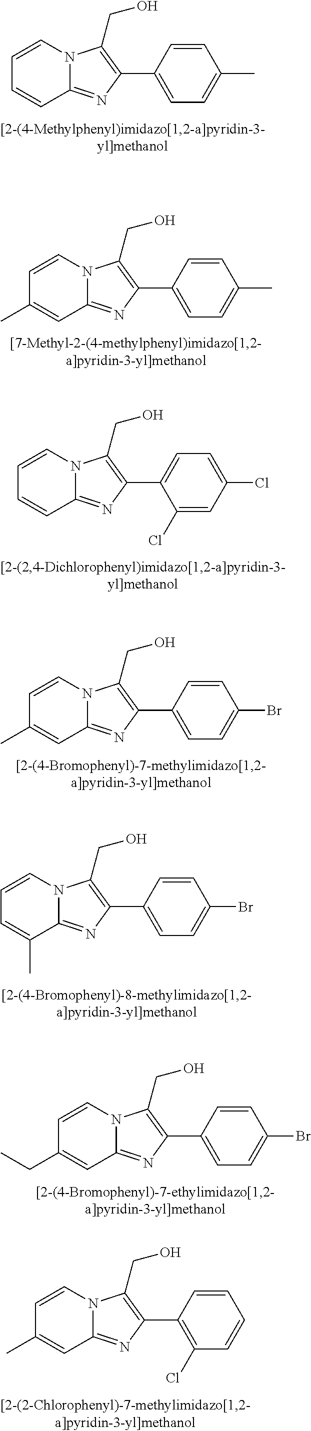

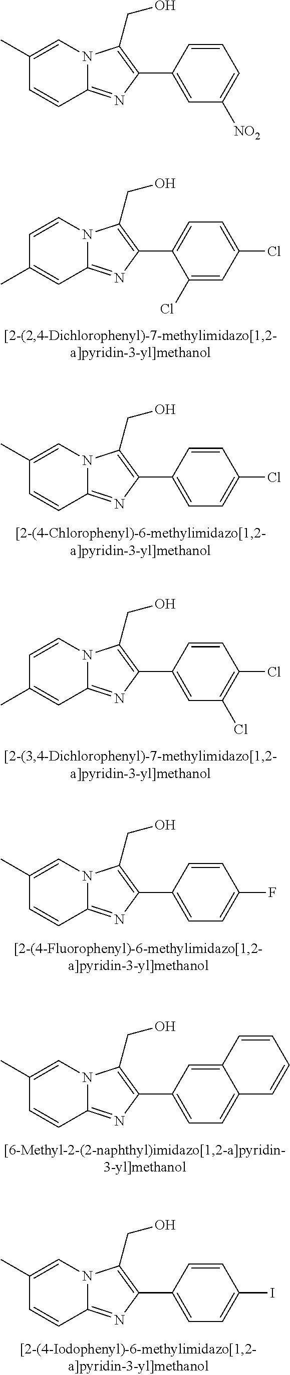

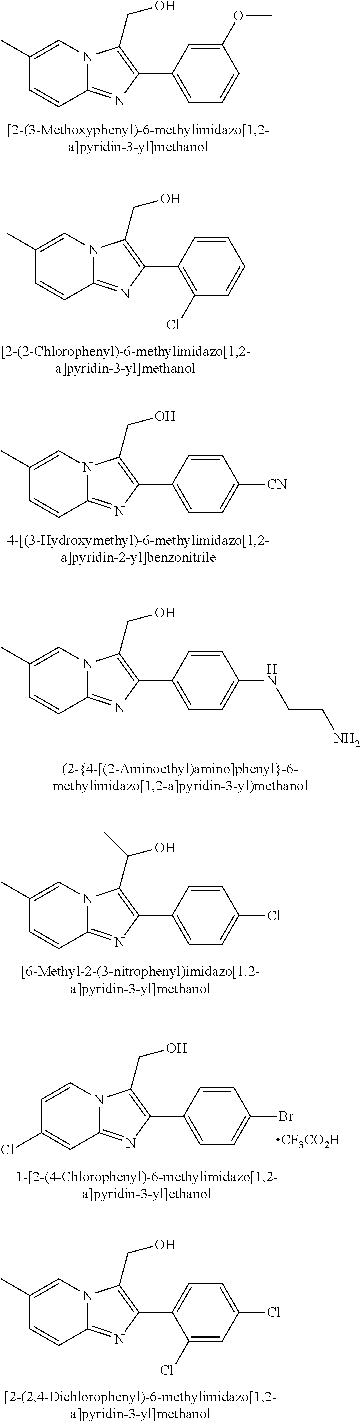

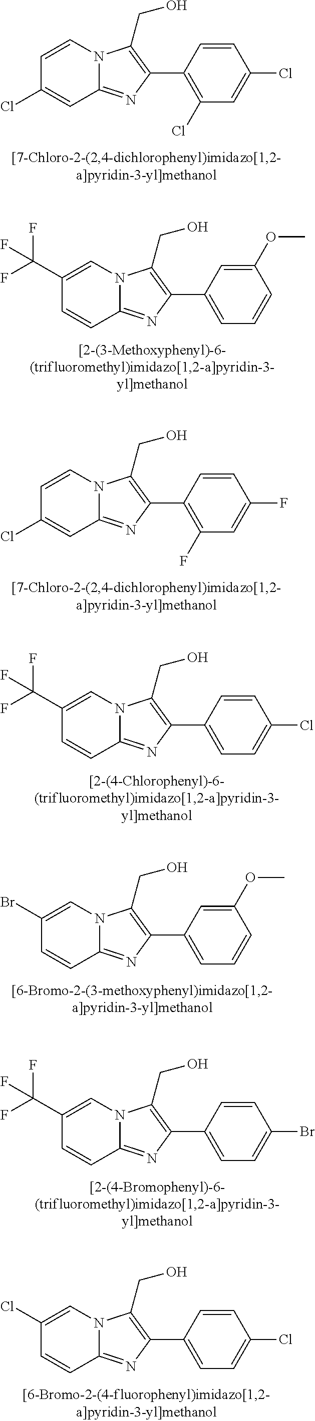

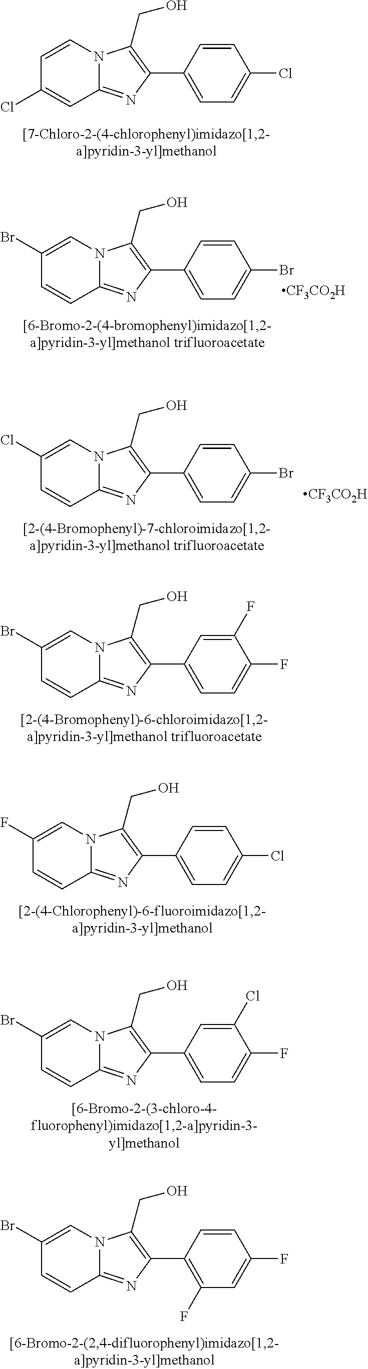

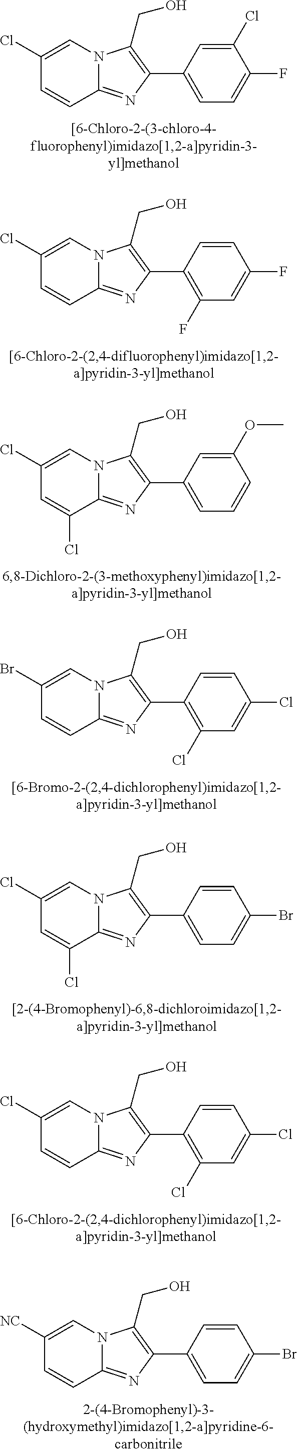

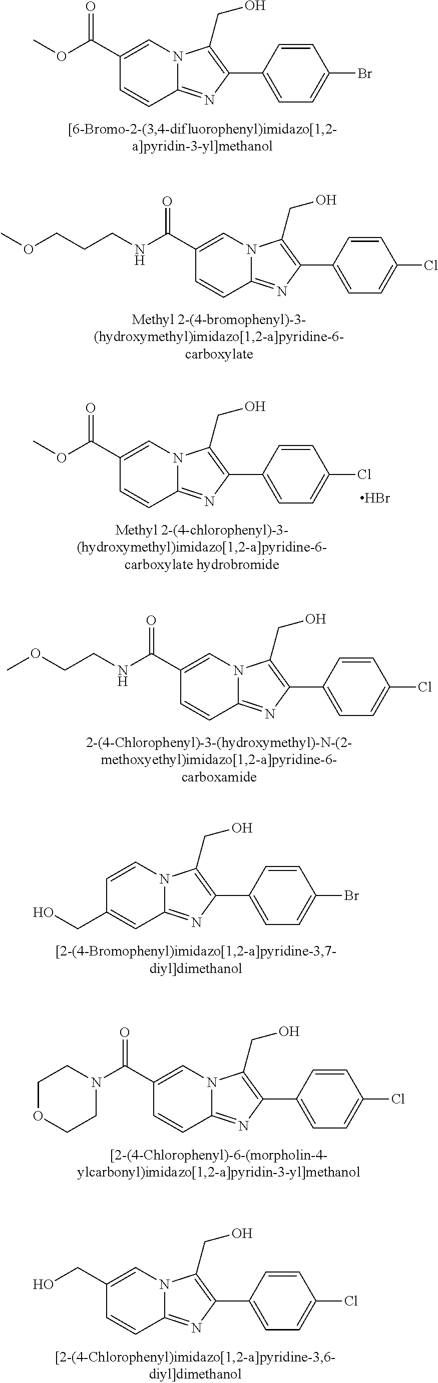

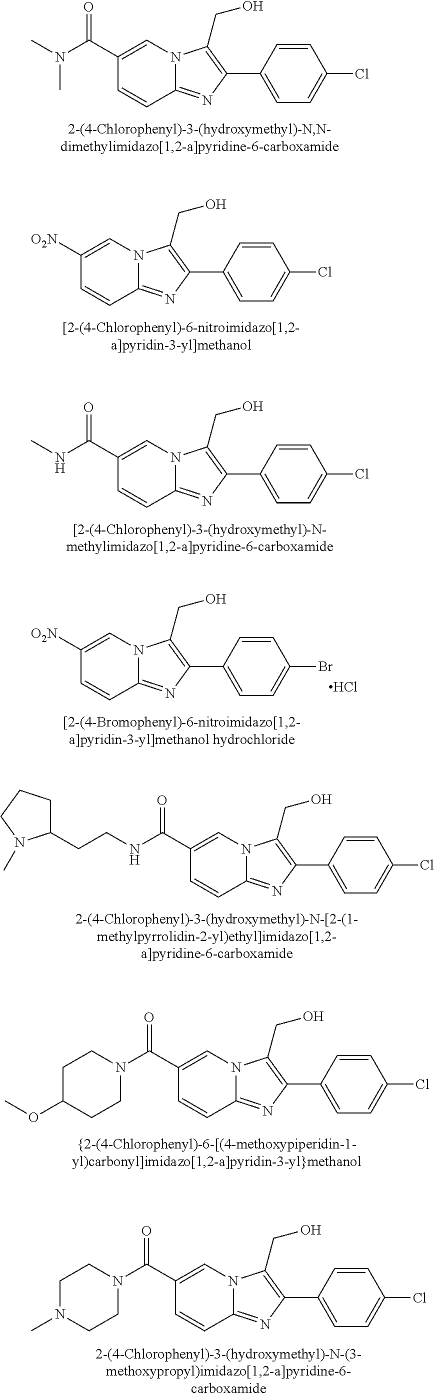

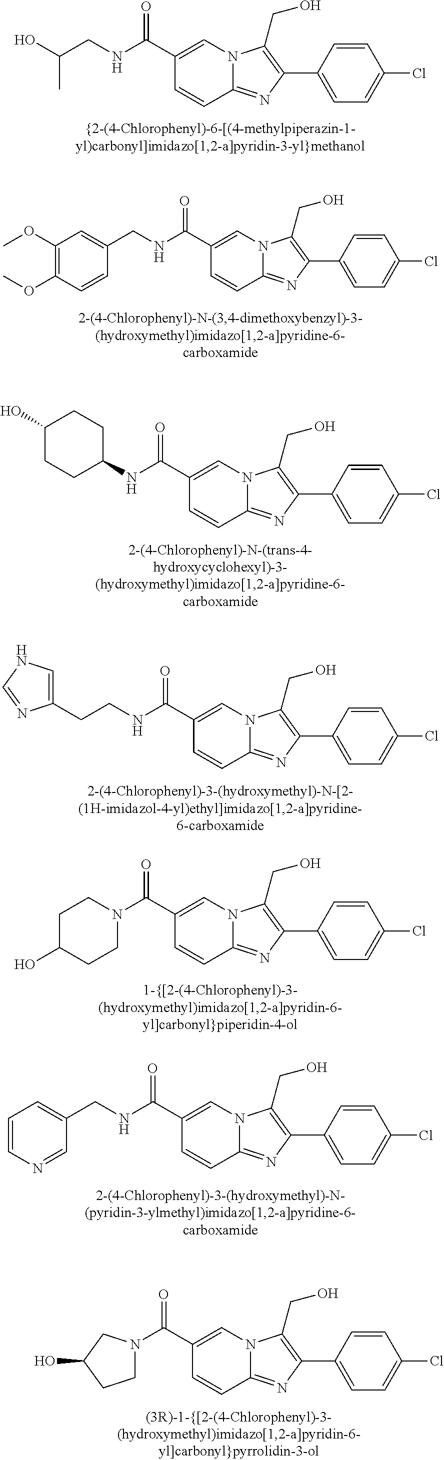

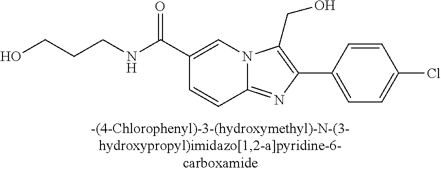 [SMILES: O=C(NCCCO)c1ccc2nc(-c3ccc(Cl)cc3)c(CO)n2c1]

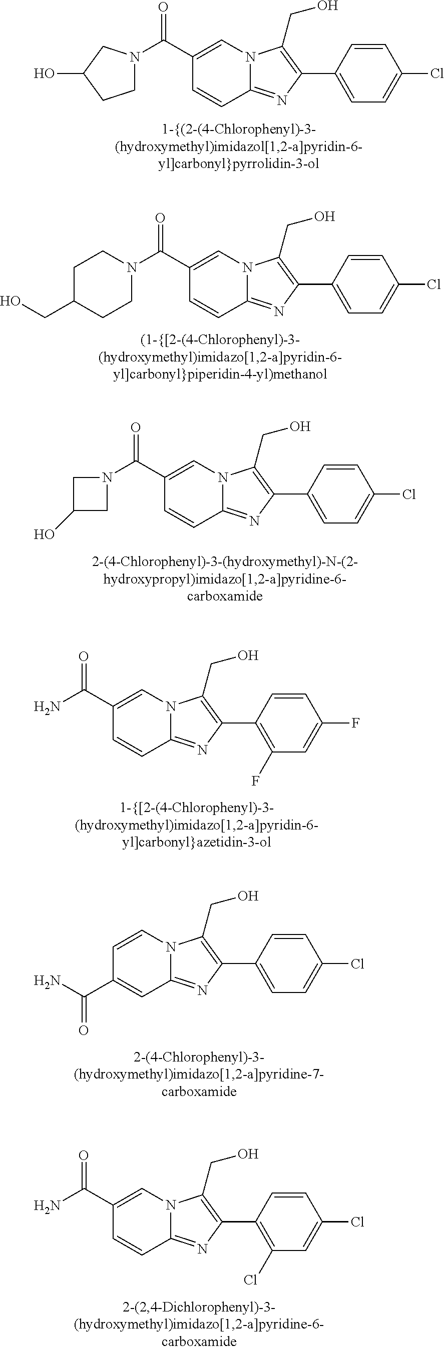 [SMILES: O=C(c1ccc2nc(-c3ccc(Cl)cc3)c(CO)n2c1)N1CCC(O)C1 ; O=C(c1ccc2nc(-c3ccc(Cl)cc3)c(CO)n2c1)N1CCC(CO)CC1 ; O=C(c1ccc2nc(-c3ccc(Cl)cc3)c(CO)n2c1)N1CC(O)C1 ; NC(=O)c1ccc2nc(-c3ccc(F)cc3F)c(CO)n2c1 ; NC(=O)c1ccn2c(CO)c(-c3ccc(Cl)cc3)nc2c1 ; NC(=O)c1ccc2nc(-c3ccc(Cl)cc3Cl)c(CO)n2c1]

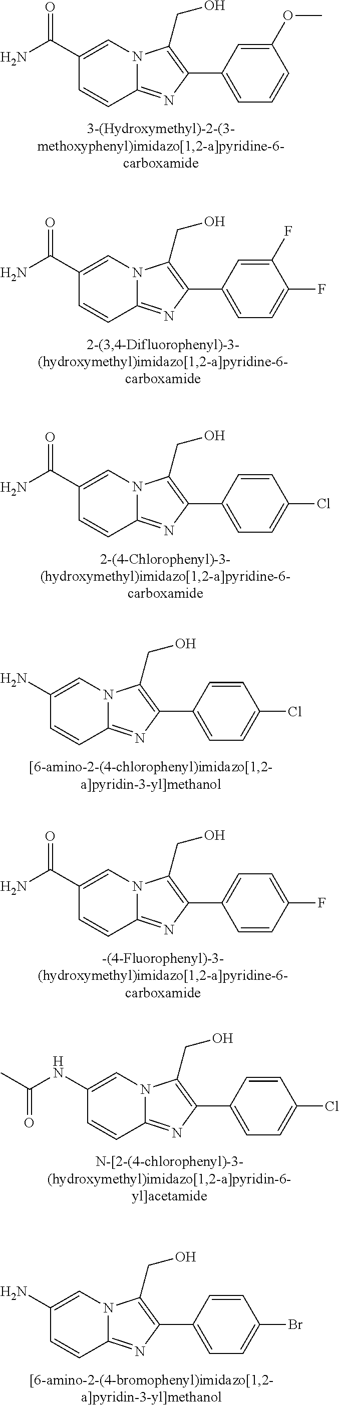

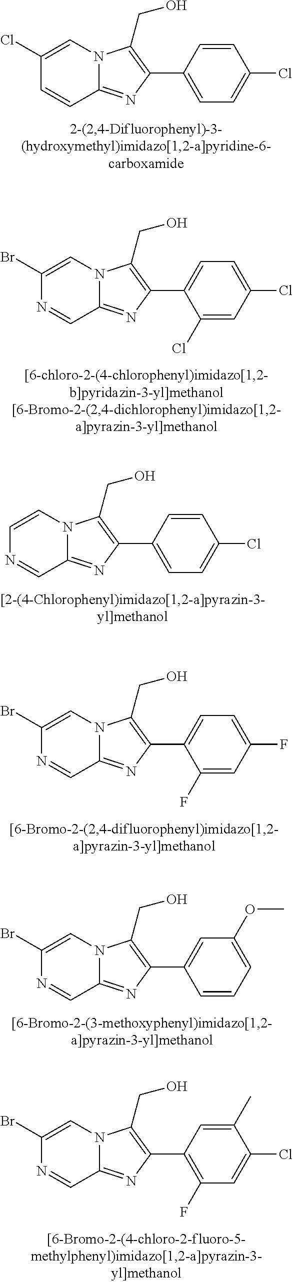 [SMILES: OCc1c(-c2ccc(Cl)cc2)nc2ccc(Cl)cn12 ; OCc1c(-c2ccc(Cl)cc2Cl)nc2cnc(Br)cn12 ; OCc1c(-c2ccc(Cl)cc2)nc2cnccn12 ; OCc1c(-c2ccc(F)cc2F)nc2cnc(Br)cn12 ; COc1cccc(-c2nc3cnc(Br)cn3c2CO)c1 ; Cc1cc(-c2nc3cnc(Br)cn3c2CO)c(F)cc1Cl]

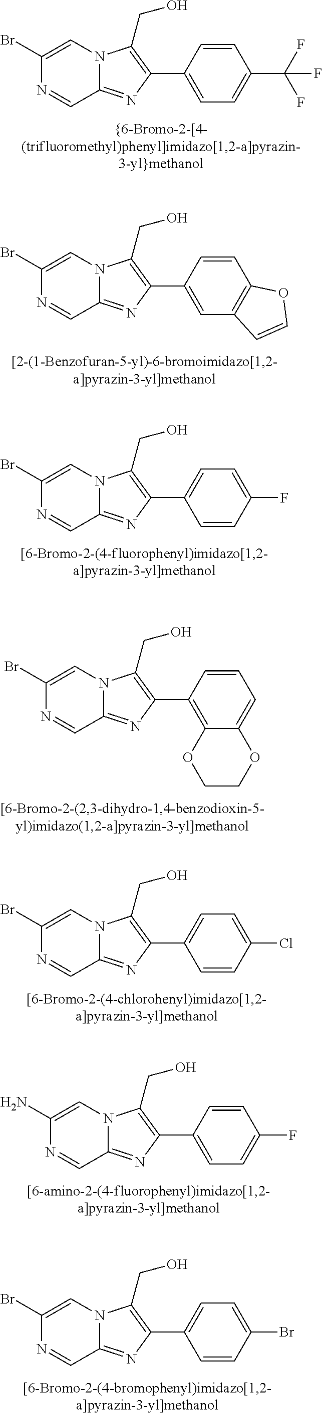

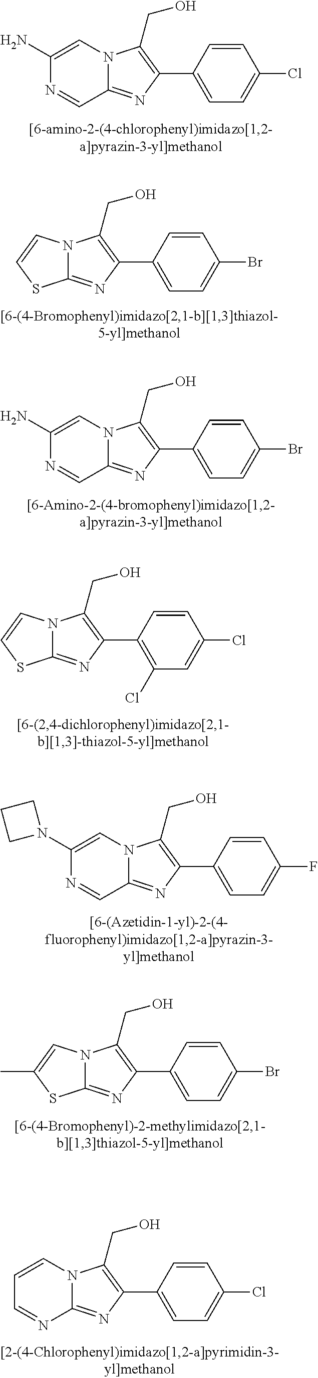

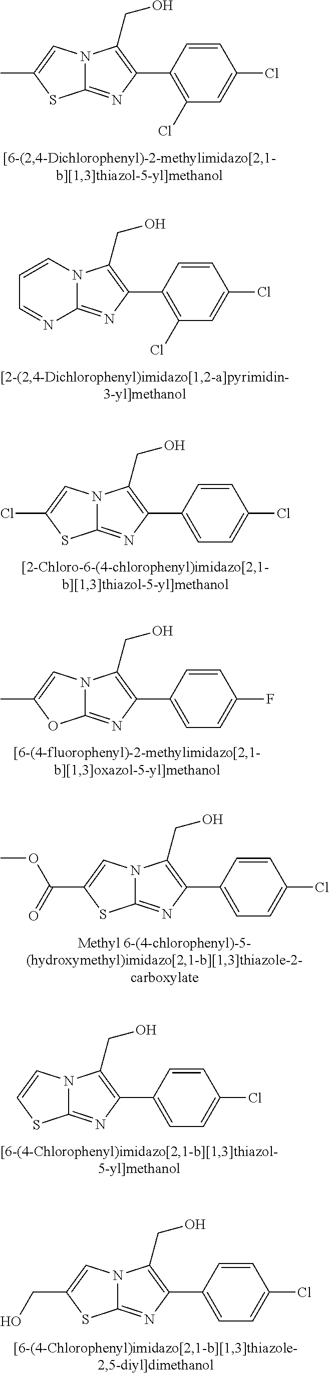

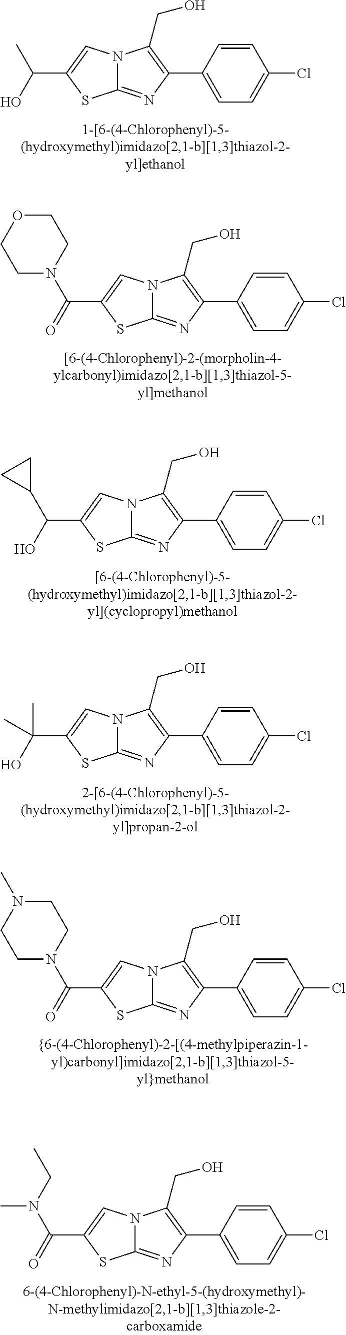 [SMILES: CC(O)c1cn2c(CO)c(-c3ccc(Cl)cc3)nc2s1 ; O=C(c1cn2c(CO)c(-c3ccc(Cl)cc3)nc2s1)N1CCOCC1 ; OCc1c(-c2ccc(Cl)cc2)nc2sc(C(O)C3CC3)cn12 ; CC(C)(O)c1cn2c(CO)c(-c3ccc(Cl)cc3)nc2s1 ; CN1CCN(C(=O)c2cn3c(CO)c(-c4ccc(Cl)cc4)nc3s2)CC1 ; CCN(C)C(=O)c1cn2c(CO)c(-c3ccc(Cl)cc3)nc2s1]

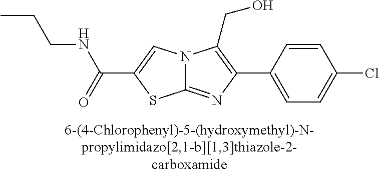 [SMILES: CCCNC(=O)c1cn2c(CO)c(-c3ccc(Cl)cc3)nc2s1]

Further specific Examples of VAP-1 compounds include:

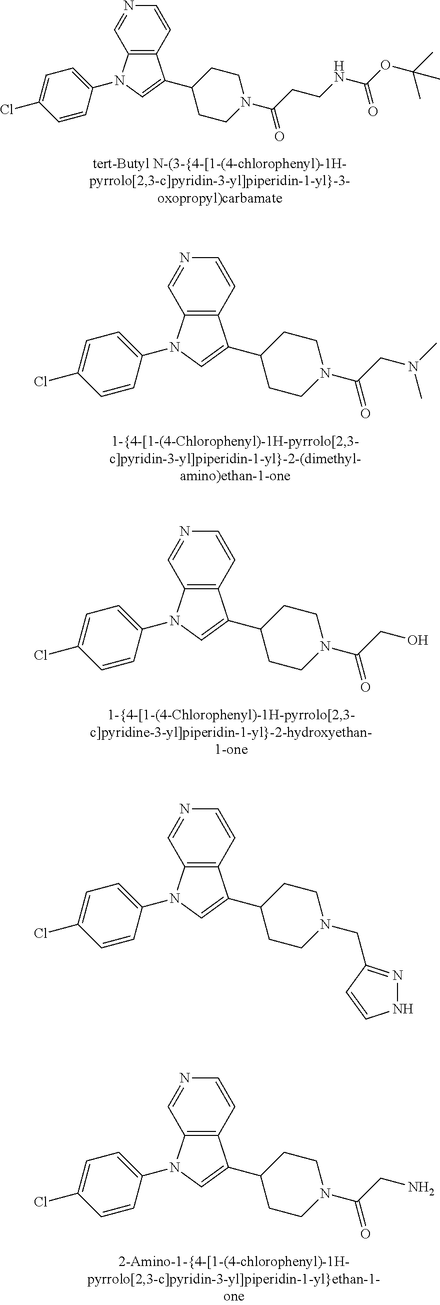 [SMILES: CC(C)(C)OC(=O)NCCC(=O)N1CCC(c2cn(-c3ccc(Cl)cc3)c3cnccc23)CC1 ; CN(C)CC(=O)N1CCC(c2cn(-c3ccc(Cl)cc3)c3cnccc23)CC1 ; O=C(CO)N1CCC(c2cn(-c3ccc(Cl)cc3)c3cnccc23)CC1 ; Clc1ccc(-n2cc(C3CCN(Cc4cc[nH]n4)CC3)c3ccncc32)cc1 ; NCC(=O)N1CCC(c2cn(-c3ccc(Cl)cc3)c3cnccc23)CC1]

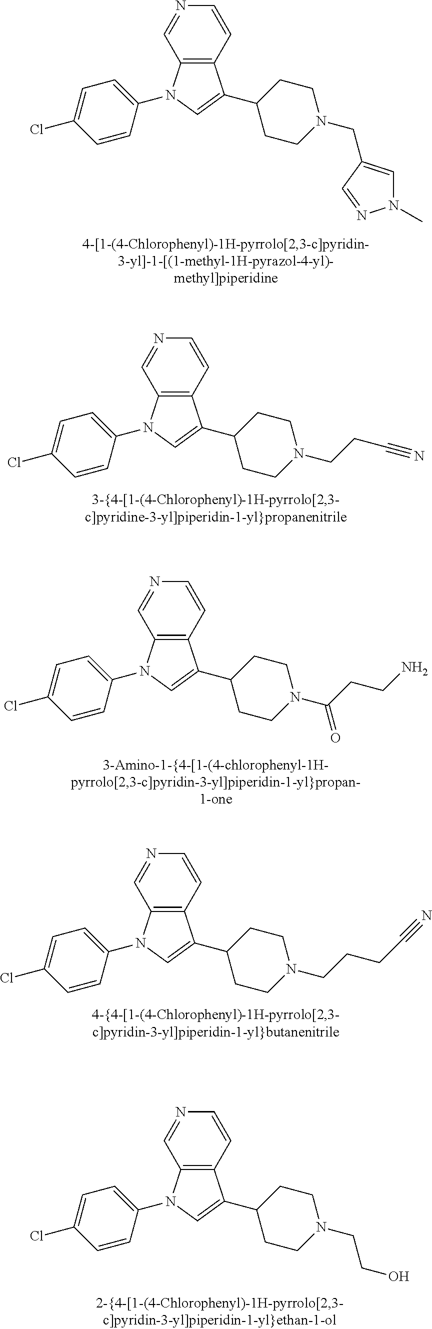 [SMILES: Cn1cc(CN2CCC(c3cn(-c4ccc(Cl)cc4)c4cnccc34)CC2)cn1 ; N#CCCN1CCC(c2cn(-c3ccc(Cl)cc3)c3cnccc23)CC1 ; NCCC(=O)N1CCC(c2cn(-c3ccc(Cl)cc3)c3cnccc23)CC1 ; N#CCCCN1CCC(c2cn(-c3ccc(Cl)cc3)c3cnccc23)CC1 ; OCCN1CCC(c2cn(-c3ccc(Cl)cc3)c3cnccc23)CC1]

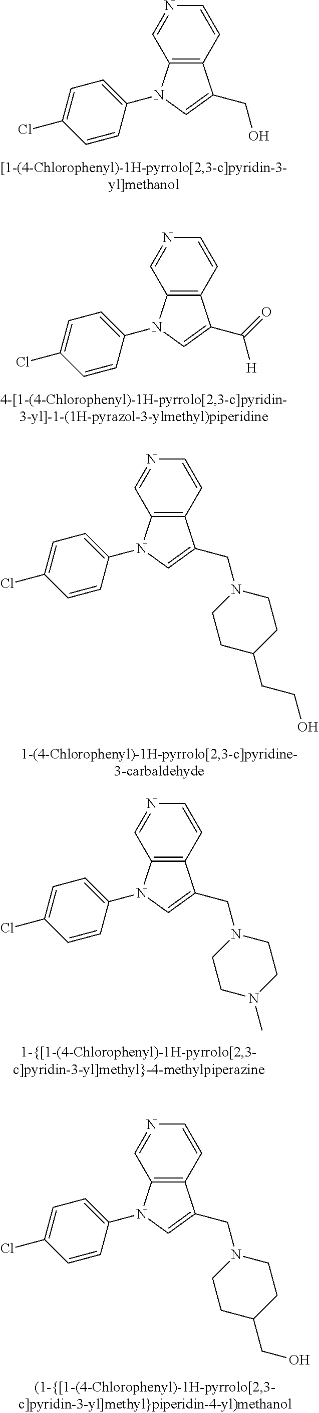 [SMILES: OCc1cn(-c2ccc(Cl)cc2)c2cnccc12 ; O=Cc1cn(-c2ccc(Cl)cc2)c2cnccc12 ; OCCC1CCN(Cc2cn(-c3ccc(Cl)cc3)c3cnccc23)CC1 ; CN1CCN(Cc2cn(-c3ccc(Cl)cc3)c3cnccc23)CC1 ; OCC1CCN(Cc2cn(-c3ccc(Cl)cc3)c3cnccc23)CC1]

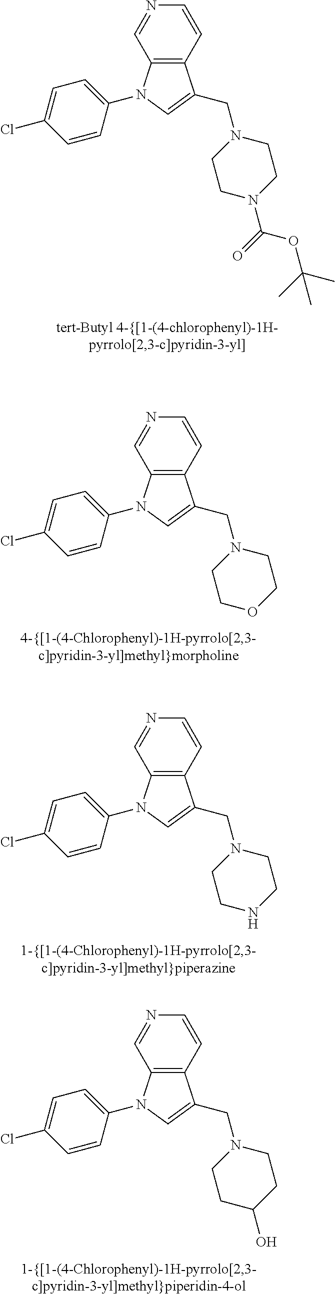 [SMILES: CC(C)(C)OC(=O)N1CCN(Cc2cn(-c3ccc(Cl)cc3)c3cnccc23)CC1 ; Clc1ccc(-n2cc(CN3CCOCC3)c3ccncc32)cc1 ; Clc1ccc(-n2cc(CN3CCNCC3)c3ccncc32)cc1 ; OC1CCN(Cc2cn(-c3ccc(Cl)cc3)c3cnccc23)CC1]

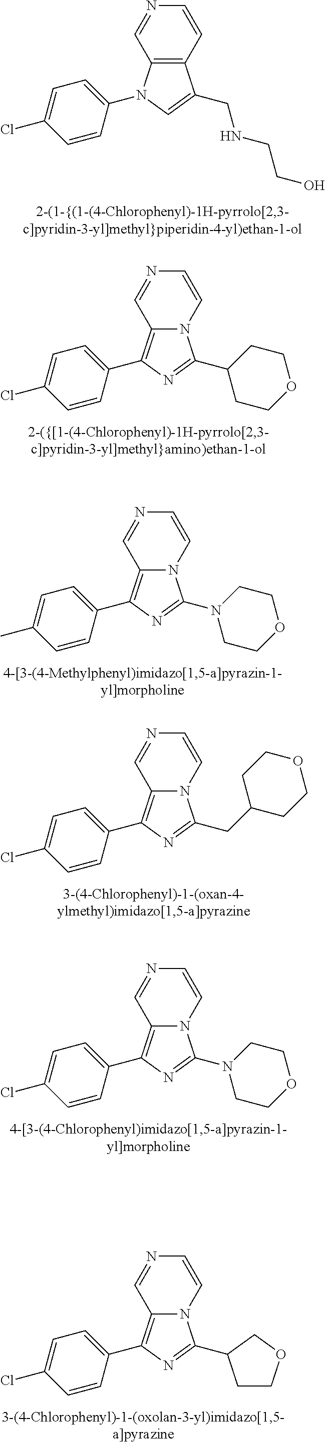 [SMILES: OCCNCc1cn(-c2ccc(Cl)cc2)c2cnccc12 ; Clc1ccc(-c2nc(C3CCOCC3)n3ccncc23)cc1 ; Cc1ccc(-c2nc(N3CCOCC3)n3ccncc23)cc1 ; Clc1ccc(-c2nc(CC3CCOCC3)n3ccncc23)cc1 ; Clc1ccc(-c2nc(N3CCOCC3)n3ccncc23)cc1 ; Clc1ccc(-c2nc(C3CCOC3)n3ccncc23)cc1]

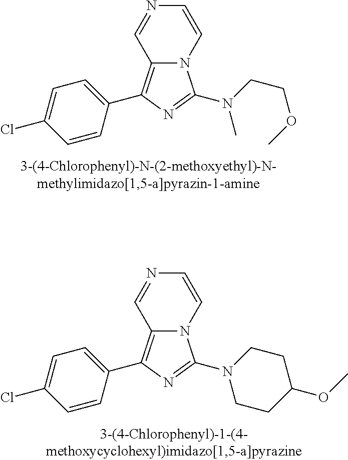 [SMILES: COCCN(C)c1nc(-c2ccc(Cl)cc2)c2cnccn12 ; COC1CCN(c2nc(-c3ccc(Cl)cc3)c3cnccn23)CC1]

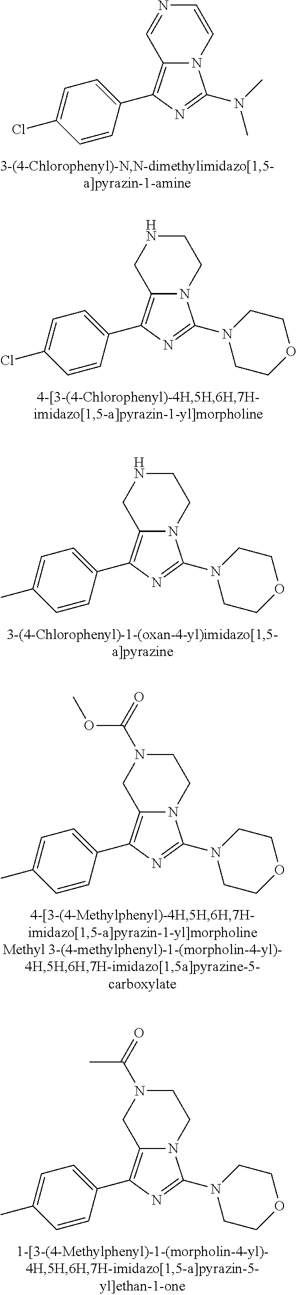

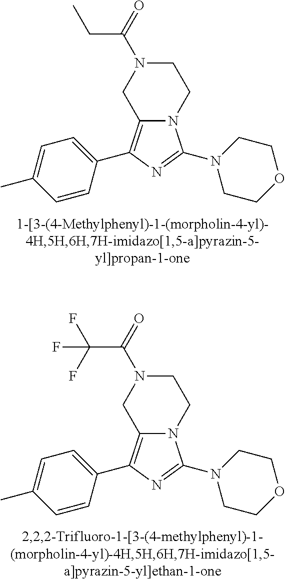 [SMILES: CCC(=O)N1CCn2c(N3CCOCC3)nc(-c3ccc(C)cc3)c2C1 ; Cc1ccc(-c2nc(N3CCOCC3)n3c2CN(C(=O)C(F)(F)F)CC3)cc1]

Further specific Examples of VAP-1 inhibitor compounds suitable for use in the present invention are provided below. Any pharmaceutically acceptable salt form of the Examples is suitable for use in the present invention. Specific examples of inhibitors of VAP-1 include:

the substituted 3-haloallylamine inhibitors specifically disclosed as Examples in WO 2013/163675, in particular compounds 1-39 in Table 1 of that document;

the IMIDAZO[4,5-C]PYRIDINE AND PYRROLO[2,3-C]PYRIDINE DERIVATIVES specifically disclosed as Examples in WO 2014/140592, namely:

### 3-[3-(4-Chlorophenyl)-3H-imidazo[4,5-c]pyridin-2-yl]pyridine

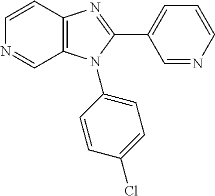 [SMILES: Clc1ccc(-n2c(-c3cccnc3)nc3ccncc32)cc1]

| Structure | Name |
|---|---|
| 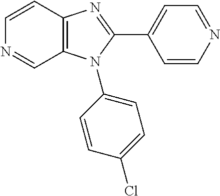 [SMILES: Clc1ccc(-n2c(-c3ccncc3)nc3ccncc32)cc1] | 4-[3-(4-Chlorophenyl)-3H- imidazo[4,5-c]pyridin-2-yl]pyridine |
| 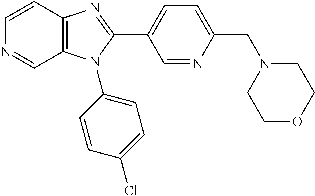 [SMILES: Clc1ccc(-n2c(-c3ccc(CN4CCOCC4)nc3)nc3ccncc32)cc1] | 4-({5-[3-(4-Chlorophenyl)-3H- imidazo[4,5-c]pyridin-2-yl]pyridin- 2-yl}methyl)morpholine |
| 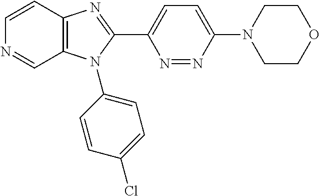 [SMILES: Clc1ccc(-n2c(-c3ccc(N4CCOCC4)nn3)nc3ccncc32)cc1] | 4-{6-[3-(4-Chlorophenyl)-3H- imidazo[4,5-c]pyridin-2- yl]pyridazin-3-yl}morpholine |
| 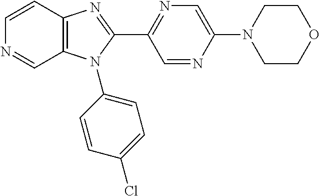 [SMILES: Clc1ccc(-n2c(-c3cnc(N4CCOCC4)cn3)nc3ccncc32)cc1] | 4-{5-[3-(4-Chlorophenyl)-3H- imidazo[4,5-c]pyridin-2-yl]pyrazin- 2-yl}morpholine |
| 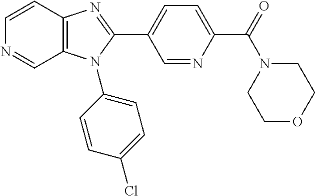 [SMILES: O=C(c1ccc(-c2nc3ccncc3n2-c2ccc(Cl)cc2)cn1)N1CCOCC1] | 4-({5-[3-(4-Chlorophenyl)-3H- imidazo[4,5-c]pyridin-2-yl]pyridin- 2-yl}carbonyl)morpholine |
| 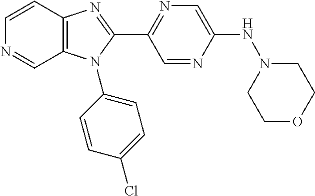 [SMILES: Clc1ccc(-n2c(-c3cnc(NN4CCOCC4)cn3)nc3ccncc32)cc1] | 5-[3-(4-Chlorophenyl)-3H- imidazo[4,5-c]pyridin-2-yl]-N- (oxan-4-yl)pyrazin-2-amine |
| 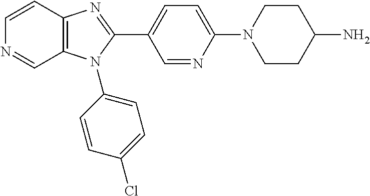 [SMILES: NC1CCN(c2ccc(-c3nc4ccncc4n3-c3ccc(Cl)cc3)cn2)CC1] | 1-{5-[3-(4-Chlorophenyl)-3H- imidazo[4,5-c]pyridin-2-yl]pyridin- 2-yl}piperidin-4-amine |
| 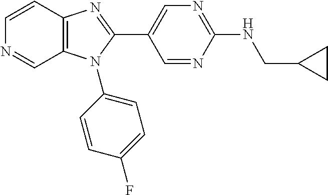 [SMILES: Fc1ccc(-n2c(-c3cnc(NCC4CC4)nc3)nc3ccncc32)cc1] | N-(Cyclopropylmethyl)-5-[3-(4- fluorophenyl)-3H-imidazo[4,5- c]pyridin-2-yl]pyrimidin-2-amine |
| 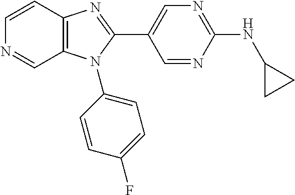 [SMILES: Fc1ccc(-n2c(-c3cnc(NC4CC4)nc3)nc3ccncc32)cc1] | N-Cyclopropyl-5-[3-(4- fluorophenyl)-3H-imidazo[4,5- c]pyridin-2-yl]pyrimidin-2-amine |
| 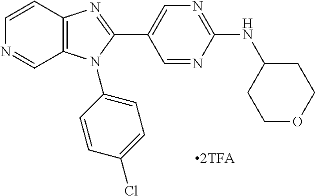 [SMILES: Clc1ccc(-n2c(-c3cnc(NC4CCOCC4)nc3)nc3ccncc32)cc1] | 5-[3-(4-Chlorophenyl)-3H- imidazo[4,5-c]pyridin-2-yl]-N- (oxan-4-yl)pyrimidin-2-amine; bis(trifluoroacetic acid) |
| 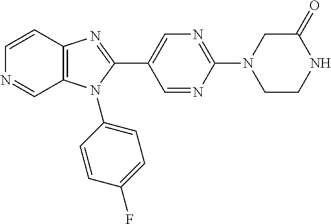 [SMILES: O=C1CN(c2ncc(-c3nc4ccncc4n3-c3ccc(F)cc3)cn2)CCN1] | 4-{5-[3-(4-Fluorophenyl)-3H- imidazo[4,5-c]pyridin-2- yl]pyrimidin-2-yl}piperazin-2-one |
| 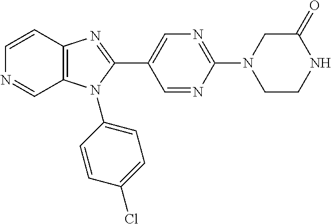 [SMILES: O=C1CN(c2ncc(-c3nc4ccncc4n3-c3ccc(Cl)cc3)cn2)CCN1] | 4-{5-[3-(4-Chlorophenyl)-3H- imidazo[4,5-c]pyridin-2- yl]pyrimidin-2-yl}piperazin-2-one |
| 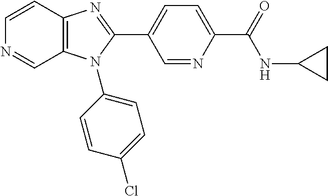 [SMILES: O=C(NC1CC1)c1ccc(-c2nc3ccncc3n2-c2ccc(Cl)cc2)cn1] | 5-[3-(4-Chlorophenyl)-3H- imidazo[4,5-c]pyridin-2-yl]-N- cyclopropylpyridine-2- carboxamide |
| 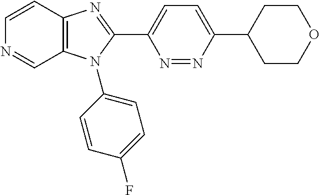 [SMILES: Fc1ccc(-n2c(-c3ccc(C4CCOCC4)nn3)nc3ccncc32)cc1] | 3-[3-(4-Fluorophenyl)-3H- imidazo[4,5-c]pyridin-2-yl]-6- (oxan-4-yl)pyridazine |
| 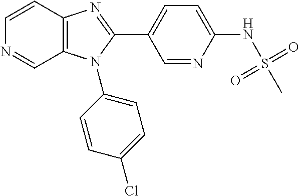 [SMILES: CS(=O)(=O)Nc1ccc(-c2nc3ccncc3n2-c2ccc(Cl)cc2)cn1] | N-{5-[3-(4-Chlorophenyl)-3H- imidazo[4,5-c]pyridin-2-yl]pyridin- 2-yl}methanesulfonamide |
| 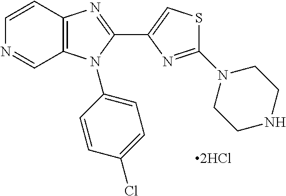 [SMILES: Clc1ccc(-n2c(-c3csc(N4CCNCC4)n3)nc3ccncc32)cc1] | 1-{4-[3-(4-Chlorophenyl)-3H- imidazo[4,5-c]pyridin-2-yl]-1,3- thiazol-2-yl}piperazine dihydrochloride |
| 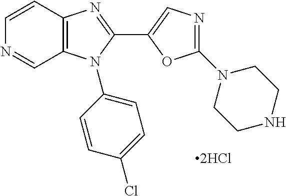 [SMILES: Clc1ccc(-n2c(-c3cnc(N4CCNCC4)o3)nc3ccncc32)cc1] | 1-{5-[3-(4-Chlorophenyl)-3H- imidazo[4,5-c]pyridin-2-yl]-1,3- oxazol-2-yl}piperazine dihydrochloride |
| 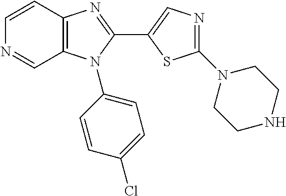 [SMILES: Clc1ccc(-n2c(-c3cnc(N4CCNCC4)s3)nc3ccncc32)cc1] | 1-{5-[3-(4-Chlorophenyl)-3H- imidazo[4,5-c]pyridin-2-yl]-1,3- thiazol-2-yl}piperazine |
| 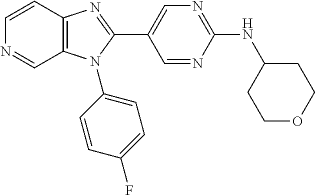 [SMILES: Fc1ccc(-n2c(-c3cnc(NC4CCOCC4)nc3)nc3ccncc32)cc1] | 5-[3-(4-Fluorophenyl)-3H- imidazo[4,5-c]pyridin-2-yl]-N- (oxan-4-yl)pyrimidin-2-amine |
| 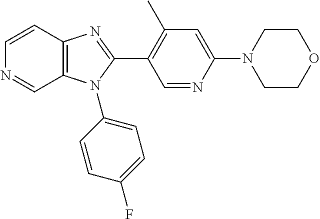 [SMILES: Cc1cc(N2CCOCC2)ncc1-c1nc2ccncc2n1-c1ccc(F)cc1] | 4-{5-[3-(4-Fluorophenyl)-3H- imidazo[4,5-c]pyridin-2-yl]-4- methylpyridin-2-yl}morpholine |
| 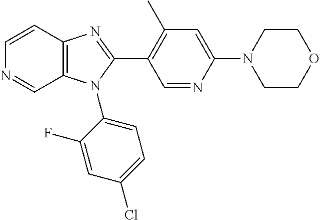 [SMILES: Cc1cc(N2CCOCC2)ncc1-c1nc2ccncc2n1-c1ccc(Cl)cc1F] | 4-{5-[3-(4-Chloro-2-fluorophenyl)- 3H-imidazo[4,5-c]pyridin-2-yl]-4- methylpyridin-2-yl}morpholine |
| 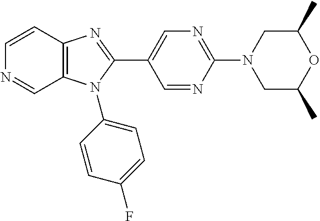 [SMILES: C[C@@H]1CN(c2ncc(-c3nc4ccncc4n3-c3ccc(F)cc3)cn2)C[C@H](C)O1] | (2R,6S)-4-{5-[3-(4-Fluorophenyl)- 3H-imidazo[4,5-c]pyridin-2- yl]pyrimidin-2-yl}-2,6- dimethylmorpholine |
| 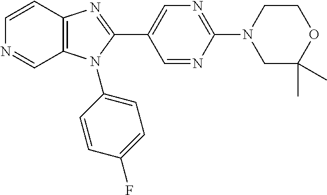 [SMILES: CC1(C)CN(c2ncc(-c3nc4ccncc4n3-c3ccc(F)cc3)cn2)CCO1] | 4-{5-[3-(4-Fluorophenyl)-3H- imidazo[4,5-c]pyridin-2- yl]pyrimidin-2-yl}-2,2- dimethylmorpholine |
| 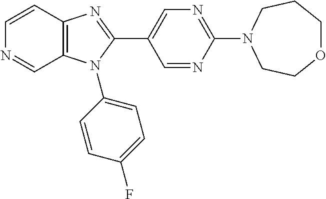 [SMILES: Fc1ccc(-n2c(-c3cnc(N4CCCOCC4)nc3)nc3ccncc32)cc1] | 4-{5-[3-(4-Fluorophenyl)-3H- imidazo[4,5-c]pyridin-2- yl]pyrimidin-2-yl}-1,4-oxazepane |
| 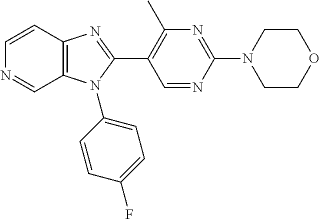 [SMILES: Cc1nc(N2CCOCC2)ncc1-c1nc2ccncc2n1-c1ccc(F)cc1] | 4-{5-[3-(4-Fluorophenyl)-3H- imidazo[4,5-c]pyridin-2-yl]-4- methylpyrimidin-2-yl}morpholine |
| 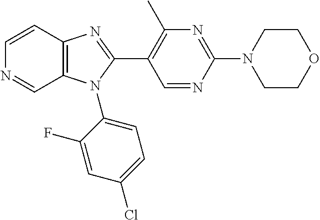 [SMILES: Cc1nc(N2CCOCC2)ncc1-c1nc2ccncc2n1-c1ccc(Cl)cc1F] | 4-{5-[3-(4-Chloro-2-fluorophenyl)- 3H-imidazo[4,5-c]pyridin-2-yl]-4- methylpyrimidin-2-yl}morpholine |
| 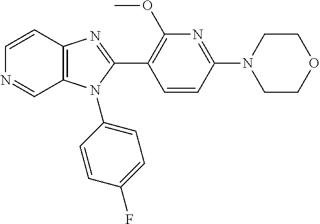 [SMILES: COc1nc(N2CCOCC2)ccc1-c1nc2ccncc2n1-c1ccc(F)cc1] | 4-{5-[3-(4-Fluorophenyl)-3H- imidazo[4,5-c]pyridin-2-yl]-6- methoxypyridin-2-yl}morpholine |
| 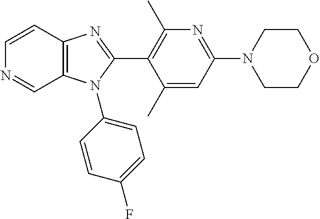 [SMILES: Cc1cc(N2CCOCC2)nc(C)c1-c1nc2ccncc2n1-c1ccc(F)cc1] | 4-{5-[3-(4-Fluorophenyl)-3H- imidazo[4,5-c]pyridin-2-yl]-4,6- dimethylpyridin-2-yl}morpholine |
| 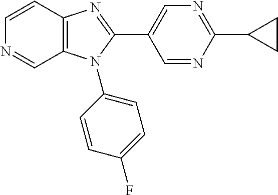 [SMILES: Fc1ccc(-n2c(-c3cnc(C4CC4)nc3)nc3ccncc32)cc1] | 2-Cyclopropyl-5-[3-(4- fluorophenyl)-3H-imidazo[4,5- c]pyridin-2-yl]pyrimidine |
| 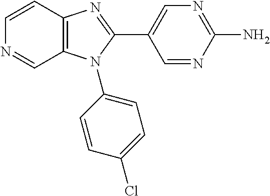 [SMILES: Nc1ncc(-c2nc3ccncc3n2-c2ccc(Cl)cc2)cn1] | 5-[3-(4-Chlorophenyl)-3H- imidazo[4,5-c]pyridin-2- yl]pyrimidin-2-amine |
| 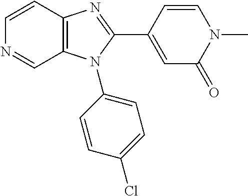 [SMILES: Cn1ccc(-c2nc3ccncc3n2-c2ccc(Cl)cc2)cc1=O] | 4-[3-(4-Chlorophenyl)-3H- imidazo[4,5-c]pyridin-2-yl]-1- methyl-1,2-dihydropyridin-2-one |
| 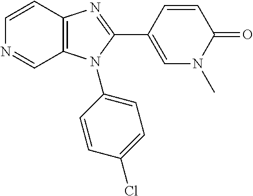 [SMILES: Cn1cc(-c2nc3ccncc3n2-c2ccc(Cl)cc2)ccc1=O] | 5-[3-(4-Chlorophenyl)-3H- imidazo[4,5-c]pyridin-2-yl]-1- methyl-1,2-dihydropyridin-2-one |
| 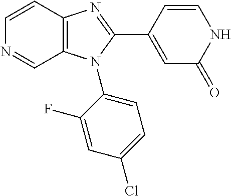 [SMILES: O=c1cc(-c2nc3ccncc3n2-c2ccc(Cl)cc2F)cc[nH]1] | 4-[3-(4-Chloro-2-fluorophenyl)- 3H-imidazo[4,5-c]pyridin-2-yl]- 1,2-dihydropyridin-2-one |
| 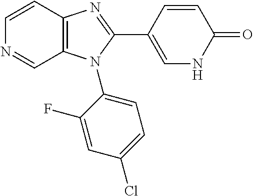 [SMILES: O=c1ccc(-c2nc3ccncc3n2-c2ccc(Cl)cc2F)c[nH]1] | 5-[3-(4-Chloro-2-fluorophenyl)- 3H-imidazo[4,5-c]pyridin-2-yl]- 1,2-dihydropyridin-2-one |
| 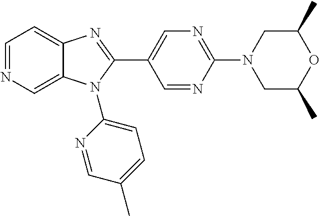 [SMILES: Cc1ccc(-n2c(-c3cnc(N4C[C@@H](C)O[C@@H](C)C4)nc3)nc3ccncc32)nc1] | (2R,6S)-2,6-Dimethyl-4-{5-[3-(5- methylpyridin-2-yl)-3H- imidazo[4,5-c]pyridin-2- yl]pyrimidin-2-yl}morpholine |
| 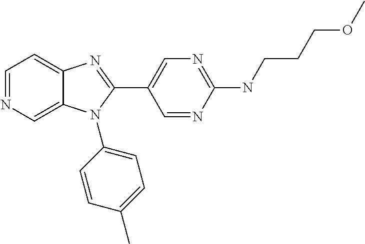 [SMILES: COCCCNc1ncc(-c2nc3ccncc3n2-c2ccc(C)cc2)cn1] | N-(3-Methoxypropyl)-5-[3-(4- methylphenyl)-3H-imidazo[4,5- c]pyridin-2-yl]pyrimidin-2-amine |
| 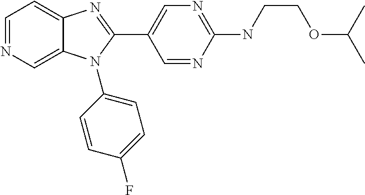 [SMILES: CC(C)OCCNc1ncc(-c2nc3ccncc3n2-c2ccc(F)cc2)cn1] | 5-[3-(4-Fluorophenyl)-3H- imidazo[4,5-c]pyridin-2-yl]-N-[2- (propan-2-yloxy)ethyl]pyrimidin-2- amine |
| 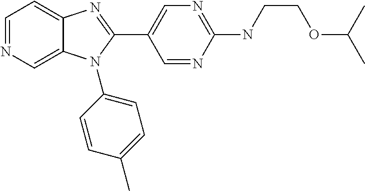 [SMILES: Cc1ccc(-n2c(-c3cnc(NCCOC(C)C)nc3)nc3ccncc32)cc1] | 5-[3-(4-Methylphenyl)-3H- imidazo[4,5-c]pyridin-2-yl]-N-[2- (propan-2-yloxy)ethyl]pyrimidin-2- amine |
| 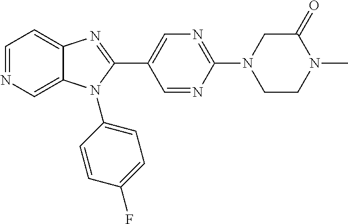 [SMILES: CN1CCN(c2ncc(-c3nc4ccncc4n3-c3ccc(F)cc3)cn2)CC1=O] | 4-{5-[3-(4-Fluorophenyl)-3H- imidazo[4,5-c]pyridin-2- yl]pyrimidin-2-yl}-1- methylpiperazin-2-one |
| 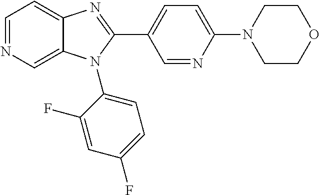 [SMILES: Fc1ccc(-n2c(-c3ccc(N4CCOCC4)nc3)nc3ccncc32)c(F)c1] | 4-{5-[3-(2,4-Difluorophenyl)-3H- imidazo[4,5-c]pyridin-2-yl]pyridin- 2-yl}morpholine |
| 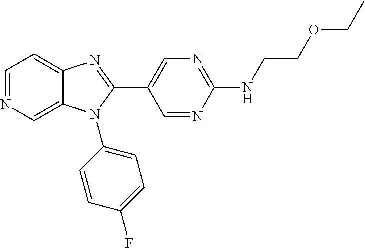 [SMILES: CCOCCNc1ncc(-c2nc3ccncc3n2-c2ccc(F)cc2)cn1] | N-(2-Ethoxyethyl)-5-[3-(4- fluorophenyl)-3H-imidazo[4,5- c]pyridin-2-yl]pyrimidin-2-amine |
| 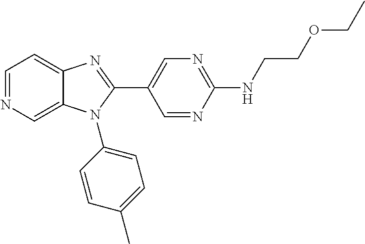 [SMILES: CCOCCNc1ncc(-c2nc3ccncc3n2-c2ccc(C)cc2)cn1] | N-(2-Ethoxyethyl)-5-[3-(4- methylphenyl)-3H-imidazo[4,5- c]pyridin-2-yl]pyrimidin-2-amine |

### 5-[3-(4-Chlorophenyl)-3H-imidazo[4,5-c]pyridin-2-yl]-1H-imidazole

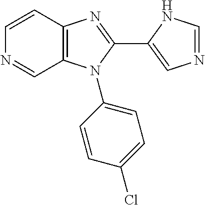 [SMILES: Clc1ccc(-n2c(-c3cnc[nH]3)nc3ccncc32)cc1]

| Structure | Name |
|---|---|
| 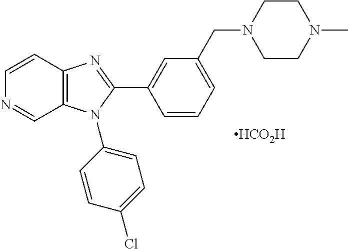 [SMILES: CN1CCN(Cc2cccc(-c3nc4ccncc4n3-c3ccc(Cl)cc3)c2)CC1 ; "·HCO2H"] | 1-({3-[3-(4-Chlorophenyl)-3H- imidazo[4,5-c]pyridin-2- yl]phenyl}methyl)-4- methylpiperazine; formic acid |
| 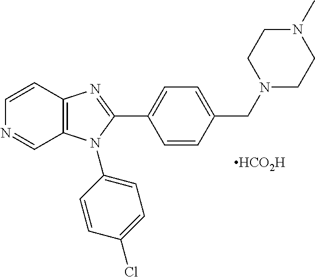 [SMILES: CN1CCN(Cc2ccc(-c3nc4ccncc4n3-c3ccc(Cl)cc3)cc2)CC1 ; "·HCO2H"] | 1-({4-[3-(4-Chlorophenyl)-3H- imidazo[4,5-c]pyridin-2- yl]phenyl}methyl)-4- methylpiperazine; formic acid |
| 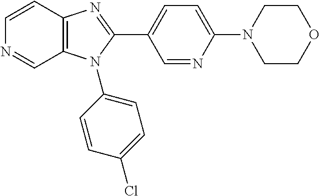 [SMILES: Clc1ccc(-n2c(-c3ccc(N4CCOCC4)nc3)nc3ccncc32)cc1] | 4-{5-[3-(4-Chlorophenyl)-3H- imidazo[4,5-c]pyridin-2-yl]pyridin- 2-yl}morpholine |
| 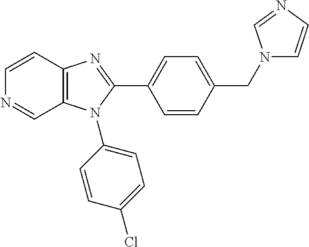 [SMILES: Clc1ccc(-n2c(-c3ccc(Cn4ccnc4)cc3)nc3ccncc32)cc1] | 1-({4-[3-(4-Chlorophenyl)-3H- imidazo[4,5-c]pyridin-2- yl]phenyl}methyl)-1H-imidazole |
| 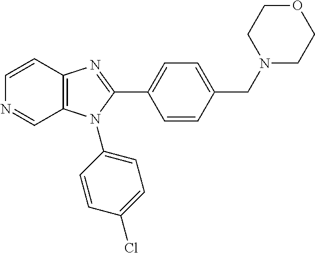 [SMILES: Clc1ccc(-n2c(-c3ccc(CN4CCOCC4)cc3)nc3ccncc32)cc1] | 4-({4-[3-(4-Chlorophenyl)-3H- imidazo[4,5-c]pyridin-2- yl]phenyl}methyl)morpholine |
| 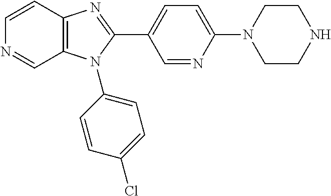 [SMILES: Clc1ccc(-n2c(-c3ccc(N4CCNCC4)nc3)nc3ccncc32)cc1] | 1-{5-[3-(4-Chlorophenyl)-3H- imidazo[4,5-c]pyridin-2-yl]pyridin- 2-yl}piperazine |
| 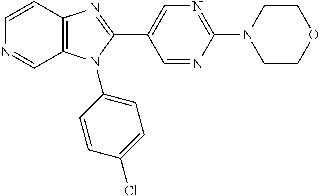 [SMILES: Clc1ccc(-n2c(-c3cnc(N4CCOCC4)nc3)nc3ccncc32)cc1] | 4-{5-[3-(4-Chlorophenyl)-3H- imidazo[4,5-c]pyridin-2- yl]pyrimidin-2-yl}morpholine |
| 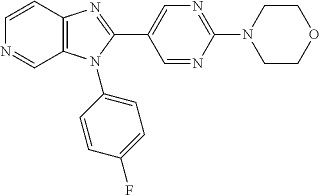 [SMILES: Fc1ccc(-n2c(-c3cnc(N4CCOCC4)nc3)nc3ccncc32)cc1] | 4-{5-[3-(4-Fluorophenyl)-3H- imidazo[4,5-c]pyridin-2- yl]pyrimidin-2-yl}morpholine |
| 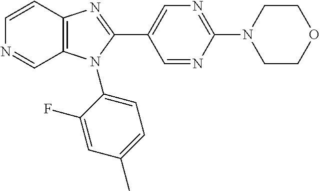 [SMILES: Cc1ccc(-n2c(-c3cnc(N4CCOCC4)nc3)nc3ccncc32)c(F)c1] | 4-{5-[3-(2-Fluoro-4- methylphenyl)-3H-imidazo[4,5- c]pyridin-2-yl]pyrimidin-2- yl}morpholine |
| 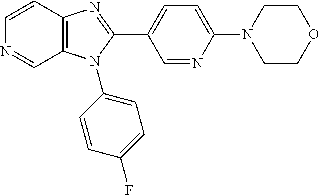 [SMILES: Fc1ccc(-n2c(-c3ccc(N4CCOCC4)nc3)nc3ccncc32)cc1] | 4-{5-[3-(4-Fluorophenyl)-3H- imidazo[4,5-c]pyridin-2-yl]pyridin- 2-yl}morpholine |
| 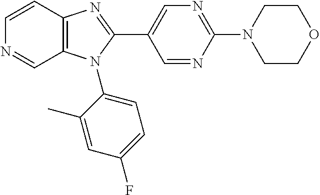 [SMILES: Cc1cc(F)ccc1-n1c(-c2cnc(N3CCOCC3)nc2)nc2ccncc21] | 4-{5-[3-(4-Fluoro-2- methylphenyl)-3H-imidazo[4,5- c]pyridin-2-yl]pyrimidin-2- yl}morpholine |
| 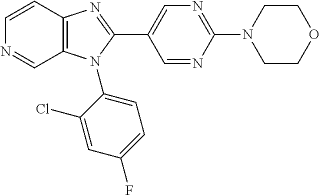 [SMILES: Fc1ccc(-n2c(-c3cnc(N4CCOCC4)nc3)nc3ccncc32)c(Cl)c1] | 4-{5-[3-(2-Chloro-4-fluorophenyl)- 3H-imidazo[4,5-c]pyridin-2- yl]pyrimidin-2-yl}morpholine |
| 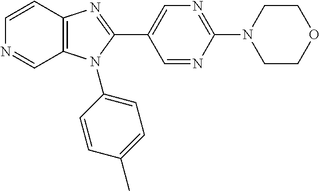 [SMILES: Cc1ccc(-n2c(-c3cnc(N4CCOCC4)nc3)nc3ccncc32)cc1] | 4-{5-[3-(4-Methylphenyl)-3H- imidazo[4,5-c]pyridin-2- yl]pyrimidin-2-yl}morpholine |
| 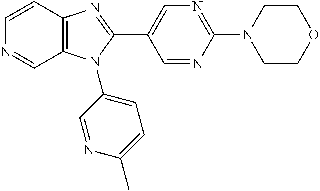 [SMILES: Cc1ccc(-n2c(-c3cnc(N4CCOCC4)nc3)nc3ccncc32)cn1] | 4-{5-[3-(6-Methylpyridin-3-yl)-3H- imidazo[4,5-c]pyridin-2- yl]pyrimidin-2-yl}morpholine |
| 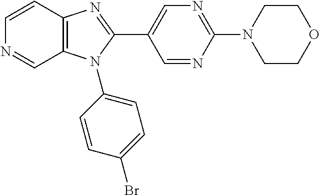 [SMILES: Brc1ccc(-n2c(-c3cnc(N4CCOCC4)nc3)nc3ccncc32)cc1] | 4-{5-[3-(4-Bromophenyl)-3H- imidazo[4,5-c]pyridin-2- yl]pyrimidin-2-yl}morpholine |
| 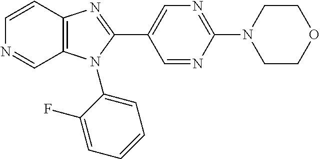 [SMILES: Fc1ccccc1-n1c(-c2cnc(N3CCOCC3)nc2)nc2ccncc21] | 4-{5-[3-(2-Fluorophenyl)-3H- imidazo[4,5-c]pyridin-2- yl]pyrimidin-2-yl}morpholine |
| 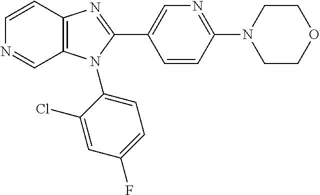 [SMILES: Fc1ccc(-n2c(-c3ccc(N4CCOCC4)nc3)nc3ccncc32)c(Cl)c1] | 4-{5-[3-(2-Chloro-4-fluorophenyl)- 3H-imidazo[4,5-c]pyridin-2- yl]pyridin-2-yl}morpholine |
| 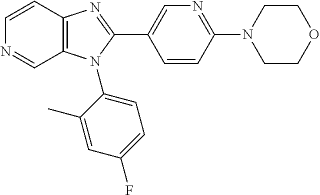 [SMILES: Cc1cc(F)ccc1-n1c(-c2ccc(N3CCOCC3)nc2)nc2ccncc21] | 4-{5-[3-(4-Fluoro-2- methylphenyl)-3H-imidazo[4,5- c]pyridin-2-yl]pyridin-2- yl}morpholine |
| 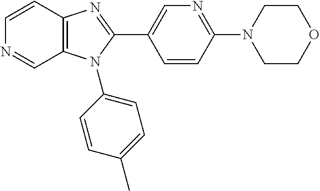 [SMILES: Cc1ccc(-n2c(-c3ccc(N4CCOCC4)nc3)nc3ccncc32)cc1] | 4-{5-[3-(4-Methylphenyl)-3H- imidazo[4,5-c]pyridin-2-yl]pyridin- 2-yl}morpholine |
| 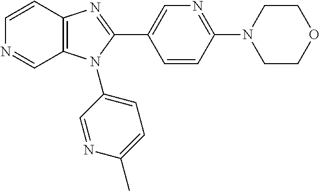 [SMILES: Cc1ccc(-n2c(-c3ccc(N4CCOCC4)nc3)nc3ccncc32)cn1] | 4-{5-[3-(6-Methylpyridin-3-yl)-3H- imidazo[4,5-c]pyridin-2-yl]pyridin- 2-yl}morpholine |
| 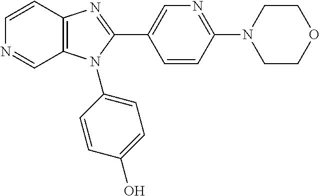 [SMILES: Oc1ccc(-n2c(-c3ccc(N4CCOCC4)nc3)nc3ccncc32)cc1] | 4-{2-[6-(Morpholin-4-yl)pyridin-3- yl]-3H-imidazo[4,5-c]pyridin-3- yl}phenol |
| 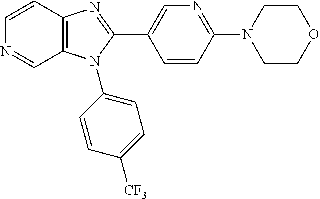 [SMILES: FC(F)(F)c1ccc(-n2c(-c3ccc(N4CCOCC4)nc3)nc3ccncc32)cc1] | 4-(5-{3-[4- (Trifluoromethyl)phenyl]-3H- imidazo[4,5-c]pyridin-2-yl}pyridin- 2-yl)morpholine |
| 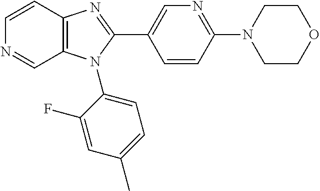 [SMILES: Cc1ccc(-n2c(-c3ccc(N4CCOCC4)nc3)nc3ccncc32)c(F)c1] | 4-{5-[3-(2-Fluoro-4- methylphenyl)-3H-imidazo[4,5- c]pyridin-2-yl]pyridin-2- yl}morpholine |
| 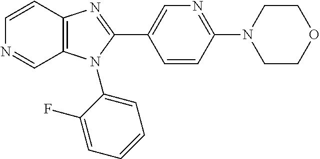 [SMILES: Fc1ccccc1-n1c(-c2ccc(N3CCOCC3)nc2)nc2ccncc21] | 4-{5-[3-(2-Fluorophenyl)-3H- imidazo[4,5-c]pyridin-2-yl]pyridin- 2-yl}morpholine |
| 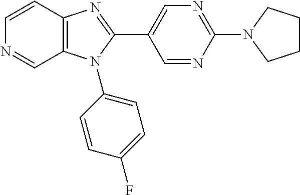 [SMILES: Fc1ccc(-n2c(-c3cnc(N4CCCC4)nc3)nc3ccncc32)cc1] | 5-[3-(4-Fluorophenyl)-3H- imidazo[4,5-c]pyridin-2-yl]-2- (pyrrolidin-1-yl)pyrimidine |
| 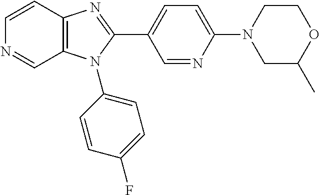 [SMILES: CC1CN(c2ccc(-c3nc4ccncc4n3-c3ccc(F)cc3)cn2)CCO1] | 4-{5-[3-(4-Fluorophenyl)-3H- imidazo[4,5-c]pyridin-2-yl]pyridin- 2-yl}-2-methylmorpholine |
| 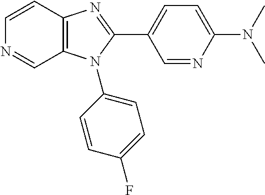 [SMILES: CN(C)c1ccc(-c2nc3ccncc3n2-c2ccc(F)cc2)cn1] | 5-[3-(4-Fluorophenyl)-3H- imidazo[4,5-c]pyridin-2-yl]-N,N- dimethylpyridin-2-amine |
| 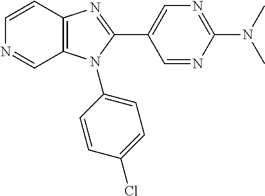 [SMILES: CN(C)c1ncc(-c2nc3ccncc3n2-c2ccc(Cl)cc2)cn1] | 5-[3-(4-Chlorophenyl)-3H- imidazo[4,5-c]pyridin-2-yl]-N,N- dimethylpyrimidin-2-amine |

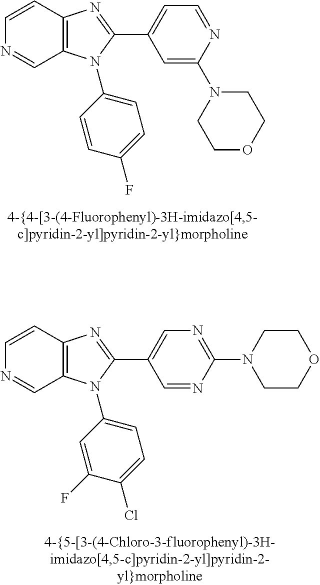 [SMILES: Fc1ccc(-n2c(-c3ccnc(N4CCOCC4)c3)nc3ccncc32)cc1 ; Fc1cc(-n2c(-c3cnc(N4CCOCC4)nc3)nc3ccncc32)ccc1Cl]

| Structure | Name |
|---|---|
| 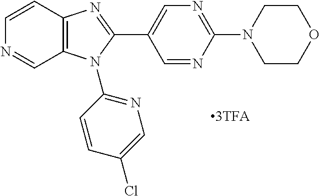 [SMILES: Clc1ccc(-n2c(-c3cnc(N4CCOCC4)nc3)nc3ccncc32)nc1] | 4-{5-[3-(5-Chloropyridin-2-yl)-3H- imidazo[4,5-c]pyridin-2- yl]pyrimidin-2-yl}morpholine; tris(trifluoroacetic acid) |
| 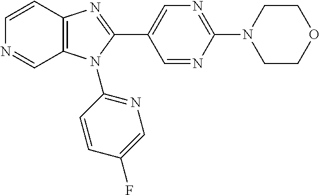 [SMILES: Fc1ccc(-n2c(-c3cnc(N4CCOCC4)nc3)nc3ccncc32)nc1] | 4-{5-[3-(5-Fluoropyridin-2-yl)-3H- imidazo[4,5-c]pyridin-2- yl]pyrimidin-2-yl}morpholine |
| 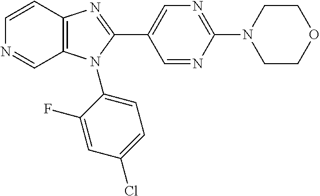 [SMILES: Fc1cc(Cl)ccc1-n1c(-c2cnc(N3CCOCC3)nc2)nc2ccncc21] | 4-{5-[3-(4-Chloro-2-fluorophenyl)- 3H-imidazo[4,5-c]pyridin-2- yl]pyrimidin-2-yl}morpholine |
| 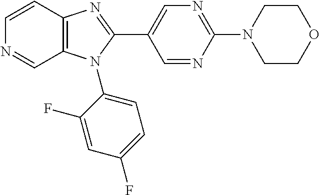 [SMILES: Fc1ccc(-n2c(-c3cnc(N4CCOCC4)nc3)nc3ccncc32)c(F)c1] | 4-{5-[3-(2,4-Difluorophenyl)-3H- imidazo[4,5-c]pyridin-2- yl]pyrimidin-2-yl}morpholine |
| 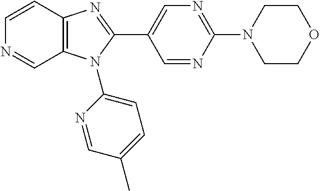 [SMILES: Cc1ccc(-n2c(-c3cnc(N4CCOCC4)nc3)nc3ccncc32)nc1] | 4-{5-[3-(5-Methylpyridin-2-yl)-3H- imidazo[4,5-c]pyridin-2- yl]pyrimidin-2-yl}morpholine |
| 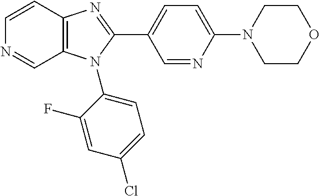 [SMILES: Fc1cc(Cl)ccc1-n1c(-c2ccc(N3CCOCC3)nc2)nc2ccncc21] | 4-{5-[3-(4-Chloro-2-fluorophenyl)- 3H-imidazo[4,5-c]pyridin-2- yl]pyridin-2-yl}morpholine |
| 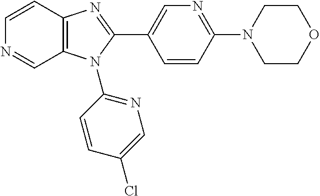 [SMILES: Clc1ccc(-n2c(-c3ccc(N4CCOCC4)nc3)nc3ccncc32)nc1] | 4-{5-[3-(5-Chloropyridin-2-yl)-3H- imidazo[4,5-c]pyridin-2-yl]pyridin- 2-yl}morpholine |
| 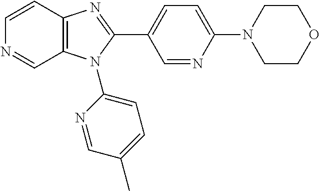 [SMILES: Cc1ccc(-n2c(-c3ccc(N4CCOCC4)nc3)nc3ccncc32)nc1] | 4-{5-[3-(5-Methylpyridin-2-yl)-3H- imidazo[4,5-c]pyridin-2-yl]pyridin- 2-yl}morpholine |
| 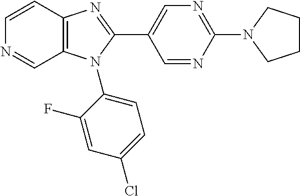 [SMILES: Fc1cc(Cl)ccc1-n1c(-c2cnc(N3CCCC3)nc2)nc2ccncc21] | 5-[3-(4-Chloro-2-fluorophenyl)- 3H-imidazo[4,5-c]pyridin-2-yl]-2- (pyrrolidin-1-yl)pyrimidine |
| 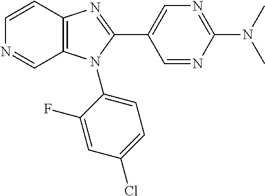 [SMILES: CN(C)c1ncc(-c2nc3ccncc3n2-c2ccc(Cl)cc2F)cn1] | 5-[3-(4-Chloro-2-fluorophenyl)- 3H-imidazo[4,5-c]pyridin-2-yl]- N,N-dimethylpyrimidin-2-amine |

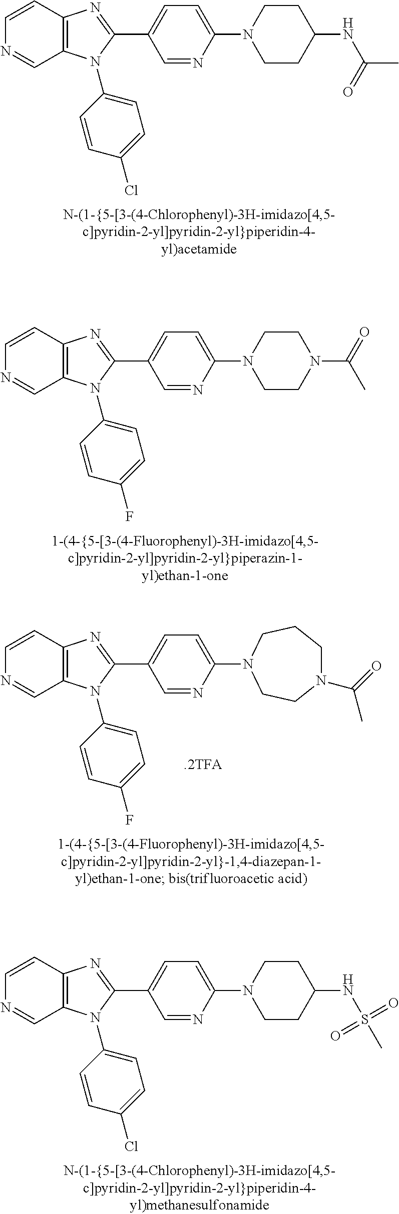 [SMILES: CC(=O)NC1CCN(c2ccc(-c3nc4ccncc4n3-c3ccc(Cl)cc3)cn2)CC1 ; CC(=O)N1CCN(c2ccc(-c3nc4ccncc4n3-c3ccc(F)cc3)cn2)CC1 ; CC(=O)N1CCCN(c2ccc(-c3nc4ccncc4n3-c3ccc(F)cc3)cn2)CC1 ; CS(=O)(=O)NC1CCN(c2ccc(-c3nc4ccncc4n3-c3ccc(Cl)cc3)cn2)CC1]

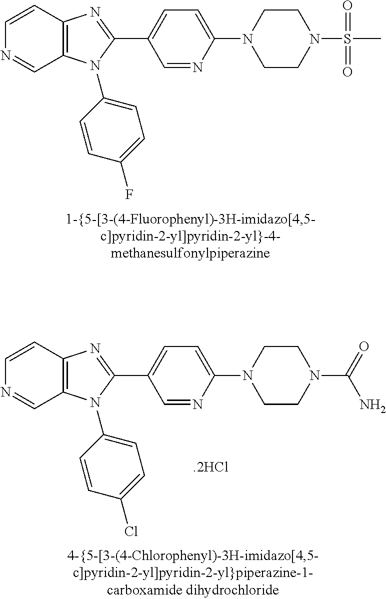 [SMILES: CS(=O)(=O)N1CCN(c2ccc(-c3nc4ccncc4n3-c3ccc(F)cc3)cn2)CC1 ; NC(=O)N1CCN(c2ccc(-c3nc4ccncc4n3-c3ccc(Cl)cc3)cn2)CC1]

| Structure | Name |
|---|---|
| 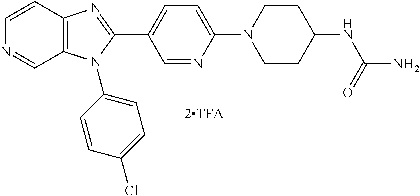 [SMILES: NC(=O)NC1CCN(c2ccc(-c3nc4ccncc4n3-c3ccc(Cl)cc3)cn2)CC1] | (1-{5-[3-(4-Chlorophenyl)- 3H-imidazo[4,5-c]pyridin- 2-yl]pyridin-2-yl}piperidin- 4-yl)urea; bis(trifluoroacetic acid) |
| 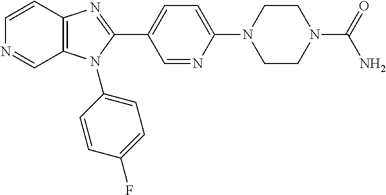 [SMILES: NC(=O)N1CCN(c2ccc(-c3nc4ccncc4n3-c3ccc(F)cc3)cn2)CC1] | 4-{5-[3-(4-Fluorophenyl)- 3H-imidazo[4,5-c]pyridin- 2-yl]pyridin-2- yl}piperazine-1- carboxamide |
| 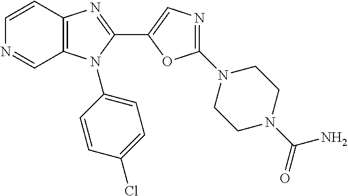 [SMILES: NC(=O)N1CCN(c2ncc(-c3nc4ccncc4n3-c3ccc(Cl)cc3)o2)CC1] | 4-{5-[3-(4-Chlorophenyl)- 3H-imidazo[4,5-c]pyridin- 2-yl]-1,3-oxazol-2- yl}piperazine-1- carboxamide |
| 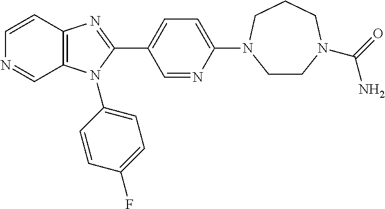 [SMILES: NC(=O)N1CCCN(c2ccc(-c3nc4ccncc4n3-c3ccc(F)cc3)cn2)CC1] | 4-{5-[3-(4-Fluorophenyl)- 3H-imidazo[4,5-c]pyridin- 2-yl]pyridin-2-yl}-1,4- diazepane-1-carboxamide |

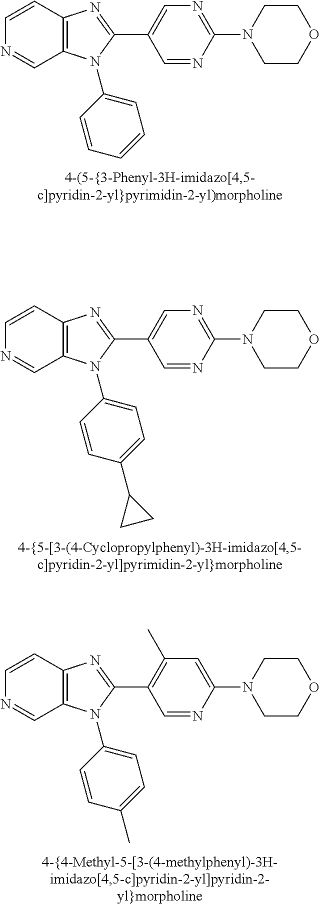 [SMILES: c1ccc(-n2c(-c3cnc(N4CCOCC4)nc3)nc3ccncc32)cc1 ; c1cc2nc(-c3cnc(N4CCOCC4)nc3)n(-c3ccc(C4CC4)cc3)c2cn1 ; Cc1ccc(-n2c(-c3cnc(N4CCOCC4)cc3C)nc3ccncc32)cc1]

| Structure | Name |
|---|---|
| 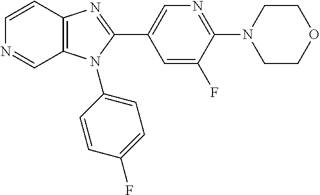 [SMILES: Fc1ccc(-n2c(-c3cnc(N4CCOCC4)c(F)c3)nc3ccncc32)cc1] | 4-{3-Fluoro-5-[3-(4-fluorophenyl)- 3H-imidazo[4,5-c]pyridin-2- yl]pyridin-2-yl}morpholine |
| 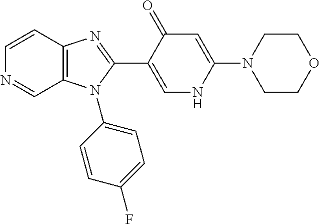 [SMILES: O=c1cc(N2CCOCC2)[nH]cc1-c1nc2ccncc2n1-c1ccc(F)cc1] | 5-[3-(4-Fluorophenyl)-3H- imidazo[4,5-c]pyridin-2-yl]-2- (morpholin-4-yl)-1,4- dihydropyridin-4-one |
| 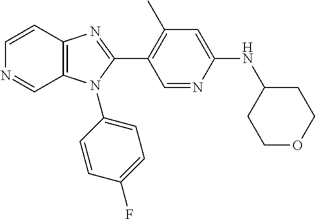 [SMILES: Cc1cc(NC2CCOCC2)ncc1-c1nc2ccncc2n1-c1ccc(F)cc1] | 5-[3-(4-Fluorophenyl)-3H- imidazo[4,5-c]pyridin-2-yl]-4- methyl-N-(oxan-4-yl)pyridin-2- amine |
| 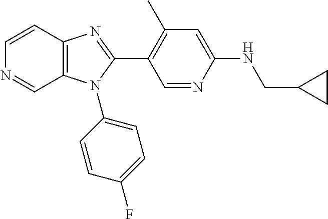 [SMILES: Cc1cc(NCC2CC2)ncc1-c1nc2ccncc2n1-c1ccc(F)cc1] | N-(Cyclopropylmethyl)-5-[3-(4- fluorophenyl)-3H-imidazo[4,5- c]pyridin-2-yl]-4-methylpyridin-2- amine |
| 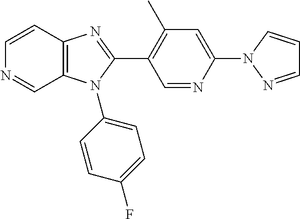 [SMILES: Cc1cc(-n2cccn2)ncc1-c1nc2ccncc2n1-c1ccc(F)cc1] | 5-[3-(4-Fluorophenyl)-3H- imidazo[4,5-c]pyridin-2-yl]-4- methyl-2-(1H-pyrazol-1- yl)pyridine |
| 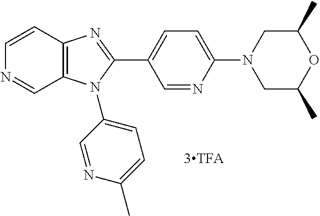 [SMILES: Cc1ccc(-n2c(-c3ccc(N4C[C@@H](C)O[C@@H](C)C4)nc3)nc3ccncc32)cn1] | (2R,6S)-2,6-Dimethyl-4-{5-[3-(6- methylpyridin-3-yl)-3H- imidazo[4,5-c]pyridin-2-yl]pyridin- 2-yl}morpholine; tris(trifluoroacetic acid) |
| 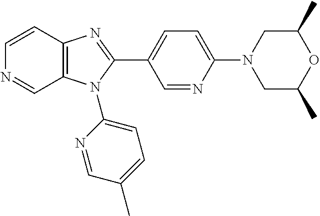 [SMILES: Cc1ccc(-n2c(-c3ccc(N4C[C@@H](C)O[C@@H](C)C4)nc3)nc3ccncc32)nc1] | (2R,6S)-2,6-Dimethyl-4-{5-[3-(5- methylpyridin-2-yl)-3H- imidazo[4,5-c]pyridin-2-yl]pyridin- 2-yl}morpholine |
| 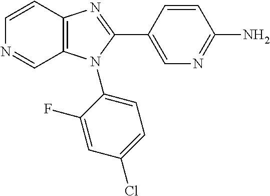 [SMILES: Nc1ccc(-c2nc3ccncc3n2-c2ccc(Cl)cc2F)cn1] | 5-[3-(4-Chloro-2-fluorophenyl)- 3H-imidazo[4,5-c]pyridin-2- yl]pyridin-2-amine |
| 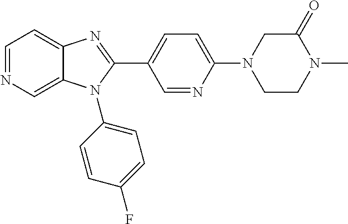 [SMILES: CN1CCN(c2ccc(-c3nc4ccncc4n3-c3ccc(F)cc3)cn2)CC1=O] | 4-{5-[3-(4-Fluorophenyl)-3H- imidazo[4,5-c]pyridin-2-yl]pyridin- 2-yl}-1-methylpiperazin-2-one |
| 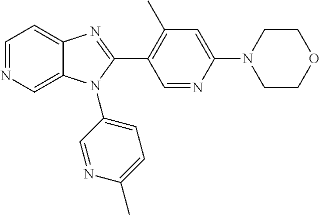 [SMILES: Cc1ccc(-n2c(-c3cnc(N4CCOCC4)cc3C)nc3ccncc32)cn1] | 4-{4-Methyl-5-[3-(6-methylpyridin- 3-yl)-3H-imidazo[4,5-c]pyridin-2- yl]pyridin-2-yl}morpholine |

### 4-{5-[3-(4-Fluorophenyl)-3H-imidazo[4,5-c]pyridin-2-yl]-6-methylpyridin-2-yl}morpholine

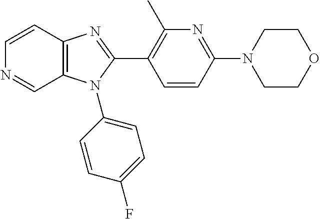 [SMILES: Cc1nc(N2CCOCC2)ccc1-c1nc2ccncc2n1-c1ccc(F)cc1]

| Structure | Name |
|---|---|
| 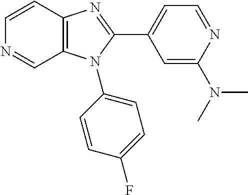 [SMILES: CN(C)c1cc(-c2nc3ccncc3n2-c2ccc(F)cc2)ccn1] | 4-[3-(4-Fluorophenyl)-3H- imidazo[4,5-c]pyridin-2- yl]-N,N-dimethylpyridin- 2-amine |
| 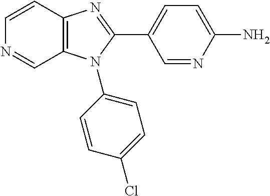 [SMILES: Nc1ccc(-c2nc3ccncc3n2-c2ccc(Cl)cc2)cn1] | 5-[3-(4-Chlorophenyl)-3H- imidazo[4,5-c]pyridin-2- yl]pyridin-2-amine |
| 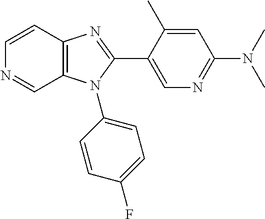 [SMILES: Cc1cc(N(C)C)ncc1-c1nc2ccncc2n1-c1ccc(F)cc1] | 5-[3-(4-Fluorophenyl)-3H- imidazo[4,5-c]pyridin-2- yl]-N,N,4- trimethylpyridin-2- amine |

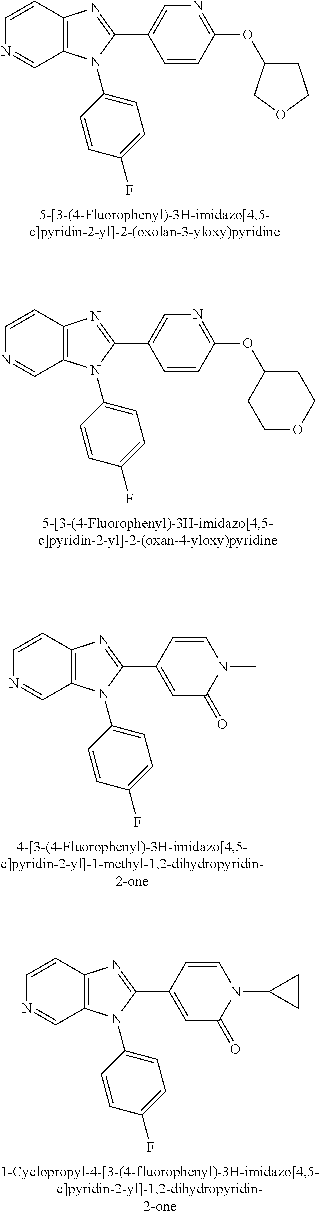 [SMILES: Fc1ccc(-n2c(-c3ccc(OC4CCOC4)nc3)nc3ccncc32)cc1 ; Fc1ccc(-n2c(-c3ccc(OC4CCOCC4)nc3)nc3ccncc32)cc1 ; Cn1ccc(-c2nc3ccncc3n2-c2ccc(F)cc2)cc1=O ; O=c1cc(-c2nc3ccncc3n2-c2ccc(F)cc2)ccn1C1CC1]

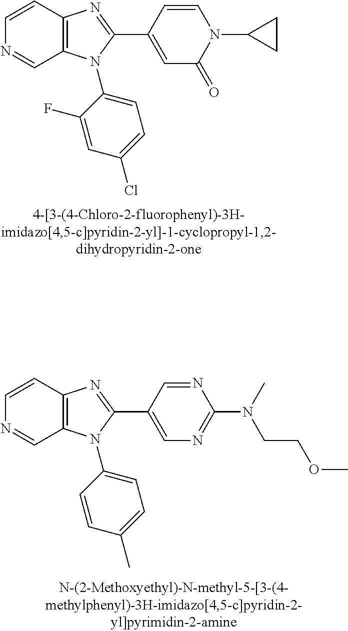 [SMILES: O=c1cc(-c2nc3ccncc3n2-c2ccc(Cl)cc2F)ccn1C1CC1 ; COCCN(C)c1ncc(-c2nc3ccncc3n2-c2ccc(C)cc2)cn1]

| Structure | Name |
|---|---|
| 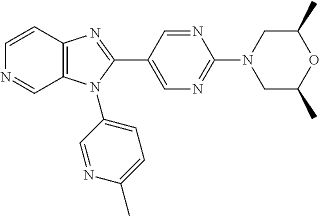 [SMILES: Cc1ccc(-n2c(-c3cnc(N4C[C@@H](C)O[C@@H](C)C4)nc3)nc3ccncc32)cn1] | (2R,6S)-2,6-Dimethyl-4-{5-[3-(6- methylpyridin-3-yl)-3H- imidazo[4,5-c]pyridin-2- yl]pyrimidin-2-yl}morpholine |
| 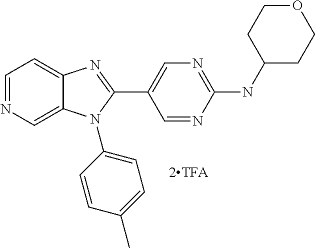 [SMILES: Cc1ccc(-n2c(-c3cnc(NC4CCOCC4)nc3)nc3ccncc32)cc1] | 5-[3-(4-Methylphenyl)-3H- imidazo[4,5-c]pyridin-2-yl]-N- (oxan-4-yl)pyrimidin-2-amine; bis(trifluoroacetic acid) |

### 4-[1-(4-Chlorophenyl)-1H-pyrrolo[2,3-c]pyridin-2-yl]pyridine

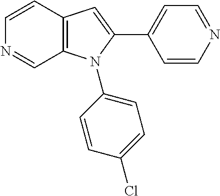 [SMILES: Clc1ccc(-n2c(-c3ccncc3)cc3ccncc32)cc1]

| Structure | Name |
|---|---|
| 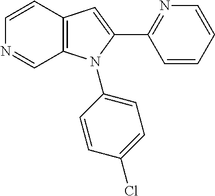 [SMILES: Clc1ccc(-n2c(-c3ccccn3)cc3ccncc32)cc1] | 2-[1-(4-Chloro- phenyl)-1H- pyrrolo[2,3-c] pyridin-2-yl] pyridine |
| 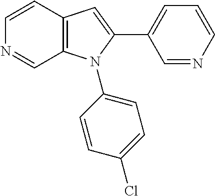 [SMILES: Clc1ccc(-n2c(-c3cccnc3)cc3ccncc32)cc1] | 3-[1-(4-Chloro- phenyl)-1H- pyrrolo[2,3-c] pyridin-2-yl] pyridine |
| 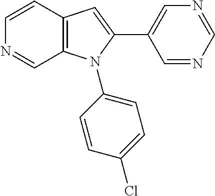 [SMILES: Clc1ccc(-n2c(-c3cncnc3)cc3ccncc32)cc1] | 5-[1-(4-Chloro- phenyl)-1H- pyrrolo[2,3-c] pyridin-2- yl]pyrimidine |
| 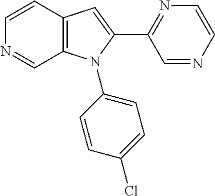 [SMILES: Clc1ccc(-n2c(-c3cnccn3)cc3ccncc32)cc1] | 2-[1-(4-Chloro- phenyl)-1H- pyrrolo[2,3-c] pyridin-2-yl] pyrazine |
| 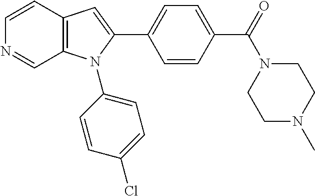 [SMILES: CN1CCN(C(=O)c2ccc(-c3cc4ccncc4n3-c3ccc(Cl)cc3)cc2)CC1] | 1-({4-[1-(4-Chloro- phenyl)-1H- pyrrolo[2,3-c] pyridin-2- yl]phenyl} carbonyl)-4- methyl- piperazine |
| 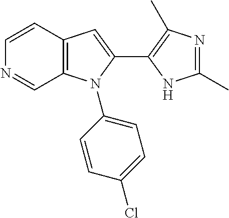 [SMILES: Cc1nc(C)c(-c2cc3ccncc3n2-c2ccc(Cl)cc2)[nH]1] | 5-[1-(4-Chloro- phenyl)-1H- pyrrolo[2,3-c] pyridin-2-yl]-2,4- dimethyl-1H- imidazole |
| 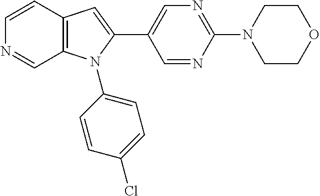 [SMILES: Clc1ccc(-n2c(-c3cnc(N4CCOCC4)nc3)cc3ccncc32)cc1] | 4-{5-[1-(4-Chloro- phenyl)-1H- pyrrolo[2,3-c] pyridin-2- yl]pyrimidin-2- yl}morpholine |
| 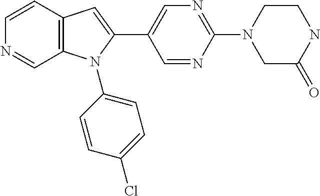 [SMILES: O=C1CN(c2ncc(-c3cc4ccncc4n3-c3ccc(Cl)cc3)cn2)CCN1] | 4-{5-[1-(4- Chlorophenyl)- 1H-pyrrolo [2,3-c]pyridin-2- yl]pyrimidin-2- yl}piperazin-2- one |
| 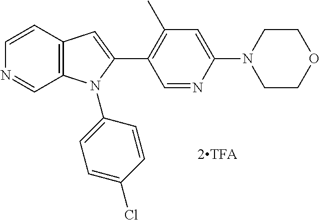 [SMILES: Cc1cc(N2CCOCC2)ncc1-c1cc2ccncc2n1-c1ccc(Cl)cc1] | 4-{5-[1-(4- Chlorophenyl)- 1H-pyrrolo [2,3-c]pyridin- 2-yl]-4- methylpyridin- 2-yl}morpholine; bis(trifluoroacetic acid) |
| 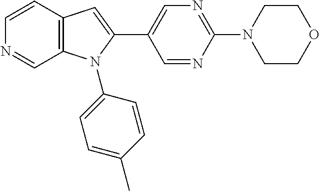 [SMILES: Cc1ccc(-n2c(-c3cnc(N4CCOCC4)nc3)cc3ccncc32)cc1] | 4-{5-[1-(4- Methylphenyl)- 1H-pyrrolo [2,3-c]pyridin-2- yl]pyrimidin-2- yl}morpholine |
| 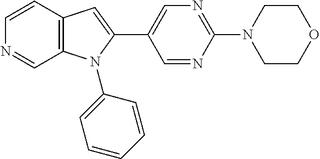 [SMILES: c1ccc(-n2c(-c3cnc(N4CCOCC4)nc3)cc3ccncc32)cc1] | 4-(5-{1-Phenyl- 1H-pyrrolo[2,3- c]pyridin-2-yl} pyrimidin-2- yl)morpholine |
| 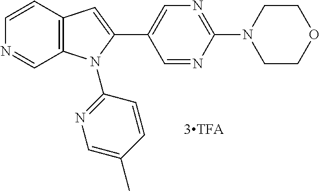 [SMILES: Cc1ccc(-n2c(-c3cnc(N4CCOCC4)nc3)cc3ccncc32)nc1] | 4-{5-[1-(5- Methylpyridin- 2-yl)-1H- pyrrolo[2,3-c] pyridin-2- yl]pyrimidin- 2-yl}morpholine; tris(trifluoroacetic acid) |
| 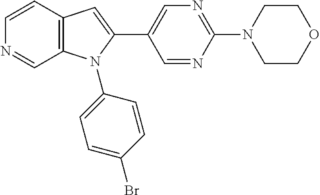 [SMILES: Brc1ccc(-n2c(-c3cnc(N4CCOCC4)nc3)cc3ccncc32)cc1] | 4-{5-[1-(4- Bromophenyl)- 1H-pyrrolo [2,3-c]pyridin- 2-yl]pyrimidin- 2-yl}morpholine |
| 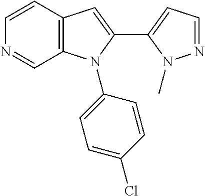 [SMILES: Cn1nccc1-c1cc2ccncc2n1-c1ccc(Cl)cc1] | 5-[1-(4-Chloro- phenyl)-1H- pyrrolo[2,3-c] pyridin-2-yl]-1- methyl-1H- pyrazole |
| 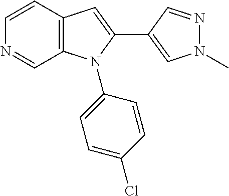 [SMILES: Cn1cc(-c2cc3ccncc3n2-c2ccc(Cl)cc2)cn1] | 4-[1-(4-Chloro- phenyl)-1H- pyrrolo[2,3-c] pyridin-2-yl]-1- methyl-1H- pyrazole |
| 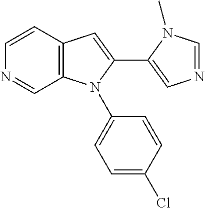 [SMILES: Cn1cncc1-c1cc2ccncc2n1-c1ccc(Cl)cc1] | 5-[1-(4-Chloro- phenyl)-1H- pyrrolo[2,3-c] pyridin-2-yl]-1- methyl-1H- imidazole |
| 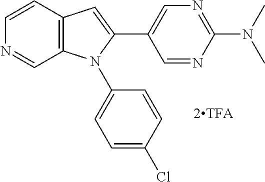 [SMILES: CN(C)c1ncc(-c2cc3ccncc3n2-c2ccc(Cl)cc2)cn1] | 5-[1-(4-Chloro- phenyl)-1H- pyrrolo[2,3-c] pyridin-2-yl]- N,N-dimethyl- pyrimidin-2- amine; bis(trifluoroacetic acid) |
| 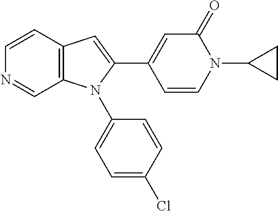 [SMILES: O=c1cc(-c2cc3ccncc3n2-c2ccc(Cl)cc2)ccn1C1CC1] | 4-[1-(4-Chloro- phenyl)-1H- pyrrolo[2,3-c] pyridin-2-yl]-1- cyclopropyl-1,2- dihydropyridin- 2-one |
| 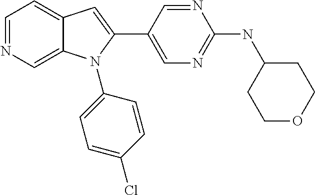 [SMILES: Clc1ccc(-n2c(-c3cnc(NC4CCOCC4)nc3)cc3ccncc32)cc1] | 5-[1-(4-Chloro- phenyl)-1H- pyrrolo[2,3-c] pyridin-2-yl]-N- (oxan-4-yl) pyrimidin-2- amine |
| 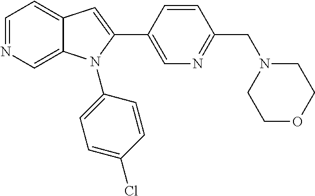 [SMILES: Clc1ccc(-n2c(-c3ccc(CN4CCOCC4)nc3)cc3ccncc32)cc1] | 4-({5-[1-(4- Chlorophenyl)- 1H-pyrrolo [2,3-c]pyridin- 2-yl]pyridin- 2-yl}methyl) morpholine |
| 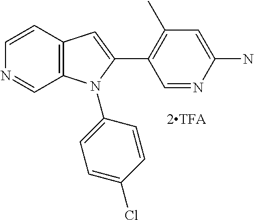 [SMILES: Cc1cc(N)ncc1-c1cc2ccncc2n1-c1ccc(Cl)cc1] | 5-[1-(4-Chloro- phenyl)-1H- pyrrolo[2,3-c] pyridin-2-yl]-4- methylpyridin- 2-amine; bis(trifluoroacetic acid) |
| 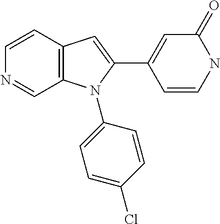 [SMILES: O=c1cc(-c2cc3ccncc3n2-c2ccc(Cl)cc2)cc[nH]1] | 4-[1-(4-Chloro- phenyl)-1H- pyrrolo[2,3-c] pyridin-2-yl]- 1,2-dihydro- pyridin-2-one |
| 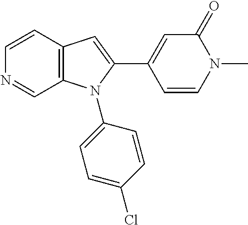 [SMILES: Cn1ccc(-c2cc3ccncc3n2-c2ccc(Cl)cc2)cc1=O] | 4-[1-(4-Chloro- phenyl)-1H- pyrrolo[2,3-c] pyridin-2-yl]-1- methyl-1,2- dihydropyridin- 2-one |
| 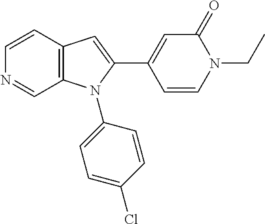 [SMILES: CCn1ccc(-c2cc3ccncc3n2-c2ccc(Cl)cc2)cc1=O] | 4-[1-(4-Chloro- phenyl)-1H- pyrrolo[2,3-c] pyridin-2-yl]- 1-ethyl-1,2- dihydropyridin- 2-one |
| 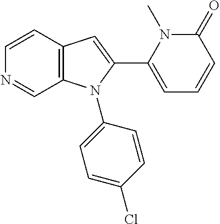 [SMILES: Cn1c(-c2cc3ccncc3n2-c2ccc(Cl)cc2)cccc1=O] | 6-[1-(4-Chloro- phenyl)-1H- pyrrolo[2,3-c] pyridin-2-yl]-1- methyl-1,2- dihydropyridin-2- one |
| 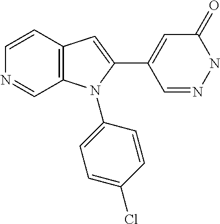 [SMILES: O=c1cc(-c2cc3ccncc3n2-c2ccc(Cl)cc2)cn[nH]1] | 5-[1-(4-Chloro- phenyl)-1H- pyrrolo[2,3-c] pyridin-2-yl]- 2,3-dihydro- pyridazin-3-one |
| 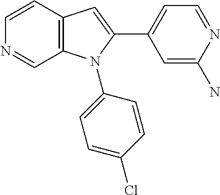 [SMILES: Nc1cc(-c2cc3ccncc3n2-c2ccc(Cl)cc2)ccn1] | 4-[1-(4-Chloro- phenyl)-1H- pyrrolo[2,3-c] pyridin-2-yl] pyridin-2-amine |
| 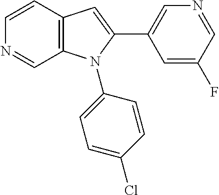 [SMILES: Fc1cncc(-c2cc3ccncc3n2-c2ccc(Cl)cc2)c1] | 3-[1-(4-Chloro- phenyl)-1H- pyrrolo[2,3-c] pyridin-2-yl]-5- fluoropyridine |
| 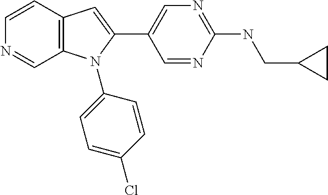 [SMILES: Clc1ccc(-n2c(-c3cnc(NCC4CC4)nc3)cc3ccncc32)cc1] | 5-[1-(4-Chloro- phenyl)-1H- pyrrolo[2,3-c] pyridin-2-yl]- N-(cyclopropyl- methyl) pyrimidin-2- amine |
| 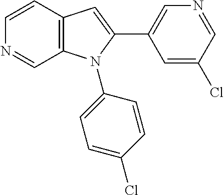 [SMILES: Clc1ccc(-n2c(-c3cncc(Cl)c3)cc3ccncc32)cc1] | 3-Chloro-5-[1- (4-chlorophenyl)- 1H-pyrrolo [2,3-c]pyridin-2- yl]pyridine |
| 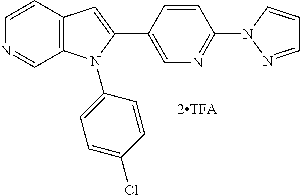 [SMILES: Clc1ccc(-n2c(-c3ccc(-n4cccn4)nc3)cc3ccncc32)cc1] | 5-[1-(4-Chloro- phenyl)-1H- pyrrolo[2,3-c] pyridin-2-yl]- 2-(1H- pyrazol-1-yl) pyridine; bis(trifluoroacetic acid) |
| 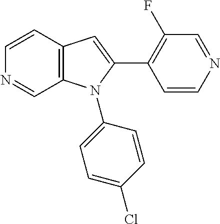 [SMILES: Fc1cnccc1-c1cc2ccncc2n1-c1ccc(Cl)cc1] | 4-[1-(4-Chloro- phenyl)-1H- pyrrolo[2,3-c] pyridin-2-yl]-3- fluoropyridine |
| 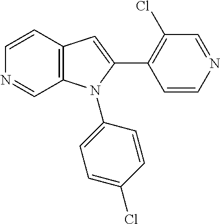 [SMILES: Clc1ccc(-n2c(-c3ccncc3Cl)cc3ccncc32)cc1] | 3-Chloro-4-[1- (4-chlorophenyl)- 1H-pyrrolo [2,3-c]pyridin-2- yl]pyridine |
| 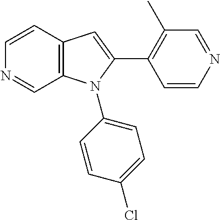 [SMILES: Cc1cnccc1-c1cc2ccncc2n1-c1ccc(Cl)cc1] | 4-[1-(4-Chloro- phenyl)-1H- pyrrolo[2,3-c] pyridin-2-yl]-3- methylpyridine |

### 1-Cyclopropyl-4-{1-phenyl-1H-pyrrolo[2,3-c]pyridin-2-yl}-1,2-dihydropyridin-2-one

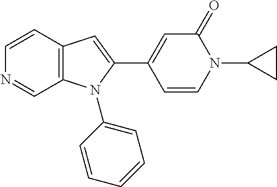 [SMILES: O=c1cc(-c2cc3ccncc3n2-c2ccccc2)ccn1C1CC1]

the inhibitors of SSAO activity specifically disclosed as Examples in WO2014/140591, namely:

### 4-[2-(6-Aminopyridin-3-yl)-1-(4-chlorophenyl)-1H-pyrrolo[2,3-c]pyridin-3-yl]piperidine-1-carboxamide

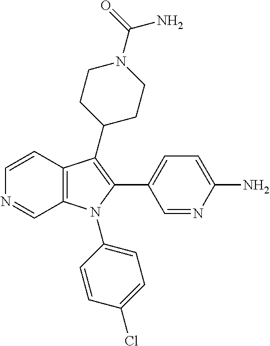 [SMILES: NC(=O)N1CCC(c2c(-c3ccc(N)nc3)n(-c3ccc(Cl)cc3)c3cnccc23)CC1]

| Structure | Name |
|---|---|
| 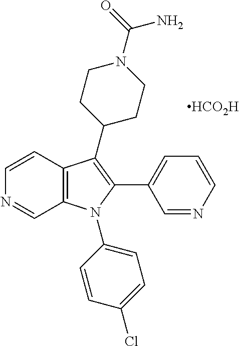 [SMILES: NC(=O)N1CCC(c2c(-c3cccnc3)n(-c3ccc(Cl)cc3)c3cnccc23)CC1 ; "·HCO2H"] | 4-[1-(4-Chloro- phenyl)-2- (pyridin-3-yl)- 1H-pyrrolo [2,3-c]pyridin- 3-yl]piperidine- 1-carboxamide; formic acid |
| 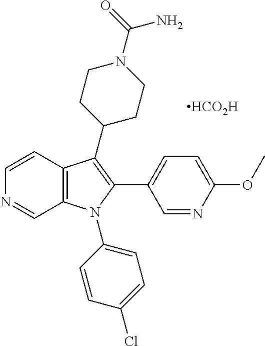 [SMILES: COc1ccc(-c2c(C3CCN(C(N)=O)CC3)c3ccncc3n2-c2ccc(Cl)cc2)cn1 ; "·HCO2H"] | 4-[1-(4-Chloro- phenyl)-2-(6- methoxy- pyridin-3-yl)- 1H-pyrrolo [2,3-c]pyridin- 3-yl]piperidine- 1-carboxamide; formic acid |
| 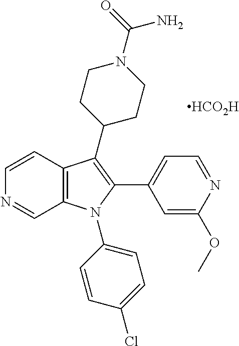 [SMILES: COc1cc(-c2c(C3CCN(C(N)=O)CC3)c3ccncc3n2-c2ccc(Cl)cc2)ccn1 ; "·HCO2H"] | 4-[1-(4-Chloro- phenyl)-2-(2- methoxy- pyridin-4-yl)- 1H-pyrrolo [2,3-c]pyridin- 3-yl]piperidine- 1-carboxamide; formic acid |
| 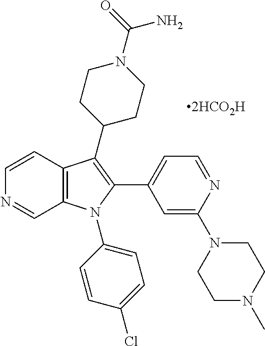 [SMILES: CN1CCN(c2cc(-c3c(C4CCN(C(N)=O)CC4)c4ccncc4n3-c3ccc(Cl)cc3)ccn2)CC1 ; "·2HCO2H"] | 4-[1-(4-Chloro- phenyl)-2-[2-(4- methyl- piperazin-1-yl) pyridin-4-1H- pyrrolo[2,3-c] pyridin-3-yl] piperidine-1- carboxamide; bis(formic acid) |
| 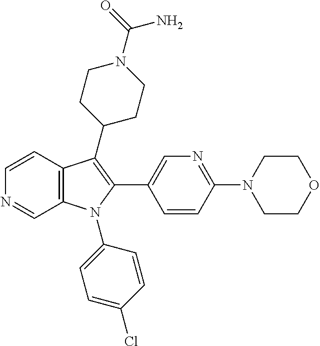 [SMILES: NC(=O)N1CCC(c2c(-c3ccc(N4CCOCC4)nc3)n(-c3ccc(Cl)cc3)c3cnccc23)CC1] | 4-[1-(4-Chloro- phenyl)-2-[6- (morpholin-4-yl) pyridin-3-yl]- 1H-pyrrolo [2,3-c]pyridin-3- yl]piperidine-1- carboxamide |
| 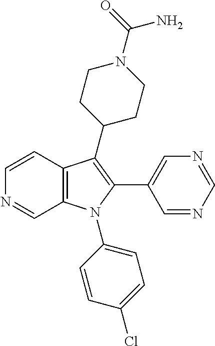 [SMILES: NC(=O)N1CCC(c2c(-c3cncnc3)n(-c3ccc(Cl)cc3)c3cnccc23)CC1] | 4-[1-(4-Chloro- phenyl)-2- (pyrimidin-5-yl)- 1H-pyrrolo[2,3- c]pyridin-3-yl] piperidine-1- carboxamide |
| 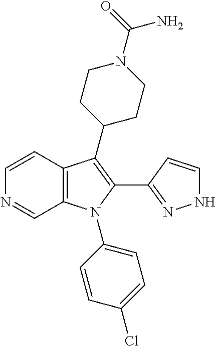 [SMILES: NC(=O)N1CCC(c2c(-c3cc[nH]n3)n(-c3ccc(Cl)cc3)c3cnccc23)CC1] | 4-[1-(4-Chloro- phenyl)-2-(1H- pyrazol-3-yl)- 1H-pyrrolo[2,3- c]pyridin-3-yl] piperidine-1- carboxamide |
| 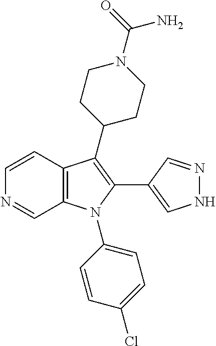 [SMILES: NC(=O)N1CCC(c2c(-c3cn[nH]c3)n(-c3ccc(Cl)cc3)c3cnccc23)CC1] | 4-[1-(4-Chloro- phenyl)-2-(1H- pyrazol-4-yl)- 1H-pyrrolo[2,3- c]pyridin-3-yl] piperidine-1- carboxamide |
| 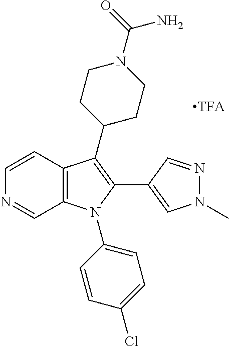 [SMILES: Cn1cc(-c2c(C3CCN(C(N)=O)CC3)c3ccncc3n2-c2ccc(Cl)cc2)cn1 ; "·TFA"] | 4-[1-(4-Chloro- phenyl)-2-(1- methyl-1H- pyrazol-4-yl)- 1H-pyrrolo [2,3-c]pyridin-3- yl]piperidine-1- carboxamide; trifluoroacetic acid |

### 4-[1-(4-Chlorophenyl)-2-(1-methyl-1H-pyrazol-5-yl)-1H-pyrrolo[2,3-c]pyridin-3-yl]piperidine-1-carboxamide

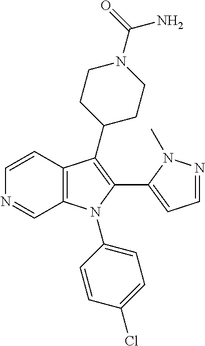 [SMILES: Cn1nccc1-c1c(C2CCN(C(N)=O)CC2)c2ccncc2n1-c1ccc(Cl)cc1]

| Structure | Name |
|---|---|
| 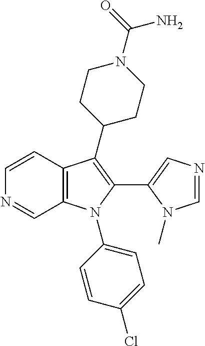 [SMILES: Cn1cncc1-c1c(C2CCN(C(N)=O)CC2)c2ccncc2n1-c1ccc(Cl)cc1] | 4-[1-(4-Chloro- phenyl)-2-(1- methyl-1H- imidazol-5-yl)- 1H-pyrrolo[2,3-c] pyridin-3-yl] piperidine-1- carboxamide |
| 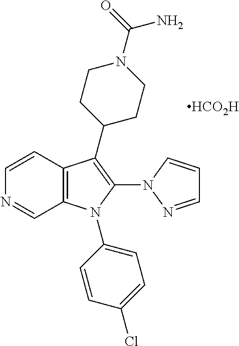 [SMILES: NC(=O)N1CCC(c2c(-n3cccn3)n(-c3ccc(Cl)cc3)c3cnccc23)CC1 ; "·HCO2H"] | 4-[1-(4-Chloro- phenyl)-2-(1H- pyrazol-1-yl)- 1H-pyrrolo[2,3- c]pyridin-3-yl] piperidine-1- carboxamide; formic acid |
| 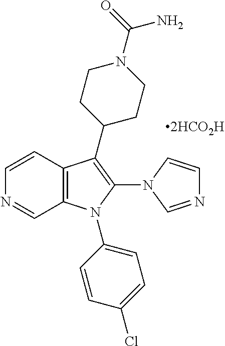 [SMILES: NC(=O)N1CCC(c2c(-n3ccnc3)n(-c3ccc(Cl)cc3)c3cnccc23)CC1 ; "·2HCO2H"] | 4-[1-(4-Chloro- phenyl)-2-(1H- imidazol-1-yl)- 1H-pyrrolo[2,3- c]pyridin-3-yl] piperidine-1- carboxamide; bis (formic acid) |
| 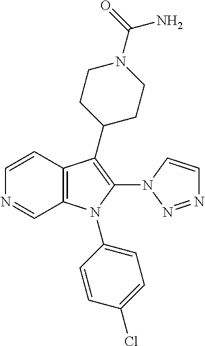 [SMILES: NC(=O)N1CCC(c2c(-n3ccnn3)n(-c3ccc(Cl)cc3)c3cnccc23)CC1] | 4-[1-(4-Chloro- phenyl)-2-(1H- 1,2,3-triazol-1- yl)-1H-pyrrolo [2,3-c]pyridin-3- yl]piperidine-1- carboxamide |
| 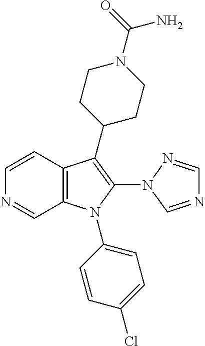 [SMILES: NC(=O)N1CCC(c2c(-n3cncn3)n(-c3ccc(Cl)cc3)c3cnccc23)CC1] | 4-[1-(4-Chloro- phenyl)-2-(1H- 1,2,4-triazol-1-yl)- 1H-pyrrolo[2,3- c]pyridin-3-yl] piperidine-1- carboxamide |

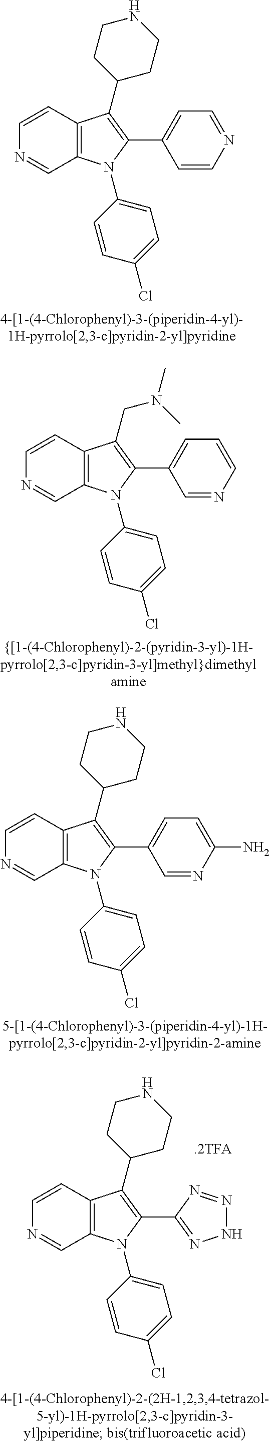 [SMILES: Clc1ccc(-n2c(-c3ccncc3)c(C3CCNCC3)c3ccncc32)cc1 ; CN(C)Cc1c(-c2cccnc2)n(-c2ccc(Cl)cc2)c2cnccc12 ; Nc1ccc(-c2c(C3CCNCC3)c3ccncc3n2-c2ccc(Cl)cc2)cn1 ; Clc1ccc(-n2c(-c3nn[nH]n3)c(C3CCNCC3)c3ccncc32)cc1]

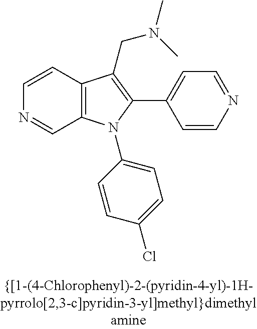 [SMILES: CN(C)Cc1c(-c2ccncc2)n(-c2ccc(Cl)cc2)c2cnccc12]

In an embodiment, the VAP-1 inhibitor suitable for use in the present invention is selected from the group consisiting of:

Procarbazine

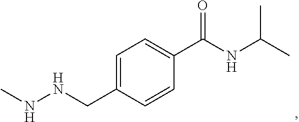 [SMILES: CNNCc1ccc(C(=O)NC(C)C)cc1]

Isocarboxazid

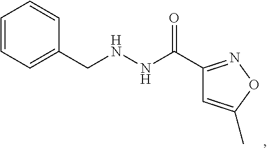 [SMILES: Cc1cc(C(=O)NNCc2ccccc2)no1]

Guanabenx

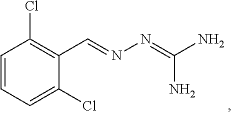 [SMILES: NC(N)=N/N=C/c1c(Cl)cccc1Cl]

Carbidopa

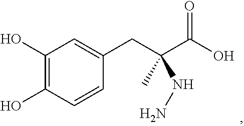 [SMILES: C[C@@](Cc1ccc(O)c(O)c1)(NN)C(=O)O]

and

Benserazide

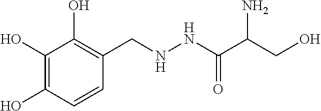 [SMILES: NC(CO)C(=O)NNCc1ccc(O)c(O)c1O]

and pharmaceutically acceptable salts thereof.

Racemic Carbidopa is useful in the present invention. Preferably the Carbidopa for use in the invention is the (R) enantiomer or the (S) enantiomer. Racemic Benserazide is preferred for use in the present invention. In an embodiment the Benserazide for use in the present invention is the (R) enantiomer or the (S) enantiomer.

In a particular embodiment of the invention, there is provided benserazide, or a pharmaceutically acceptable salt thereof, for use in the treatment of muscular dystrophy, particularly Duchenne muscular dystrophy, in a human subject.

Compositions

For clinical use, the VAP-1 inhibitor compounds of the invention are formulated into pharmaceutical formulations for various modes of administration. It will be appreciated that compounds may be administered together with a physiologically acceptable carrier, excipient, or diluent. The pharmaceutical compositions of the invention may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Formulations may conveniently be presented in unit dosage form, e.g., tablets and sustained release capsules, and in liposomes, and may be prepared by any method known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight; administered singly or multiply in doses, e.g. from about 0.01 mg to about 25 mg each. Such a dosage may be given orally or parenterally. Multiple doses may be administered over a period of time, such as at least a week, a month, several months, a year, or several years, or throughout the course of the condition. The frequency of dosage may be at least once per month, once per week, or once per day.

Combined Preparations

The components of a combined preparation of the invention may be for simultaneous, separate, or sequential use.

The term “combined preparation” as used herein refers to a “kit of parts” in the sense that the combination components (a) and (b) can be dosed independently or by use of different fixed combinations with distinguished amounts of the combination components (a) and (b). The components can be administered simultaneously or one after the other. If the components are administered one after the other, preferably the time interval between administration is chosen such that the effect on the treated disorder or disease in the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a patient sub-population to be treated, or the needs of the single patient, which can be due, for example, to the particular disease, age, sex, or body weight of the patients.

Preferably, there is at least one beneficial effect, for example an enhancing of the effect of the VAP-1 inhibitor, or a mutual enhancing of the effect of the combination components (a) and (b), for example a more than additive effect, additional advantageous effects, fewer side effects, less toxicity, or a combined therapeutic effect compared with a non-effective dosage of one or both of the combination components (a) and (b), and very preferably a synergism of the combination components (a) and (b).

The VAP-1 inhibitor and the steroid may be administered sequentially to the subject, i.e. the VAP-1 inhibitor may be administered before, with, or after the steroid.

The VAP-1 inhibitor and the steroid may be administered to the subject within 96 hours, 72 hours, 48 hours, 24 hours, or 12 hours, of each other.

Alternatively, the VAP-1 inhibitor and the steroid may be co-administered to the subject, for example as a composition comprising the VAP-1 inhibitor and the steroid, or by simultaneous administration of separate doses of the VAP-1 inhibitor and the steroid.

According to some embodiments, a plurality of doses of the VAP-1 inhibitor, and/or a plurality of doses of the steroid, is administered to the subject.

According to some embodiments, a dose of the VAP-1 inhibitor is administered before, with, or after each administration of two or more doses of the steroid.

For example, a dose of VAP-1 inhibitor may be administered within 96 hours, 72 hours, 48 hours, 24 hours, or 12 hours, of each administration of two or more doses of the steroid.

The choice of appropriate dosages of the components used in combination therapy according to the present invention can be determined and optimized by the skilled person, for example, by observation of the patient, including the patient's overall health, and the response to the combination therapy. Optimization, for example, may be necessary if it is determined that a patient is not exhibiting the desired therapeutic effect or conversely, if the patient is experiencing undesirable or adverse side effects that are too many in number or are of a troublesome severity.

The doses of the components used in combination therapy according to the invention should be chosen to provide a therapeutically effective amount of the components in combination. An “effective amount” of the combination therapy is an amount that results in a reduction of at least one pathological parameter associated with muscular dystrophy. For example, in some embodiments, an effective amount of the combination therapy is an amount that is effective to achieve a reduction of at least about 10%, 20%, 30%, 40%, 50%, 60%, 70%, 80%, or 90%, in the parameter, compared to the expected reduction in the parameter associated with the muscular dystrophy without the combination therapy. For example, the parameter may be: muscle pathology; muscle degeneration; muscle necrosis; inflammation of muscle; infiltration of inflammatory cells into muscle, especially inflammatory myeloid cells such as monocytes, macrophages, neutrophils, or eosinophils; infiltration of lymphoid cells into muscle, especially T helper lymphocytes or double negative T cells; inflammatory cell activation in muscle; muscle fibrosis; or fibrotic cell activation in muscle.

According to the invention, combination treatment may be employed to increase the therapeutic effect of the VAP-1 inhibitor or steroid, compared with the effect of the VAP-1 inhibitor or steroid as a monotherapy, or to decrease the doses of the individual components in the resulting combinations while preventing or further reducing the risk of unwanted or harmful side effects of the individual components.

Bushby et al, 2009: “Diagnosis and management of Duchenne muscular dystrophy, part 1: diagnosis, and pharmacological and psychosocial management” (The Lancet, Online publication, Nov. 30, 2009 DOI:10.1016/S1474−, 4422(09)70271-6) is a review that presents recommendations for DMD management based on analysis of independent expert ratings of assessments and interventions. Pages 10-11 of the review describe glucocorticoid regimens and dosing.

A typically prescribed dose range for a steroid as a monotherapy, in particular a glucocorticoid such as prednisone or prednisolone, is 0.3-1 mg/kg/day (suitably 0.7 or 0.75 mg/kg/day), or 0.3 mg/kg/day to 10 mg/kg/week, in humans.

A typically prescribed dose range for a VAP-1 inhibitor as a monotherapy in humans is 20-200 mg/day for carbidopa (suitably 30 mg/day or 75 mg/day), and 25-300 mg/day (suitably 25 mg/day or 50 mg/day) for benserazide.

In one embodiment, the VAP-1 inhibitor and the steroid are each prescribed at a dose that is within a typically prescribed dose range for each compound as a monotherapy. The compounds may be prescribed as separate dosages or as a combination dosage. Such combinations provide increased efficacy compared with the effect of either compound as a monotherapy.

In another embodiment, the VAP-1 inhibitor and the steroid are each prescribed at a dose that is below a typically prescribed dose for each component as a monotherapy, but at doses that have therapeutic efficacy in combination. The components may be prescribed as separate dosages or as a combination dosage. The dosages of the components in combination may be selected to provide a similar level of therapeutic efficacy as the VAP-1 inhibitor or the steroid as a monotherapy, but with the advantage that the lower doses of the VAP-1 inhibitor and/or the steroid reduce the risk of adverse side effects compared to the prescribed dosages of each compound as a monotherapy.

In another embodiment, the prescribed dosage of the VAP-1 inhibitor is within a typically prescribed dose range for monotherapy, and the steroid is prescribed at a dosage that is below a typically prescribed dose for monotherapy.

In a further embodiment, the prescribed dosage of the VAP-1 inhibitor is below a typically prescribed dose for monotherapy, and the steroid is prescribed at a dosage that is within a typically prescribed dose range for monotherapy.

Preferred dosages below the typically prescribed dose for monotherapy are doses that are up to 50%, or up to 25%, of the typically prescribed dose.

When administered in separate dosages, the VAP-1 inhibitor and the steroid may be administered substantially simultaneously (for example, within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, about 72 hours, or about 96 hours, or more.

The skilled person will be able to determine, and optimise, a suitable time course for sequential administration, depending on the particular combination of the VAP-1 inhibitor and the steroid. The time course is preferably selected such that there is at least one beneficial effect, for example an enhancing of the effect of the VAP-1 inhibitor or the steroid, or a mutual enhancing of the effect of the combination components, for example a more than additive effect, additional advantageous effects, fewer side effects, less toxicity, or a combined therapeutic effect compared with a non-effective dosage of one or both of the combination components, and very preferably a synergism of the combination components.

It will be appreciated that the optimum time course will depend on factors such as the time taken for the peak plasma concentration of the compound to be reached after administration, and the elimination half-life of each compound. Preferably the time difference is less than the half-life of the first component to be administered.

The skilled person will also be able to determine appropriate timing for administration. In certain embodiments, the VAP-1 inhibitor may be administered in the morning, and the steroid administered at least once later in the day. In other embodiments, the VAP-1 inhibitor and the steroid may be administered at substantially the same time.

The subject may receive doses of the VAP-1 inhibitor and the steroid over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more.

In general, the components of a combination of the invention may be administered by known means, in any suitable formulation, by any suitable route. Suitable routes of administration may include by oral, rectal, nasal, topical (including buccal and sublingual), sublingual, transdermal, intrathecal, transmucosal or parenteral (including subcutaneous, intramuscular, intravenous and intradermal) administration. In some embodiments, the VAP-1 inhibitor and the steroid are administered orally.

Suitable pharmaceutical compositions and dosage forms may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate combined preparations of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients:It should be noted that, in some cases, two or more individual dosage units in combination provide a therapeutically effective amount of the active agent, for example, two tablets or capsules taken together may provide a therapeutically effective dosage, such that the unit dosage in each tablet or capsule is approximately 50% of the therapeutically effective amount.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

In addition to the formulations described previously, the active agent may be formulated as a depot preparation for controlled release of the active agent, preferably sustained release over an extended time period. These sustained release dosage forms are generally administered by implantation (for example, subcutaneously or intramuscularly or by intramuscular injection).

Combined preparations of the invention may be packaged with instructions for administration of the components on the combination. The instructions may be recorded on a suitable recording medium or substrate. For example, the instructions may be printed on a substrate, such as paper or plastic. The instructions may be present as a package insert, in the labeling of the container or components thereof (i.e., associated with the packaging or sub-packaging). In other embodiments, the instructions are present as an electronic storage data file present on a suitable computer readable storage medium, for example, CD-ROM, diskette. Some or all components of the combined preparation may be packaged in suitable packaging to maintain sterility.

VAP-1 Inhibition Assay

This assay is performed at room temperature with purified recombinantly expressed human VAP-1 (SSAO). Enzyme was prepared essentially as described in Ohman et al. (Protein Expression and Purification 46 (2006) 321-331). The enzyme activity is assayed with benzylamine as substrate by measuring either benzaldehyde production, using 14C-labeled substrate, or by utilizing the production of hydrogen peroxide in a horseradish peroxidise (HRP) coupled reaction. Briefly, test compounds are dissolved in dimethyl sulfoxide (DMSO) to a concentration of 10 mM. Dose-response measurements are assayed by either creating 1:10 serial dilutions in DMSO to produce a 7 point curve or by making 1:3 serial dilutions in DMSO to produce 11 point curves. The top concentrations are adjusted depending on the potency of the compounds and subsequent dilution in reaction buffer yielded a final DMSO concentration≦2%.

Hydrogen peroxide detection: In a horseradish peroxidise (HRP) coupled reaction, hydrogen peroxide oxidation of 10- acetyl-3,7-dihydroxyphenoxazine produces resorufin, which is a highly fluorescent compound (Zhout and Panchuk-Voloshina. Analytical Biochemistry 253 (1997) 169-174; AmplexR Red Hydrogen Peroxide/peroxidise Assay kit, Invitrogen A22188). Enzyme and compounds in 50 mM sodium phosphate, pH 7.4 are set to pre-incubate in flat-bottomed microtiter plates for approximately 15 minutes before initiating the reaction by addition of a mixture of HRP, benzylamine and Amplex reagent. Benzylamine concentration is fixed at a concentration corresponding to the Michaelis constant, determined using standard procedures. Fluorescence intensity is then measured at several time points during 1-2 hours, exciting at 544 nm and reading the emission at 590 nm. For the human SSAO assay final concentrations of the reagents in the assay wells are: SSAO enzyme 1 mg/ml, benzylamine 100 μM, Amplex reagent 20 μM, HRP 0.1 U/mL and varying concentrations of test compound. The inhibition is measured as % decrease of the signal compared to a control without inhibitor (only diluted DMSO). The background signal from a sample containing no SSAO enzyme is subtracted from all data points. Data is fitted to a four parameter logistic model and IC50 values are calculated, for example by using the GraphPad Prism 4 or XLfit 4 programs.

Aldehyde Detection:

SSAO activity is assayed using 14C-labeled benzylamine and analysed by measuring radioactive benzaldehyde. In a white 96-well optiplate (Packard), 20 μL of diluted test compound is pre-incubated at it with 20 μL SSAO enzyme for approximately 15 minutes with continuous agitation. All dilutions are made with PBS. The reaction is initiated by adding 20 μL of the benzylamine substrate solution containing [7-14C] Benzylamine hydrochloride (CFA589, GE Healthcare). The plate is incubated for 1 hour as above after which the reaction is stopped by acidification (10 μL 1 M HCl). Then 90 μL Micro Scint-E solution (Perkin-Elmer) is added to each well and the plate is continuously mixed for 15 minutes. Phase separation occurs and activity is read in a scintillation counter (eg Topcount, Perkin-Elmer). In the final reaction well, human recombinant SSAO concentration is 10 μg/ml. In order to optimize sensitivity, the substrate concentration is decreased as compared to the HRP coupled assay in order to get a higher fraction of radioactive product. In the human SSAO assay, benzylamine concentration is 40 μM (0.2 Ci/mL). Data is analysed as above.

Embodiments of the invention are described below, with reference to the accompanying drawings.

### EXAMPLE 1

Studies in to the overexpression of VAP-1 in dystrophic muscle tissue are on-going in tissue sections derived from patients with muscular dystrophy.

In these on-going studies, the increased expression of VAP-1 in the tissue section (detected with a goat anti-human VAP-1 antibody (Everest) followed by Cy3 labelled anti-goat IgG and imaged using a confocal microscope) and a monoclonal rat anti mouse antibody followed by a Cy3 labelled anti-rat antibody is revealed when compared to non-dystrophic control tissue.

In further on-going experiments the effect of VAP-1/SSAO inhibitors including carbidopa is being examined in the mdx and dy/dy mouse models of muscular dystrophy. In these models groups of mice were dosed once per day with carbidopa (25 mg/kg p.o.) for up to 12 weeks. The degree of inflammation and fibrosis in the muscle was then examined.

### EXAMPLE 2

### VAP-1 Expression is Increased in the Muscle of a Patient with Duchenne Muscular Dystrophy (DMD)

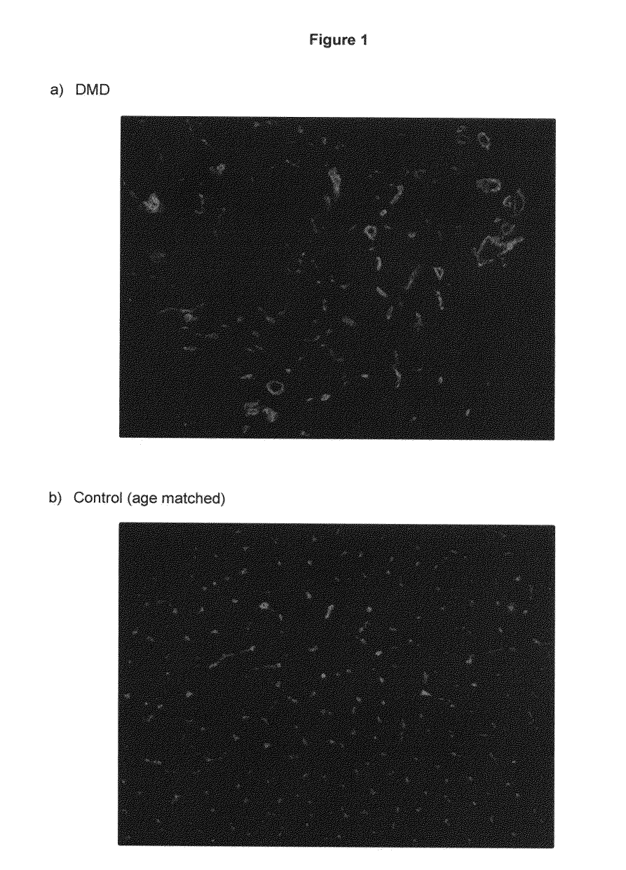

The expression of VAP-1 in a muscle tissue section of a boy with Duchenne Muscular Dystrophy (DMD) was compared with VAP-1 expression in a muscle tissue section of an age-matched boy with normal muscles as a control. VAP-1 expression was detected with a monoclonal rat anti-mouse VAP-1 antibody, followed by a Cy3-labelled anti-rat IgG antibody, and imaged using a confocal microscope. The results are shown in FIG. 1.

FIG. 1(a) shows VAP-1 expression in the DMD tissue section, and FIG. 1(b) shows VAP-1 expression in the age-matched control. VAP-1 expression is greatly increased in the DMD tissue section.

### EXAMPLE 3

### Effect of the VAP-1 Inhibitor Benserazide on Diaphragm Muscle in a Mouse Model of Muscular Dystrophy

Duchenne muscular dystrophy (DMD) is an X-linked muscle disease. Patients develop progressive weakness of skeletal and respiratory muscles and dilated cardiomyopathy. Clinical onset is usually between 2 and 5 years of age. Most patients loose independent ambulation in their teens, after which scoliosis develops. Death usually occurs before forty years of age and is most often the result of respiratory or cardiac failure. The biochemical cause of DMD is a severe deficiency of dystrophin, an essential component of the sarcolemmal dystrophin-associated glycoprotein complex. When complex assembly is disturbed, the linkage between the muscle cell's cytoskeleton and the extracellular matrix is compromised, leading to sarcolemmal instability and increased vulnerability to mechanical stress. Fibres undergo necrosis by excessive Ca²⁺ influx and are progressively replaced by connective and adipose tissue.

The immune system plays a pivotal role in the pathogenesis of DMD. Contraction of dystrophin deficient myofibres produces severe damage and generates cycles of muscle fibre necrosis and regeneration. Necrotizing myofibres are attacked by macrophages; inflammatory cells are present throughout the endomysial, perimysial, and perivascular areas. Macrophages are the most abundant immune cells observed in DMD muscle and both proinflammatory M1 phenotype macrophages and regeneration-focussed M2 phenotype macrophages are present. Within the inflammatory areas, few T cells, B cells, and dendritic cells are also present. Infiltrating T cells are predominantly CD4+, and smaller numbers of CD8+Tcells can be found. The T cell receptor repertoire of CD4+ and CD8+ T cells does not display dominant Va or Vβ rearrangements, which points toward a nonspecific cell recruitment to sites of muscle fibre destruction. In addition to their involvement in muscle damage, T cells also play an important role in the fibrotic processes present in dystrophic muscle. T cell deficiency significantly reduces collagen matrix accumulation in the murine model. The build up of the inflammatory response is regulated through interactions between adhesion molecules, receptors, and soluble factors, recruiting immune cells from the blood stream to the muscle tissue.

The most studied animal model for DMD is the mdx mouse. This was first described by Bulfield et al (Proc. Natl. Acad. Sci. USA, 1984, 81:1189-1192). It has a point mutation within its dystrophin gene, and as a result the mouse has no functional dystrophin in its muscles. Early in life, the mdx mouse exhibits phases of marked skeletal muscle degeneration and subsequent regeneration. As it ages, certain muscle types (including the diaphragm) show weakness and increased fibrosis. The mdx mouse diaphragm reproduces the degenerative changes of DMD, exhibiting a pattern of degeneration, fibrosis and severe functional deficit comparable to that of DMD limb muscle. This provides a quantitative framework for studying the pathogenesis of dystrophy (Stedman et al, Nature, 1991, 352, 536-539).

12 week old mdx mice were treated with benserazide (20 mg/kg, po, once per day) or vehicle (water, once per day), in groups of 8 mice. After 6 weeks of treatment, diaphragms of the mice were collected and flash frozen in liquid nitrogen-cooled isopentane. The sections were stored on slides at −20° C. until required.

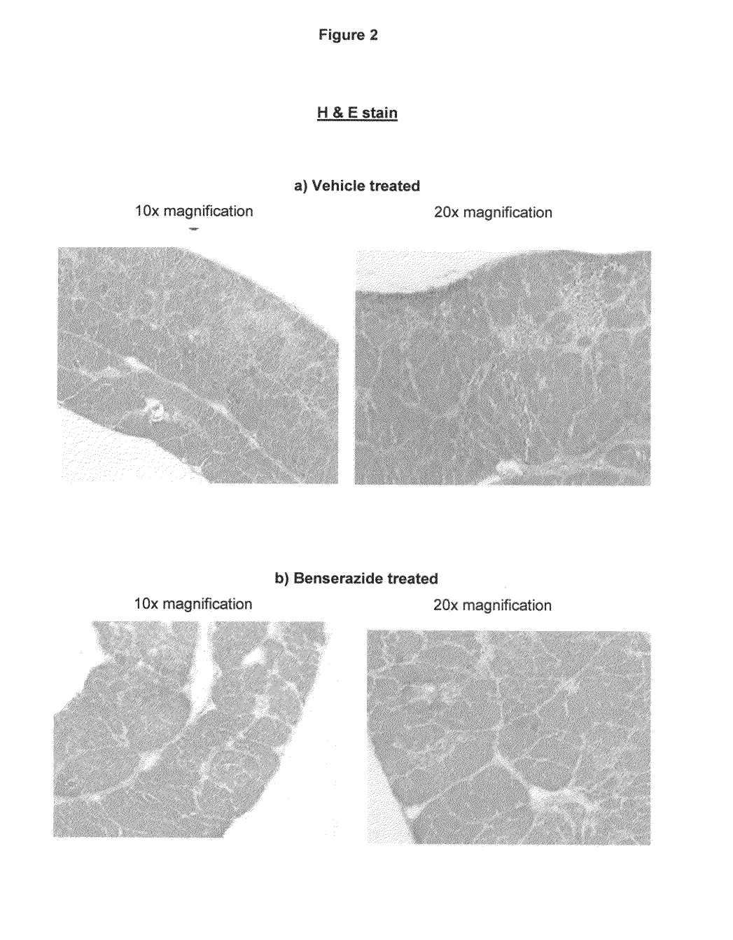

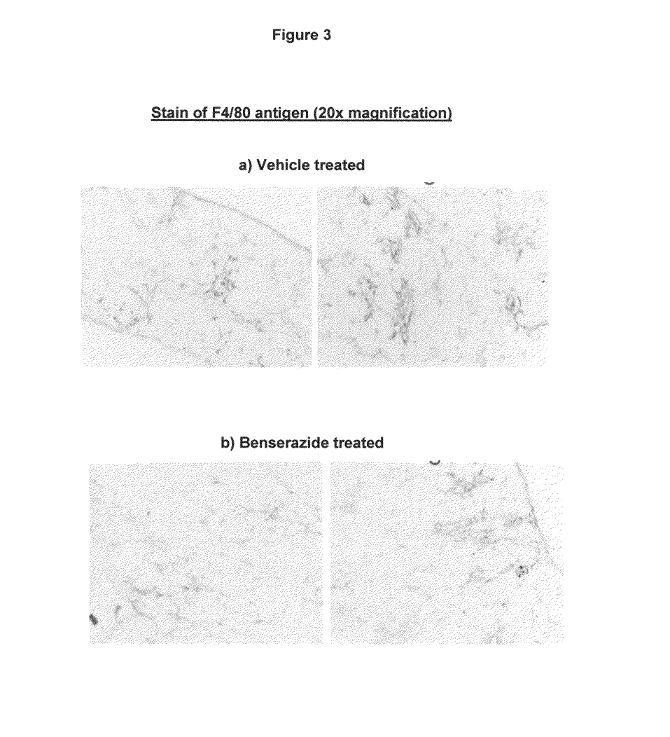

Hematoxylin and eosin (H & E) staining was used to show cytoplasmic, nuclear, and extracellular matrix features. Hematoxylin stains nucleic acids, and eosin stains proteins nonspecifically. Staining of F4/80 antigen (a glycoprotein expressed by murine macrophages) was used to show macrophages. The results of H & E staining are shown in FIG. 2, and the results of staining of murine F4/80 antigen are shown in FIG. 3.

The H & E staining in FIG. 2 shows an approximate 50% reduction in inflammatory infiltrates in mice treated with benserazide compared to vehicle. The F4/80 staining in FIG. 3 also shows an approximate 50% reduction in macrophage infiltration in mice treated with benserazide compared to vehicle.

These results show that the VAP-1 inhibitor benserazide reduces the inflammatory response to muscle damage in dystrophic mice. It is known from the mdx mouse model that partial inhibition of macrophage incursion into the muscle tissue has a beneficial effect on muscle tissue maintenance. Thus, this example shows that the VAP-1 inhibitor benserazide can be used for the treatment of dystrophic muscle, and muscular dystrophy.

### EXAMPLE 4

### Effect of Prednisolone, Carbidopa, and Benserazide on Myeloid and Lymphoid Cell Recruitment in Muscles of mdx Mice

Glucocorticoids are the only medication currently clinically available that slows the decline in muscle strength and function in DMD, which in turn reduces the risk of scoliosis and stabilises pulmonary function. The glucocorticoid prednisolone is often used in Europe to treat DMD. However, there are serious side effects associated with chronic administration of glucocorticoids.

This example describes the results of a comparison of the effects of prednisolone, and the VAP-1 inhibitors carbidopa and benserazide, on infiltration of myeloid and lymphoid cells into muscles of mdx mice.

Introduction

Madaro & Bouché (BioMed Research International, 2014, Article ID 438675) describe the contribution of immune cells in the onset and progression of muscle dystrophies, particularly DMD. Only a small number of immune cells reside within intact skeletal muscle, but they are recruited during injury and play important roles in the regeneration process. Upon injury, immune cells rapidly infiltrate the muscle to remove necrotic tissue and secrete soluble factors that activate muscle satellite cells (MuSCs). Satellite cells and immune cells attract one another through chemokines. Satellite cells secrete proinflammatory cytokines, such as IL-1, IL-6, and TNF-α to facilitate immune cell infiltration and function. Immune cells secrete diffusible factors, such as growth factors, IL-6, globular adiponectin, extracellular matrix (ECM) components, and ECM remodeling matrix metalloproteinases (MMPs). These diffusible factors generate ECM chemoattractive fragments, which help satellite cells escape from the basal lamina of myofibres, and promote satellite cell proliferation. In addition, cell-to-cell contact between immune and satellite cells protects satellite cells from apoptosis. Disruption of these events lead to impaired regeneration, increased muscle wasting, and deposition of fibrotic tissue, as occurs in muscular dystrophies, such as Duchenne muscular dystrophy (DMD).

Dystrophin is a critical component of the dystrophin glycoprotein complex (DGC), acting as a link between the cytoskeleton and extracellular matrix in skeletal and cardiac muscles. Inefficiency of the DGC in DMD causes muscle fragility, contraction-induced damage, necrosis, and inflammation. Although satellite cells compensate for muscle fibre loss in the early stages of disease, eventually these progenitors become exhausted. Moreover, aberrant intracellular signalling cascades that regulate both inflammatory and immune processes contribute substantially to the degenerative process. As a result, fibrous and fatty connective tissue overtakes the functional myofibres. These changes culminate in progressive muscle wasting, with the majority of patients being wheelchair-bound in their early teens, succumbing to cardiac/respiratory failure in their twenties.

The mdx mouse model of DMD exhibits extensive limb muscle degeneration and inflammation, as well as myocardial lesions. Although lack of dystrophin makes myofibres susceptible to fragility and degeneration when contracting, this mechanical defect hypothesis for dystrophic muscle death has been unable to explain many aspects of the pathophysiology of DMD. Early immune cell infiltration in DMD patients and mdx mice is believed to represent an important aspect of dystrophic muscle pathology.

DMD muscle is characterized by continuous cycles of necrosis and repair of myofibers. Myofibers undergoing degeneration/necrosis, independently of the injury insult, release Th1 inflammatory stimuli, which recruit neutrophils and monocytes/macrophages required to clear cell debris, followed by a Th2 immune response which promotes muscle healing.

Neutrophils

In acute injury, neutrophils are the first cells to invade injured muscle, followed by macrophages. In acutely injured muscle in mice, they begin to appear at elevated numbers within 2 hours of muscle damage, typically peaking in concentration between 6 and 24 hours after injury, and then rapidly decline in numbers. Their function mostly involves phagocytic activity to remove debris but also release of TNFα, as a Th1 stimuli, and production of myeloperoxidase (MPO), inducing muscle membrane damage and increasing macrophage proinflammatory activity.

As in acute injury, neutrophils, together with macrophages, invade mdx dystrophic muscle as early as 2 weeks of age. Initial muscle injury and membrane lysis are caused by superoxide production mediated by these early infiltrating neutrophils.

Previous studies have shown that anti-GR1 antibody mediated depletion of neutrophils, starting at age 19 days, significantly reduces muscle necrosis at age ˜21 days and subsequent regeneration at age ˜28 days.

Monocytes

Monocytes can differentiate into inflammatory or anti-inflammatory subsets. Inflammatory monocytes selectively traffic to the sites of inflammation, produce inflammatory cytokines and contribute to local and systemic inflammation. They are highly infiltrative and can be differentiated into inflammatory macrophages, which remove pathogen-associated molecular patterns (PAMPs) and cell debris. Anti-inflammatory monocytes patrol the vasculature to monitor PAMPs and become tissue resident macrophages. During inflammation, they differentiate into anti-inflammatory macrophages, which repair damaged tissues.

Mouse monocyte subsets are characterized by differential expression of an inflammatory monocyte marker Ly6C (Gr1) (Yang et al, Biomarker Research 2014, 2:1-9). Mouse monocyte subsets are grouped as pro-inflammatory Ly6C⁺ (further divided as Ly6C^{high} and Ly6C^{middle}) and anti-inflammatory Ly6C⁻ (also called Ly6C^{low}) monocyte subsets based on expression levels of Ly6C on the cell surface. Mouse Ly6C⁺ monocytes have a high antimicrobial capability due to their potent capacity for phagocytosis, secrete ROS, TNFα, nitric oxide, IL-1β, a little amount of IL-10 upon bacterial infection, and a large amount of type 1 interferon (IFN) in response to viral ligands. CCR2-CCL2 signaling in Ly6C⁺ monocytes alters the conformational change of VLA-4 (α4β1 integrin), the ligand for VCAM-1, leading to high affinity interaction and monocyte transmigration. In vascular inflammation, Ly6C⁺ monocytes are preferentially recruited into inflamed tissue via interaction of chemokine receptor CCR2 and are more likely to mature to inflammatory M1 macrophages, which are distinguished by secretion of pro-inflammatory cytokine, TNFα, and IL-6 and contribute to tissue degradation and T cell activation.

In steady state, Ly6C⁺ monocytes differentiate into Ly6C⁻ monocytes in the circulation. This subset patrols the luminal side of endothelium of small blood vessels and bind to endothelium by chemokine receptor CX3CR1 via LAF-1/ICAM1-dependent manner. The patrolling behavior of monocytes may be due to low-level expression of adhesion molecules. Ly6C⁻ monocytes secrete anti-inflammatory cytokine, IL-10 upon in vivo bacterial infection. In vascular inflammation, Ly6C⁻ monocytes are recruited to tissue and are more likely to differentiate into M2 macrophages, which secrete anti- inflammatory cytokine and contribute to tissue repair.

Macrophages

Two subpopulations of macrophages have been identified in regenerating muscle tissue that may influence muscle degeneration and regeneration depending on the proportion of these cells present. The M1 population are proinflammatory, characterized by the expression of iNOS and secretion of proinflammatory cytokines (e.g., TNFα, IL-1β, and IL-6), and promote muscle cell lysis. By contrast, the M2 population is characterized by the expression of arginase-1, CD163, and CD206 mannose receptor (usually in noninflammatory, repair conditions) and/or anti-inflammatory cytokines (e.g., IL-10). They are believed to enhance muscle regeneration, by inducing satellite cell proliferation.

However, macrophages exhibit a wide variety of intermediate phenotypes, including M2b and M2c, M1 and M2 being the extremes of a continuum in activation states. M2b macrophages are known to release large amounts of IL-10, which promotes the proliferation of nonmyeloid cells, although, like M1 macrophages, they can also release proinflammatory cytokines, such as IL-1β and TNFα. IL-10 can also induce M2c macrophages, which have anti-inflammatory functions.

In acute muscle injury the sequential waves of M1, followed by M2 macrophage invasion, leads to resolution of inflammation and efficient muscle repair. However, in muscular dystrophies, repetitive cycles of myofiber degeneration lead to muscle invasion by M1 macrophages together with M2a macrophages, which may reduce the cytotoxic activity of M1 macrophages. Subsequent invasion of dystrophic muscle by M2c macrophages is associated with progression to the regenerative phase in pathophysiology. The number of M2 macrophages declines upon resolution of the damage in acute-injured muscle, but in mdx muscle their number increases with age, and promotes fibrosis. Thus, increased and persistent presence of macrophages modifies the intensity, duration, and interactions of the different released factors, leading to increased necrosis of myofibers, ECM accumulation, and replacement of muscle with fibrotic and fat tissue.

Previous studies have shown that depletion of macrophages from mdx mice before the onset of histopathology caused great reduction in muscle pathology in 4-week-old mice.

Eosinophils

Eosinophil invasion has been found in both DMD and mdx dystrophies. Previous studies observed that eosinophils increased within mdx dystrophic muscle at about 4 weeks of age, together with cytotoxic T cells invasion, and, although their number decreases during the regenerative phase, their concentration remains higher at 30-32 weeks of age, as compared to healthy muscle of age-matched wild type mice, depending on the muscle examined. Prednisone treatment has been shown to reduce eosinophil infiltration.

T-Cell Response in DMD.

It is widely believed that lymphocytes do not play a relevant role in healthy regenerating muscle, due to the inability of skeletal muscle to activate a T cell response. Neither MHC class I nor class II molecules has been detected on muscle fibers from healthy muscle tissues. By contrast, appearance of MHC class I and/or II was observed in muscle tissue of patients with idiopathic inflammatory myopathies (IIM), where an autoimmune pathogenesis is now recognized, but also in regenerating fibers of patients with DMD. T cells are found in degenerating muscle after acute injury, but their recruitment is more robust and persistent in chronic diseases, such as muscular dystrophies. Persistence of T cells in dystrophic muscle may actually modulate inflammatory milieu and immune cell activity, but may also directly interfere with muscle cell function through lymphocyte-released cytokines and chemokines.

Very early studies correlated the reduction in T cells observed in prednisone-treated DMD patients, with reduction in muscle necrosis and fibrosis. Further studies identified T cells in muscles of several DMD patients, characterized by a specific T-cell receptor (TCR) rearrangement. The over-representation of a T-cell population expressing a restricted set of TCR variable genes might indicate a selective T-cell response directed to a muscle-specific antigen. Their persistence in DMD muscle could derive from either clonal expansion or conserved antigen recognition, or from the emergence of a regulatory population.

T-Cell Response in mdx Muscle.

Elevated concentrations of activated cytotoxic CD8⁺ and helper CD4⁺ T cells (Th) are present in affected muscles of mdx mice aged 4-8 weeks but rapidly decrease in concentration by 14 weeks of age. CD8⁺ T cells are the first to invade dystrophic muscle, peaking at 4 weeks of age; their activation is generally driven by a Th1 cellular immune response to kill their target cells through perforin-mediated processes. Around 2 weeks later, CD4⁺ T cells also invade dystrophic muscle; T helper CD4⁺ T cells can generally differentiate into Th effector inflammatory cells, mainly Thb1 and Th2, or into regulatory T cells (Treg), both of which participate in immune responses. Th1 cells are known to support macrophage M1 polarization by producing IL-1, IL-2, TNF-α, and INF-γ, while Th2 produce IL-4, IL-13, and IL-6 sustaining the M2 macrophage polarization. Treg cells are required for the resolution of the immune response. These cells produce anti-inflammatory cytokines such as IL-10.

One of the first studies addressing the possible role of lymphocytes in mdx showed that antibody-mediated depletion of CD8⁺ or CD4⁺ cells in mdx mice, beginning at 6 days of age and continuing until the age of 4 weeks, resulted in a 75% and 61% reduction in muscle histopathology, respectively. This positive outcome suggested an important role for these cells in the development of muscle lesions. In another study, scid/mdx mice, which are deficient in functional T and B lymphocytes, were shown to develop much less diaphragm fibrosis at 1 year of age and a decrease in activated TGFβ in skeletal muscle, compared with mdx mice. Improvement in muscle regeneration was also observed in these mice, but not in muscle functionality. Accordingly, in nu/nu/mdx mice, the lack of functional T cells alone was associated with less diaphragm fibrosis at 3 months of age. Altogether, these results support the pathogenic role of T cells in mdx muscle and reveal this lymphocyte subclass as an important source of TGFβ1.

T cells represent approximately 3% of all infiltrating cells in mdx muscle, with over half present as double-negative T cells (lacking both CD4 and CD8 expression), 8%-10% of which were recently identified as being NKT-like cells, which express both T and NK markers.

Treatment with the immunosuppressant drug Rapamycin ameliorates the mdx phenotype with a reduction of both CD4⁺ and CD8⁺ cells.

In this example, labelled antibodies were used to assess infiltration of inflammatory myeloid and lymphoid cells into muscles of mdx mice following treatment of the mice with different doses of the glucocorticoid prednisolone, and the VAP-1 inhibitors carbidopa and benserazide. CD11b is a transmembrane glycoprotein which is expressed on the surface of granulocytes (including neutrophils and eosinophils), monocytes, NK cells, dendritic cells, tissue macrophages and subsets of T and B cells. Labelled antibody to CD11b was used to assess infiltration of inflammatory myeloid cells into muscles of mdx mice. Labelled antibody to Ly6C was used to assess infiltration of pro- and anti-inflammatory monocytes into muscles of mdx mice. Labelled antibody to CD4 and CD8a antigen was used to assess infiltration of Th cells (CD4⁺CD8⁻) and double negative (CD4⁻CD8⁻) T cells into muscles of mdx mice.

Mice and Housing

A total of a ninety C57BU10ScSn-Dmd_{mdx}/J male mice, 3 weeks of age (+/−3days), were transferred to the in vivo research laboratory in Bar Harbor, Me. Additional ten C57BU10ScSnJ male mice 3 weeks of age (+/−3 days) were also transferred and served as a wild-type control group. The mice were ear notched for identification and housed in ventilated polysulfone cages with HEPA filtered air at a density of 3-4 mice per cage. The animal room was lit entirely with artificial fluorescent lighting on controlled 12 hour light/dark cycle (6 a.m. to 6 p.m. light). The normal temperature and relative humidity ranges in the animal rooms were maintained at 22±4° C. and 50±15%, respectively. The animal room was set for 15 air exchanges per hour. Filtered tap water acidified to a pH of 2.8 to 3.2 was provided ad libitum. LabDiet 5K52 was provided ad libitum.

Methods

Prior to study initiation, mice were acclimated for 3 days. Mice were tested for grip strength and in the open field at ages 25 and 26 days, and drug regimen was initiated at age 27 days.

Treatment Protocols

Mice were monitored twice weekly for clinical observations and body weights. Mice in each group were treated as follows, for 4 weeks:
Prednisolone: 0.2 and 1 mg/kg ip once per day;
Benserazide: 7 and 20 mg/kg po twice per day;
Carbidopa 50 and 100 mg/kg po twice per day;
Carbidopa plus prednisolone: 100 mg/kg carbidopa po twice per day, and 1 mg/kg prednisolone ip
Vehicle: ip and po

At 8 weeks of age, mice were tested again for grip test and in the open field.

Sample Collection

24 hours after the second open field tests, blood was collected by retro-orbital bleeding, and mice were humanely euthanized by CO₂ asphyxiation. One triceps surae was collected fresh and processed for immune cell sorting, one tibialis anterior was preserved in RNALater for RNA extraction, one whole hind limb and the diaphragm were fixed in 2% paraformaldehyde overnight at 4° C. for paraffin embedding and histology stains.

Serum was prepared immediately after collection and frozen at −20° C. At the end of the study, all serum samples were dosed for Creatine Kinase on a Beckman Coulter AU Clinical Chemistry analyzer following manufacturer instructions (a modification of the International Federation of Clinical Chemistry method).

Cell Sorting

For immune cell sorting, muscle samples were enzymatically and mechanically dissociated following manufacturer instructions (Skeletal Muscle Dissociation KitCatalog no. 130-098-305, Miltenyi Biotec). Cells were stained with the following antibodies:

| | | Cell type |
|---|---|---|
| Antigen | Clone | (for illustration purposes) |
| CD4 (L3/T4) | GKL5 | T helper |
| CD8a | 53-6.72 | T Cytotoxic |
| CD11b/MAC-1 | M1/70 | Monocytes |
| GR-1/Ly6G | RB6-8C | Neutrophils |
| CD45R (B220) | RA3-6B2 | Leukocytes |
| Ly6C | AL-21 | Pro-/anti-inflammatory |
| | | monocytes |

Stained cells were analyzed on a BD LSR II cytometer and counts of different populations were expressed in percentage of the number of live cells counted.

mRNA Quantification (RT PCR)

Total RNAs were extracted with a modification of the Trizol method, reverse transcribed, and mRNAs of TGFβ, and the following inflammation markers were quantified by the SYBR green method with GAPDH as a normalizer:

| mRNA | Cell type/event |
|---|---|
| TNF-α | Pro-inflammatory cytokine |
| Interleukin 6 | Pro-inflammatory cytokine |
| Mpeg1 | Macrophage |
| Lgals3 | Macrophage |
| CD53 | B/T cells |
| CD48 | B/T cells activation |
| Ly6c | Pro-/anti-inflammatory monocytes |
| CD11b/MAC-1 | Monocytes |

Results

### Effect of Prednisolone, Benserazide, and Carbidopa on Recruitment of CD11b⁺ Myeloid Cells into Muscles of mdx Mice

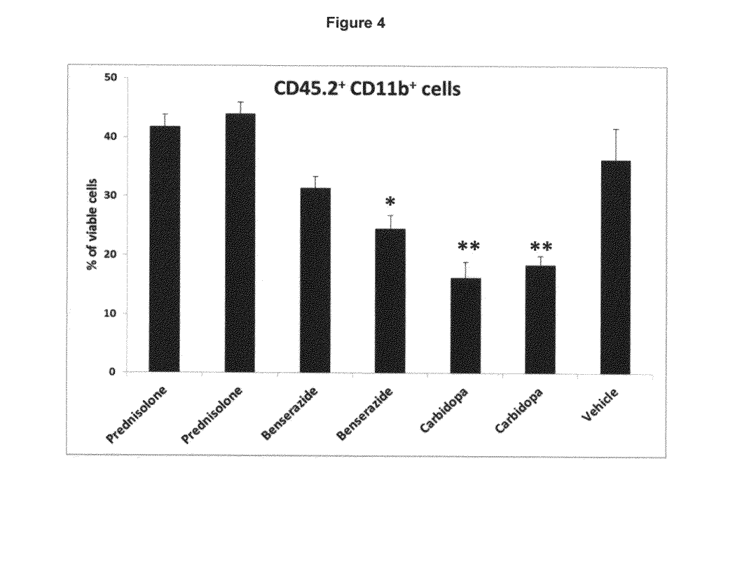

The effects of prednisolone, benserazide, and carbidopa, on recruitment of CD11b⁺ myeloid cells into muscles of mdx mice are shown in the Table below, and in FIG. 4.

| | | | % change |
|---|---|---|---|
| | | | compared to |
| Drug | Dose | % of viable cells | vehicle |
| Prednisolone | 0.2 mg/kg ip once/day | 41.8 | +14.8 |
| | 1 mg/kg ip once/day | 44 | +20.9 |
| Benserazide | 7 mg/kg po twice/day | 31.4 | −13.7 |
| | 20 mg/kg po twice/day | 24.6* | −32.4 |
| Carbidopa | 50 mg/kg po twice/day | 16.3** | −55.2 |
| | 100 mg/kg po twice/day | 18.5** | −49.2 |
| Vehicle | — | 36.4 | 0 |

*P < 0.05, **P < 0.005, compared to vehicle

The results show that the VAP-1 inhibitors benserazide, and carbidopa in particular, dramatically reduced CD11b⁺ myeloid cell recruitment. Benserazide reduced CD11b⁺ myeloid cell recruitment compared with vehicle (compare columns 3 and 4 with column 7 of FIG. 4). This effect was dose-dependent. At the higher dose of benserazide, CD11b⁺ myeloid cell recruitment was reduced by up to approximately one-third. Carbidopa reduced myeloid cell recruitment by approximately 50% compared with vehicle (compare columns 5 and 6 with column 7 of FIG. 4).

It was concluded from these results that the VAP-1 inhibitors benserazide and carbidopa reduced recruitment of inflammatory myeloid cells into the muscle of mdx mice.

### Effect of Prednisolone, Benserazide, and Carbidopa on Recruitment of Profibrotic and Anti-Fibrotic Myeloid Cells into Muscles of mdx Mice

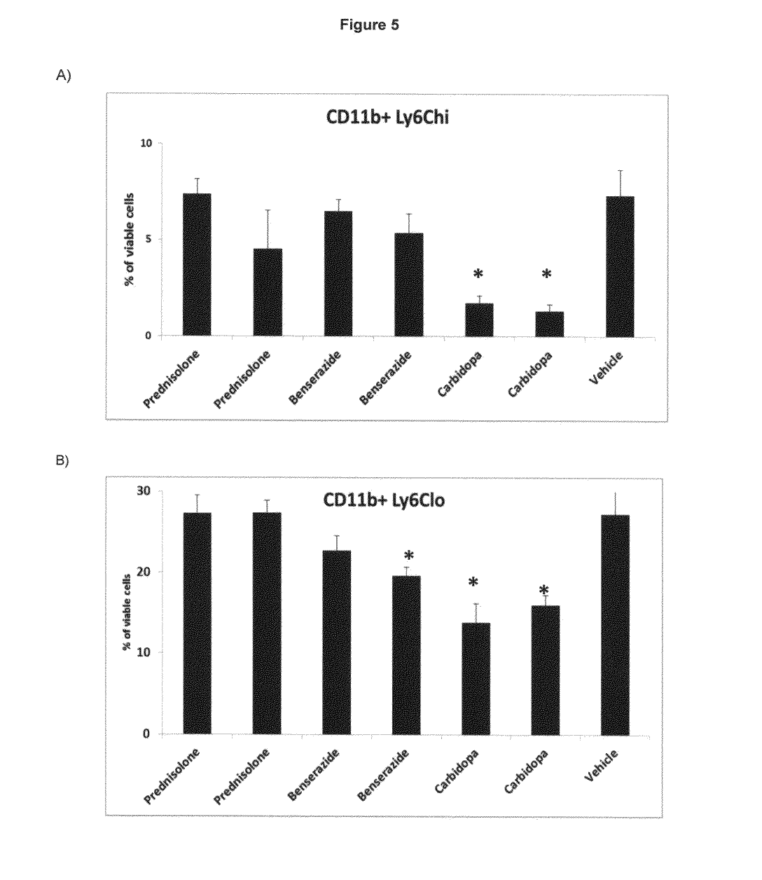

The effects of prednisolone, benserazide, and carbidopa on recruitment of CD11b+Ly6C^{high} profibrotic myeloid cells (Ly6C⁺ monocytes) into muscles of mdx mice are shown in the Table below, and in FIG. 5A:

| | | | % change |
|---|---|---|---|
| | | | compared to |
| Drug | Dose | % of viable cells | vehicle |
| Prednisolone | 0.2 mg/kg ip once/day | 7.37 | +0.6 |
| | 1 mg/kg ip once/day | 4.52 | −38.3 |
| Benserazide | 7 mg/kg po twice/day | 6.49 | −11.5 |
| | 20 mg/kg po twice/day | 5.36 | −26.9 |
| Carbidopa | 50 mg/kg po twice/day | 1.75** | −86.4 |
| | 100 mg/kg po twice/day | 1.31** | −82.1 |
| Vehicle | — | 7.33 | 0 |

**P < 0.005, compared to vehicle

The effects of prednisolone, benserazide, and carbidopa on recruitment of CD11b+Ly6C^{low} anti-fibrotic myeloid cells (Ly6C⁻ monocytes) into muscles of mdx mice is shown in the Table below, and in FIG. 5B:

| | | | % change |
|---|---|---|---|
| | | | compared to |
| Drug | Dose | % of viable cells | vehicle |
| Prednisolone | 0.2 mg/kg ip once/day | 27.3 | +0.4 |
| | 1 mg/kg ip once/day | 27.4 | +0.7 |
| Benserazide | 7 mg/kg po twice/day | 22.7 | −16.5 |
| | 20 mg/kg po twice/day | 19.6* | −27.9 |
| Carbidopa | 50 mg/kg po twice/day | 13.8** | −49.3 |
| | 100 mg/kg po twice/day | 16** | −41.2 |
| Vehicle | — | 27.2 | 0 |

*P < 0.05, **P < 0.005, compared to vehicle

The ratio of CD11b+Ly6C^{high} profibrotic myeloid cells to CD11b+Ly6C^{low} anti-fibrotic myeloid cells in mdx mouse muscle following treatment of mdx mice with prednisolone, benserazide, and carbidopa is shown below:

| | | Ratio of CD11b⁺Ly6C^{high} |
|---|---|---|
| | | profibrotic:CD11b^{+Ly6C}^{low} |
| Drug | Dose | anti-fibrotic myeloid cells |
| Prednisolone | 0.2 mg/kg ip once/day | 1:3.7 |
| | 1 mg/kg ip once/day | 1:6.1 |
| Benserazide | 7 mg/kg po twice/day | 1:3.5 |
| | 20 mg/kg po twice/day | 1:3.7 |
| Carbidopa | 50 mg/kg po twice/day | 1:7.9 |
| | 100 mg/kg po twice/day | 1:12.2 |
| Vehicle | — | 1:3.7 |

The results show that prednisolone appeared to have no statistically significant effect, compared with vehicle, on recruitment of profibrotic or anti-fibrotic myeloid cells (compare columns 1 and 2 with column 7 of FIG. 5A and FIG. 5B). Benserazide appeared to have little, if any, statistically significant effect, compared with vehicle, on recruitment of profibrotic myeloid cells (compare columns 3 and 4 with column 7 of FIG. 5A), but did show a statistically significant inhibition (27.9%) compared with vehicle on recruitment of anti-fibrotic myeloid cells at the higher dose (compare column 4 of FIG. 5B with column 7). However, the data shows that the relative levels of pro- and anti-fibrotic myeloid cells were not affected by either dose of benserazide.

Carbidopa dramatically inhibited recruitment of profibrotic myeloid cells into the muscles of mdx mice. Carbidopa reduced profibrotic myeloid cell recruitment by over 80% compared with vehicle at both doses tested (compare columns 5, 6 of FIG. 5A with column 7). Carbidopa also inhibited recruitment of anti-fibrotic myeloid cells into the muscles of mdx mice, but by less than 50% compared with vehicle (compare columns 5, 6 of FIG. 5B with column 7).

It was concluded from these results that carbidopa changed the relative levels of pro- and anti-fibrotic myeloid cells in mdx mouse muscle in favour of anti-fibrotic myeloid cells in a dose-dependent manner.

### Effect of Benserazide, Carbidopa, and a Combination of Carbidopa and Prednisolone, on Recruitment of Lymphoid CD4⁺ CD8⁻ and CD4⁻CD8⁻ T cells into muscles of mdx mice

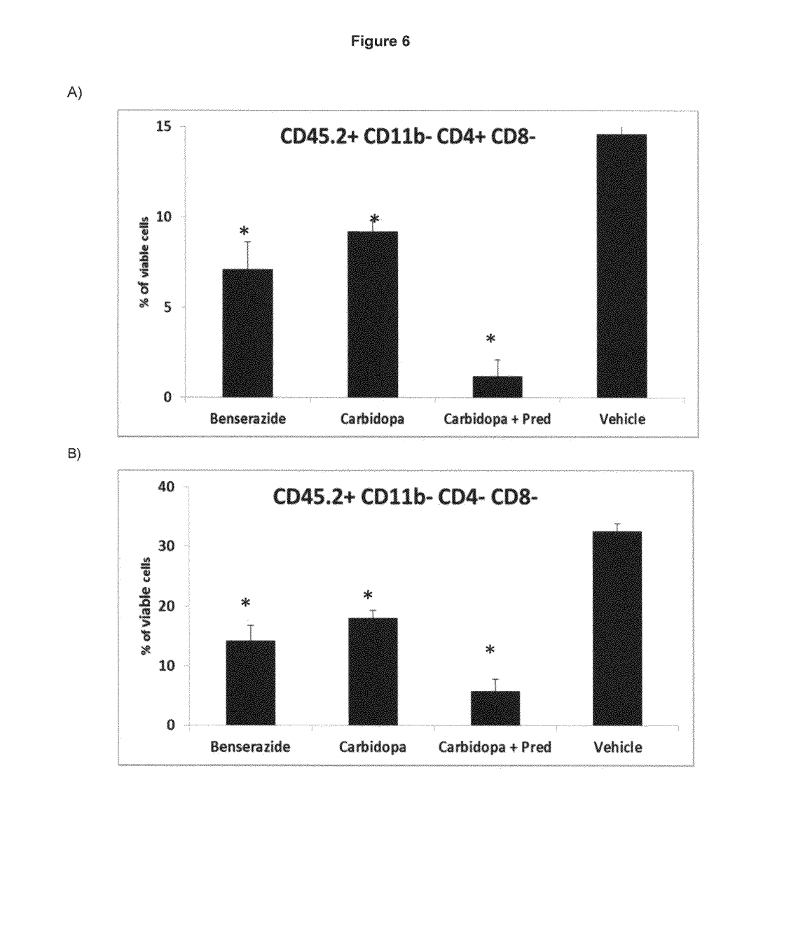

The effects of benserazide, carbidopa, and a combination of carbidopa and prednisolone, on recruitment of lymphoid CD4⁺CD8⁻ T cells into muscles of mdx mice are shown in the Table below, and in FIG. 6A:

| | | | % change |
|---|---|---|---|
| | | | compared to |
| Drug | Dose | % of viable cells | vehicle |
| Carbidopa | 100 mg/kg po twice/day | 7.1** | −51.4 |
| Benserazide | 20 mg/kg po twice/day | 9.2* | −37.0 |
| Carbidopa + | 100 mg/kg po twice/day | 1.2** | −91.8 |
| prednisolone | 1 mg/kg ip once/day | | |
| Vehicle | — | 14.6 | 0 |

*P < 0.05, **P < 0.005 compared to vehicle

The effects of benserazide, carbidopa, and a combination of carbidopa and prednisolone, on recruitment of lymphoid CD4⁻CD8⁻ (double negative, DN) T cells into muscles of mdx mice are shown in the Table below, and in FIG. 6B:

| | | | % change |
|---|---|---|---|
| | | | compared to |
| Drug | Dose | % of viable cells | vehicle |
| Carbidopa | 100 mg/kg po twice/day | 14.2** | −56.4 |
| Benserazide | 20 mg/kg po twice/day | 18** | −44.8 |
| Carbidopa + | 100 mg/kg po twice/day | 5.7** | −82.5 |
| prednisolone | 1 mg/kg ip once/day | | |
| Vehicle | — | 32.6 | 0 |

**P < 0.005 compared to vehicle

The results show that benserazide reduced recruitment of lymphoid CD4+CD8⁻ T cells, and lymphoid DN T cells, into muscles of mdx mice by over 50%, compared with vehicle. Carbidopa reduced recruitment of lymphoid CD4⁺CD8⁻ T cells by over 33.3%, and lymphoid DN T cells by nearly 50%, into muscles of mdx mice, compared with vehicle. The combined effects of carbidopa and a corticosteroid (prednisolone) were dramatic, reducing recruitment of lymphoid CD4⁺CD8⁻ T cells by over 90%, and lymphoid DN T cells by over 80%, compared with vehicle.

It was concluded from these results that treatment with carbidopa and benserazide reduced recruitment of T helper cells, and DN T cells, into the muscle of mdx mice. The effects of carbidopa were significantly enhanced by co-administration of carbidopa with a glucocorticoid.

### Effect of Prednisolone, Benserazide, Carbidopa, and a Combination of Carbidopa and Prednisolone, on the mRNA Level of Inflammatory Markers in Muscles of mdx Mice

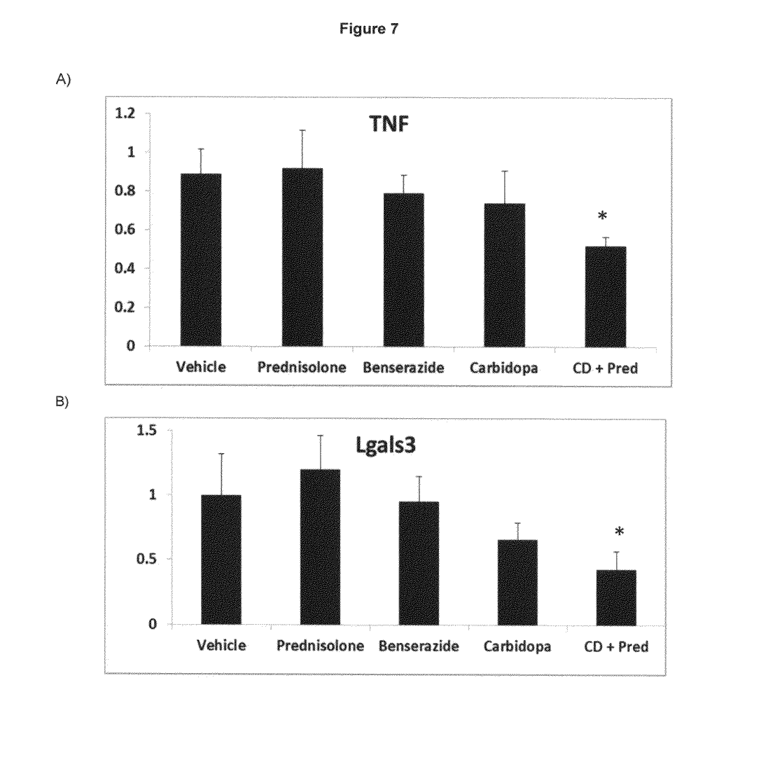

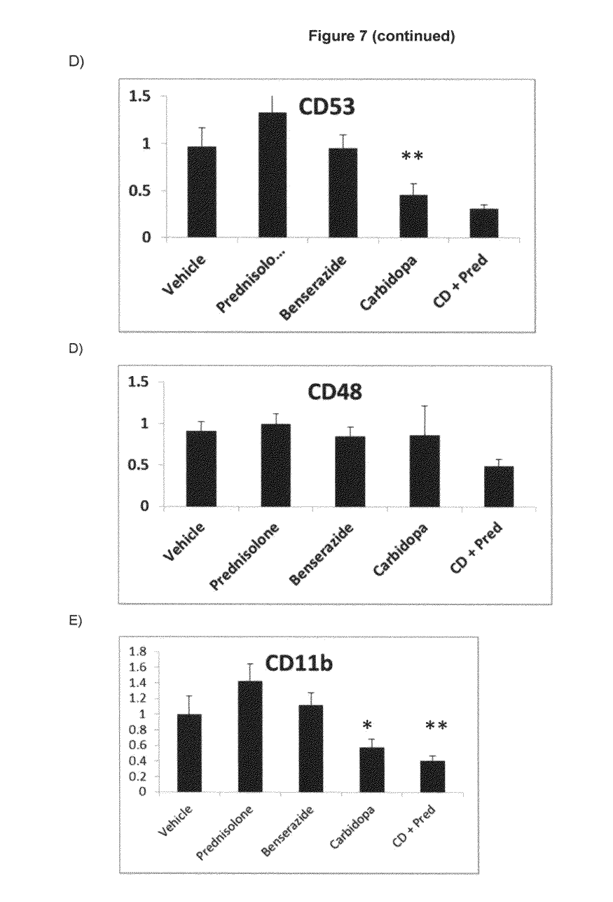

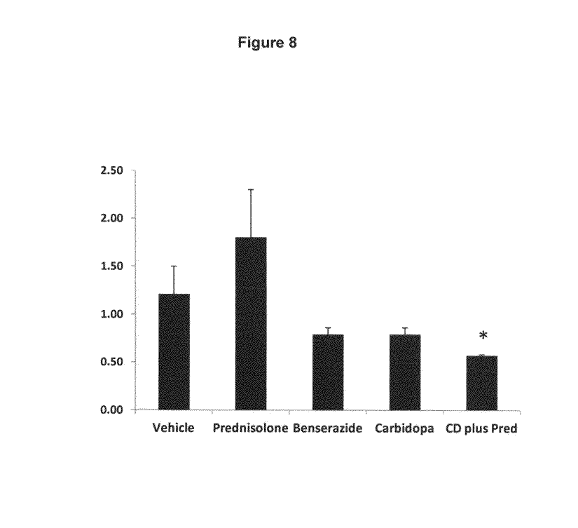

The measured mRNA levels of inflammatory markers in muscles of mdx mice treated with prednisolone, benserazide, carbidopa, or a combination of carbidopa and prednisolone, are shown in the Table below, and in FIG. 7. The inflammatory markers are: (A) TNF; (B) Lgals3; (C) CD53; (D) CD48; (E) CD11b.

| | | Inflammatory marker | | | | |
|---|---|---|---|---|---|---|
| Drug | Dose | TNF | Lgals3 | CD53 | CD48 | CD11b |
| Vehicle | — | 0.89 | 1 | 0.97 | 0.91 | 1 |
| Prednisolone | 0.2 mg/kg ip | 0.92 | 1.2 | 1.33 | 1 | 1.42 |
| | once/day | | | | | |
| Benserazide | 20 mg/kg po | 0.79 | 0.95 | 0.95 | 0.85 | 1.12 |
| | twice/day | | | | | |
| Carbidopa | 50 mg/kg po | 0.74 | 0.66 | 0.46** | 0.86 | 0.58* |
| | twice/day | | | | | |
| Carbidopa + | 50 mg/kg po | 0.52* | 0.43* | 0.31 | 0.49 | 0.41** |
| prednisolone | twice/day | | | | | |
| | 0.2 mg/kg ip | | | | | |
| | once/day | | | | | |

*P < 0.05,
**P < 0.005 compared to vehicle

No significant effect on the level of TNF mRNA in mdx mouse muscle was seen following treatment with prednisolone, benserazide, or carbidopa alone. However, combined treatment with carbidopa and prednisolone reduced the level of TNF mRNA by over 40% (41.6%).

Similarly, no significant effect on the level of Lgals3 or CD48 mRNA levels in mdx mouse muscle was seen following treatment with prednisolone, benserazide, or carbidopa alone. However, combined treatment with carbidopa and prednisolone reduced the level of Lgals3 and CD48 mRNA by 57% and 46.2%, respectively.

The level of CD53 and CD11b mRNA was not significantly affected by treatment with prednisolone or benserazide alone. However, carbidopa alone reduced the level of CD53 mRNA by 52.6%, and the level of CD11b mRNA by 42%. Treatment with a combination of carbidopa and prednisolone was even more effective, reducing the level of CD53 mRNA by 68%, and the level of CD11b mRNA by 59%.

No effects were observed on the mRNA levels of the other inflammatory markers tested (IL-6, Mpeg1, Ly6C1) following treatment with prednisolone, benserazide, or carbidopa.

It was concluded from the results that treatment with neither prednisolone nor benserazide alone was observed to have any statistically significant effect on mRNA levels of the inflammatory markers TNF, Lgals3, CD53, CD48, or CD11b in muscle of mdx mice. However, treatment with carbidopa caused a significant reduction in mRNA level of the B/T cell marker CD53, and the myeloid cell marker CD11b. A synergistic reduction in the mRNA level of all of the inflammatory markers tested was observed following treatment with carbidopa and a glucocorticoid.

It appears from these results that expression of the B/T cell marker CD53, and the myeloid cell marker CD11b, was reduced in mdx mouse muscle by treatment with carbidopa, and that expression of all the inflammatory markers tested was synergistically reduced in mouse mdx muscle by treatment with carbidopa and a glucocorticoid. This suggests that carbidopa, and especially carbidopa and the glucocorticoid in combination, reduced inflammatory cell activation in mdx mouse muscle. This provides evidence that treatment of patients with carbidopa, or with carbidopa and a glucocorticoid in combination, provides an effective treatment to lessen one or more of the symptoms of muscular dystrophy, in particular DMD.

### Effect of Prednisolone, Benserazide, Carbidopa, and a Combination of Carbidopa and Prednisolone, on the mRNA Level of the Pro-Fibrotic Growth Factor TGFβ in Muscles of mdx Mice

The measured mRNA levels of TGFβ in muscles of mdx mice treated with prednisolone, benserazide, carbidopa, or a combination of carbidopa and prednisolone, are shown in the Table below, and in FIG. 8.

| | | | % |
|---|---|---|---|
| | | | change compared |
| Drug | Dose | TGFβ mRNA | to vehicle |
| Vehicle | — | 1.21 | — |
| Prednisolone | 0.2 mg/kg ip once/day | 1.80 | 48.8 |
| Benserazide | 20 mg/kg po twice/day | 0.79 | −34.7 |
| Carbidopa | 50 mg/kg po twice/day | 0.79 | −34.7 |
| Carbidopa + | 50 mg/kg po twice/day | 0.57* | −52.9 |
| prednisolone | 0.2 mg/kg ip once/day | | |

*P < 0.05 compared to vehicle

The results show that treatment with prednisolone alone actually increased the level of TGFβ mRNA in muscle of mdx mice, whereas treatment with benserazide or carbidopa alone each reduced the TGFβ mRNA level by over a third. A synergistic reduction in the TGFβ mRNA level by over 50% was seen with treatment with carbidopa and prednisolone in combination.

It was concluded from these results that treatment with benserazide or carbidopa alone reduced the mRNA level of TGFβ in muscle of mdx mice, and that the effect of carbidopa was synergistically increased following treatment with carbidopa and a glucocorticoid.

It appears from these results that expression of the pro-fibrotic growth factor TGFβ was reduced in mdx mouse muscle by treatment with a VAP-1 inhibitor (carbidopa or benserazide), and that a synergistic reduction in expression of TGF occurred following treatment with a VAP-1 inhibitor and a glucocorticoid in combination. This suggests that a VAP-1 inhibitor (in particular, carbidopa or benserazide), and especially a VAP-1 inhibitor (carbidopa) and a glucocorticoid in combination, had an anti-fibrotic effect in mdx mouse muscle. This provides evidence that treatment of patients with carbidopa, or with carbidopa and a glucocorticoid in combination, provides an effective treatment to lessen one or more of the symptoms of muscular dystrophy, in particular DMD.

General Conclusions Regarding Treatment of Dystrophic Muscle with Benserazide, Carbidopa, or a Combination of Carbidopa and a Glucocorticoid

The results in this example show that:
treatment with a VAP-1 inhibitor, in particular benserazide or carbidopa, reduced infiltration of dystrophic muscle by inflammatory myeloid cells, T helper cells, and DN T cells;
treatment with a VAP-1 inhibitor, in particular carbidopa, changed the relative level of profibrotic and anti-fibrotic myeloid cells in dystrophic muscle in favour of anti-fibrotic myeloid cells in a dose-dependent manner;
the effects of a VAP-1 inhibitor, in particular carbidopa, on reducing infiltration of dystrophic muscle by T helper cells, and DN T cells, were significantly enhanced by co-administration with a glucocorticoid;
the expression levels of inflammatory markers in dystrophic muscle were reduced by treatment with a VAP-1 inhibitor, in particular carbidopa;
the expression levels of several inflammatory markers in dystrophic muscle were synergistically reduced by treatment with a VAP-1 inhibitor, in particular carbidopa, and a glucocorticoid in combination;
the expression level of a pro-fibrotic growth factor in dystrophic muscle was reduced by treatment with a VAP-1 inhibitor, in particular carbidopa or benserazide;
the expression level of a pro-fibrotic growth factor in dystrophic muscle was synergistically reduced by treatment with a VAP-1 inhibitor, in particular carbidopa, and a glucocorticoid in combination.

These results provide evidence that VAP-1 inhibitors (in particular benserazide or carbidopa), can be used to prevent, or treat, one or more symptoms of, or adverse effects attributable to, muscular dystrophy, especially DMD. The results also provide evidence that the therapeutic effects of a VAP-1 inhibitor may be enhanced by combined treatment with a steroid (in particular, a glucocorticoid such as prednisolone).

The results provide evidence that VAP-1 inhibitors (such as benserazide or carbidopa, or a pharmaceutically acceptable salt thereof), or combined treatment with VAP-1 inhibitors and steroids (in particular, treatment with carbidopa, or a pharmaceutically acceptable salt thereof, and a glucocorticoid, such as prednisolone, or a pharmaceutically acceptable salt thereof, in combination), can be used to reduce any of the following symptoms or adverse effects in patients with muscular dystrophy, especially DMD:
muscle pathology;
muscle degeneration;
muscle necrosis;
inflammation of muscle;
infiltration of inflammatory cells into muscle, especially inflammatory myeloid cells such as monocytes, macrophages, neutrophils, or eosinophils;
infiltration of lymphoid cells into muscle, especially T helper lymphocytes or double negative T cells;
inflammatory cell activation in muscle;
muscle fibrosis; or
fibrotic cell activation in muscle; or
   to increase or promote muscle regeneration, or muscle functionality.

What is claimed is:

1. A method of treating muscular dystrophy comprising administering to a subject suffering such disease an effective amount of a VAP-1 inhibitor compound.

2. The method of claim 1, wherein the VAP-1 inhibitor is carbidopa or benserazide, or a pharmaceutically acceptable salt thereof.

3. The method of claim 2, wherein the VAP-1 inhibitor is benserazide, or a pharmaceutically acceptable salt thereof.

4. The method of claim 1, wherein the VAP-1 inhibitor is carbidopa, or a pharmaceutically acceptable salt thereof.

5. The method of claim 1, wherein the VAP-1 inhibitor compound is a polypeptide or protein.

6. The method of claim 1, wherein the muscular dystrophy is selected from Duchenne muscular dystrophy, Becker muscular dystrophy, limb girdle muscular dystrophy, congenital muscular dystrophy and distal muscular dystrophy.

7. A pharmaceutical composition comprising: (a) a VAP-1 inhibitor compound; (b) a steroid; and
(c) a pharmaceutically acceptable carrier, excipient, or diluent.

8. The pharmaceutical composition of claim 7, wherein the steroid is a glucocorticoid, or a pharmaceutically acceptable salt thereof.

9. The pharmaceutical composition of claim 7, wherein the steroid is prednisolone, or a pharmaceutically acceptable salt thereof.

10. The pharmaceutical composition of claim 7, wherein the VAP-1 inhibitor is carbidopa or benserazide, or a pharmaceutically acceptable salt thereof.

11. The pharmaceutical composition of claim 10, wherein the VAP-1 inhibitor is benserazide, or a pharmaceutically acceptable salt thereof.

12. The pharmaceutical composition of claim 7, wherein the VAP-1 inhibitor is carbidopa, or a pharmaceutically acceptable salt thereof.

13. The pharmaceutical composition of claim 7, wherein the VAP-1 inhibitor compound is a polypeptide or protein.

14. A method of treating muscular dystrophy comprising administering to a subject suffering such disease a pharmaceutical composition comprising: (a) a VAP-1 inhibitor compound; (b) a steroid; and
(c) a pharmaceutically acceptable carrier, excipient, or diluent.

15. The method of claim 14, wherein the steroid is a glucocorticoid, or a pharmaceutically acceptable salt thereof.

16. The method of claim 14, wherein the steroid is wherein the steroid is prednisolone, or a pharmaceutically acceptable salt thereof.

17. The method of claim 14, wherein the VAP-1 inhibitor is carbidopa or benserazide, or a pharmaceutically acceptable salt thereof.

18. The method of claim 17, wherein the VAP-1 inhibitor is benserazide, or a pharmaceutically acceptable salt thereof.

19. The method of claim 14, wherein the VAP-1 inhibitor compound is a polypeptide or protein.

20. The method of claim 14, wherein the muscular dystrophy is selected from Duchenne muscular dystrophy, Becker muscular dystrophy, limb girdle muscular dystrophy, congenital muscular dystrophy and distal muscular dystrophy.
